(19) United States
(12) Patent Application Publication
(10) Pub. No.: US 2021/0340245 A1
(43) Pub. Date: Nov. 4, 2021

(54) Materials and Methods for Treating Juvenile Idiopathic Arthritis

(71) Applicant: Janssen Biotech, Inc., Horsham, PA (US)

(72) Inventors: Karen Bensley, Malvern, PA (US); Michael Clark, Warminster, PA (US); Jocelyn Leu, Ambler, PA (US); Zhenhua Xu, Berwyn, PA (US)

(21) Appl. No.: 17/240,609

(22) Filed: Apr. 26, 2021

Related U.S. Application Data
(60) Provisional application No. 63/015,889, filed on Apr. 27, 2020.
(60) Provisional application No. 63/015,894, filed on Apr. 27, 2020.
(60) Provisional application No. 63/015,902, filed on Apr. 27, 2020.

(51) Int. Cl. C07K 16/24; A61K 31/519; A61P 19/02

(52) U.S. Cl. CPC C07K 16/241; A61K 9/0019; A61P 19/02; A61K 31/519

(57) ABSTRACT

The present invention relates to compositions and methods utilizing anti-TNF antibodies, e.g., the anti-TNF antibody golimumab having a heavy chain (HC) comprising an amino acid sequence of SEQ ID NO:36 and a light chain (LC) comprising an amino acid sequence of SEQ ID NO:37, for use in the treatment of juvenile idiopathic arthritis (JIA), and in particular for polyarticular juvenile idiopathic arthritis (pJIA).

### CROSS-REFERENCE TO RELATED APPLICATIONS

This application claims priority to U.S. Provisional Patent Application Ser. No.:

63/015,889, filed Apr. 27, 2020, U.S. Provisional Patent Application Ser. No.: 63/015,894, filed Apr. 27, 2020, and U.S. Provisional Patent Application Ser. No.: 63/015,902, filed Apr. 27, 2020, each of which is incorporated by reference herein in its entirety.

### REFERENCE TO SEQUENCE LISTING SUBMITTED ELECTRONICALLY

This application contains a sequence listing, which is submitted electronically via EFS-Web as an ASCII formatted sequence listing with a file name, JBI6307USNP1SeqListing.txt, creation date of Apr. 20, 2021 and having a size of 25 kb. The sequence listing submitted via EFS-Web is part of the specification and is herein incorporated by reference in its entirety.

### FIELD OF THE INVENTION

The present invention relates to compositions and methods utilizing anti-TNF antibodies, e.g., the anti-TNF antibody golimumab having a heavy chain (HC) comprising an amino acid sequence of SEQ ID NO:36 and a light chain (LC) comprising an amino acid sequence of SEQ ID NO:37, for use in treatment of juvenile idiopathic arthritis (JIA), and in particular for polyarticular juvenile idiopathic arthritis (pJIA).

### BACKGROUND OF THE INVENTION

TNF alpha is a soluble homotrimer of 17 kD protein subunits. A membrane-bound 26 kD precursor form of TNF also exists.

Cells other than monocytes or macrophages also produce TNF alpha. For example, human non-monocytic tumor cell lines produce TNF alpha and CD4+ and CD8+ peripheral blood T lymphocytes and some cultured T and B cell lines also produce TNF alpha.

TNF alpha causes pro-inflammatory actions which result in tissue injury, such as degradation of cartilage and bone, induction of adhesion molecules, inducing procoagulant activity on vascular endothelial cells, increasing the adherence of neutrophils and lymphocytes, and stimulating the release of platelet activating factor from macrophages, neutrophils and vascular endothelial cells.

TNF alpha has been associated with infections, immune disorders, neoplastic pathologies, autoimmune pathologies and graft-versus-host pathologies. The association of TNF alpha with cancer and infectious pathologies is often related to the host's catabolic state. Cancer patients suffer from weight loss, usually associated with anorexia.

The extensive wasting which is associated with cancer, and other diseases, is known as “cachexia”. Cachexia includes progressive weight loss, anorexia, and persistent erosion of lean body mass in response to a malignant growth. The cachectic state causes much cancer morbidity and mortality. There is evidence that TNF alpha is involved in cachexia in cancer, infectious pathology, and other catabolic states.

TNF alpha is believed to play a central role in gram-negative sepsis and endotoxic shock, including fever, malaise, anorexia, and cachexia. Endotoxin strongly activates monocyte/macrophage production and secretion of TNF alpha and other cytokines. TNF alpha and other monocyte-derived cytokines mediate the metabolic and neurohormonal responses to endotoxin. Endotoxin administration to human volunteers produces acute illness with flu-like symptoms including fever, tachycardia, increased metabolic rate and stress hormone release. Circulating TNF alpha increases in patients suffering from Gram-negative sepsis.

Thus, TNF alpha has been implicated in inflammatory diseases, autoimmune diseases, viral, bacterial and parasitic infections, malignancies, and/or neurodegenerative diseases and is a useful target for specific biological therapy in diseases, such as rheumatoid arthritis and Crohn's disease. Beneficial effects in open-label trials with monoclonal antibodies to TNF alpha have been reported with suppression of inflammation and with successful retreatment after relapse in rheumatoid arthritis and in Crohn's disease. Beneficial results in a randomized, double-blind, placebo-controlled trials have also been reported in rheumatoid arthritis with suppression of inflammation.

Neutralizing antisera or mAbs to TNF have been shown in mammals other than man to abrogate adverse physiological changes and prevent death after lethal challenge in experimental endotoxemia and bacteremia. This effect has been demonstrated, e.g., in rodent lethality assays and in primate pathology model systems.

Putative receptor binding loci of hTNF has been disclosed and the receptor binding loci of TNF alpha as consisting of amino acids 11-13, 37-42, 49-57 and 155-157 of TNF have been disclosed.

Non-human mammalian, chimeric, polyclonal (e.g., anti-sera) and/or monoclonal antibodies (Mabs) and fragments (e.g., proteolytic digestion or fusion protein products thereof) are potential therapeutic agents that are being investigated in some cases to attempt to treat certain diseases. However, such antibodies or fragments can elicit an immune response when administered to humans. Such an immune response can result in an immune complex-mediated clearance of the antibodies or fragments from the circulation, and make repeated administration unsuitable for therapy, thereby reducing the therapeutic benefit to the patient and limiting the re-administration of the antibody or fragment. For example, repeated administration of antibodies or fragments comprising non-human portions can lead to serum sickness and/or anaphylaxis. In order to avoid these and other problems, a number of approaches have been taken to reduce the immunogenicity of such antibodies and portions thereof, including chimerization and humanization, as well known in the art. These and other approaches, however, still can result in antibodies or fragments having some immunogenicity, low affinity, low avidity, or with problems in cell culture, scale up, production, and/or low yields. Thus, such antibodies or fragments can be less than ideally suited for manufacture or use as therapeutic proteins.

A need to provide TNF inhibitors that overcame one more of these problems led to development of currently marketed anti-TNF antibodies and other TNF inhibitors, e.g., anti-TNF antibodies such as REMICADE® (infliximab), HUMIRA® (adalimumab), and SIMPONI® (golimumab). Other TNF inhibitors include, e.g., CIMZIA® (certolizumab pegol), a PEGylated antibody fragment, and ENBREL® (etanercept), a soluble TNF receptor fusion protein. For a review of TNF inhibitors, see, e.g., Lis et al., Arch Med Sci. 2014 Dec. 22; 10(6): 1175-1185.

### SUMMARY OF THE INVENTION

The general and preferred embodiments are defined, respectively, by the independent and dependent claims appended hereto, which for the sake of brevity are incorporated by reference herein. Other preferred embodiments, features, and advantages of the various aspects of the invention will become apparent from the detailed description below taken in conjunction with the appended drawing figures.

In certain embodiments, the present invention provides a method of treating juvenile idiopathic arthritis (JIA) in pediatric patients, the method comprising administering an intravenous (IV) dose of an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) inactive disease after 52 weeks of treatment.

In certain embodiments, the present invention provides a method of treating juvenile idiopathic arthritis (JIA) in pediatric patients, the method comprising administering an intravenous (IV) dose of an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein >30% of the pediatric patients meet the criteria for JIA ACR inactive disease after 52 weeks of treatment.

In certain embodiments, the present invention provides a method of treating juvenile idiopathic arthritis (JIA) in pediatric patients, the method comprising administering an intravenous (IV) dose of an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) inactive disease after 52 weeks of treatment and said pediatric patients are 2 to <18 years old.

In certain embodiments, the present invention provides a method of treating juvenile idiopathic arthritis (JIA) in pediatric patients, the method comprising administering an intravenous (IV) dose of an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) inactive disease after 52 weeks of treatment and said juvenile idiopathic arthritis (JIA) is polyarticular juvenile idiopathic arthritis (pJIA).

In certain embodiments, the present invention provides a method of treating juvenile idiopathic arthritis (JIA) in pediatric patients, the method comprising administering an intravenous (IV) dose of an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) inactive disease after 52 weeks of treatment and the IV dose is 80 mg/m² of the anti-TNF antibody, at weeks 0, 4, and then every 8 weeks thereafter.

In certain embodiments, the present invention provides a method of treating juvenile idiopathic arthritis (JIA) in pediatric patients, the method comprising administering an intravenous (IV) dose of an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) inactive disease after 52 weeks of treatment and the method further comprises administering methotrexate (MTX) to the pediatric patients.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) inactive disease after 52 weeks of treatment.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein >30% of the pediatric patients meet the criteria for JIA ACR inactive disease after 52 weeks of treatment.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) inactive disease after 52 weeks of treatment and said pediatric patients are 2 to <18 years old.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) inactive disease after 52 weeks of treatment and said juvenile idiopathic arthritis (JIA) is polyarticular juvenile idiopathic arthritis (pJIA).

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) inactive disease after 52 weeks of treatment and the IV dose of the composition comprises 80mg/m² of the anti-TNF antibody, at weeks 0, 4, and then every 8 weeks thereafter.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) inactive disease after 52 weeks of treatment and the method further comprises administering methotrexate (MTX) to the pediatric patients.

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) clinical remission after 52 weeks of treatment.

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein >10% of the pediatric patients meet the criteria for JIA ACR clinical remission after 52 weeks of treatment.

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) clinical remission after 52 weeks of treatment and said pediatric patients are 2 to <18 years old.

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) clinical remission after 52 weeks of treatment and said juvenile idiopathic arthritis (JIA) is polyarticular juvenile idiopathic arthritis (pJIA).

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) clinical remission after 52 weeks of treatment and the IV dose is 80 mg/m² of the anti-TNF antibody, at weeks 0, 4, and then every 8 weeks thereafter.

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) clinical remission after 52 weeks of treatment and the method further comprises administering methotrexate (MTX) to the pediatric patients.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) clinical remission after 52 weeks of treatment.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein >30% of the pediatric patients meet the criteria for JIA ACR inactive disease after 52 weeks of treatment.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) clinical remission after 52 weeks of treatment and said pediatric patients are 2 to <18 years old.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) clinical remission after 52 weeks of treatment and said juvenile idiopathic arthritis (JIA) is polyarticular juvenile idiopathic arthritis (pJIA).

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) clinical remission after 52 weeks of treatment and the IV dose of the composition comprises 80mg/m² of the anti-TNF antibody, at weeks 0, 4, and then every 8 weeks thereafter.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein the pediatric patients meet the criteria for JIA American College of Rheumatology (JIA ACR) clinical remission after 52 weeks of treatment and the method further comprises administering methotrexate (MTX) to the pediatric patients.

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein >50% of the pediatric patients meet the criteria for JIA American College of Rheumatology (JIA ACR) 30, JIA ACR 50, and JIA ACR 70 after 52 weeks of treatment.

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein >50% of the pediatric patients meet the criteria for JIA American College of Rheumatology (JIA ACR) 30, JIA ACR 50, and JIA ACR 70 after 52 weeks of treatment and said pediatric patients are 2 to <18 years old.

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein >50% of the pediatric patients meet the criteria for JIA American College of Rheumatology (JIA ACR) 30, JIA ACR 50, and JIA ACR 70 after 52 weeks of treatment and said juvenile idiopathic arthritis (JIA) is polyarticular juvenile idiopathic arthritis (pJIA).

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein >50% of the pediatric patients meet the criteria for JIA American College of Rheumatology (JIA ACR) 30, JIA ACR 50, and JIA ACR 70 after 52 weeks of treatment and the IV dose is 80 mg/m² of the anti-TNF antibody, at weeks 0, 4, and then every 8 weeks thereafter.

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein >50% of the pediatric patients meet the criteria for JIA American College of Rheumatology (JIA ACR) 30, JIA ACR 50, and JIA ACR 70 after 52 weeks of treatment and the method further comprises administering methotrexate (MTX) to the pediatric patients.

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein >20% of the pediatric patients have a Juvenile Arthritis Disease Activity Score counting 71 joints (JADAS 71) for low disease activity after 52 weeks of treatment.

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein >20% of the pediatric patients have a Juvenile Arthritis Disease Activity Score counting 71 joints (JADAS 71) for low disease activity after 52 weeks of treatment and said pediatric patients are 2 to <18 years old.

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein >20% of the pediatric patients have a Juvenile Arthritis Disease Activity Score counting 71 joints (JADAS 71) for low disease activity after 52 weeks of treatment and said juvenile idiopathic arthritis (JIA) is polyarticular juvenile idiopathic arthritis (pJIA).

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein >20% of the pediatric patients have a Juvenile Arthritis Disease Activity Score counting 71 joints (JADAS 71) for low disease activity after 52 weeks of treatment and the IV dose is 80 mg/m² of the anti-TNF antibody, at weeks 0, 4, and then every 8 weeks thereafter.

In certain embodiments, the present invention provides a method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein >20% of the pediatric patients have a Juvenile Arthritis

Disease Activity Score counting 71 joints (JADAS 71) for low disease activity after 52 weeks of treatment and the method further comprises administering methotrexate (MTX) to the pediatric patients.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein >50% of the pediatric patients meet the criteria for JIA American College of Rheumatology (JIA ACR) 30, JIA ACR 50, and JIA ACR 70 after 52 weeks of treatment.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein >50% of the pediatric patients meet the criteria for JIA American College of Rheumatology (JIA ACR) 30, JIA ACR 50, and JIA ACR 70 after 52 weeks of treatment and said pediatric patients are 2 to <18 years old.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein >50% of the pediatric patients meet the criteria for JIA American College of Rheumatology (JIA ACR) 30, JIA ACR 50, and JIA ACR 70 after 52 weeks of treatment and said juvenile idiopathic arthritis (JIA) is polyarticular juvenile idiopathic arthritis (pJIA).

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein >50% of the pediatric patients meet the criteria for JIA American College of Rheumatology (JIA ACR) 30, JIA ACR 50, and JIA ACR 70 after 52 weeks of treatment and the IV dose of the composition comprises 80 mg/m² of the anti-TNF antibody, at weeks 0, 4, and then every 8 weeks thereafter.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein >50% of the pediatric patients meet the criteria for JIA American College of Rheumatology (JIA ACR) 30, JIA ACR 50, and JIA ACR 70 after 52 weeks of treatment and the method further comprises administering methotrexate (MTX) to the pediatric patients.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein >20% of the pediatric patients have a Juvenile Arthritis Disease Activity Score counting 71 joints (JADAS 71) for low disease activity after 52 weeks of treatment.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein >20% of the pediatric patients have a Juvenile Arthritis Disease Activity Score counting 71 joints (JADAS 71) for low disease activity after 52 weeks of treatment and said pediatric patients are 2 to <18 years old.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein >20% of the pediatric patients have a Juvenile Arthritis Disease Activity Score counting 71 joints (JADAS 71) for low disease activity after 52 weeks of treatment and said juvenile idiopathic arthritis (JIA) is polyarticular juvenile idiopathic arthritis (pJIA).

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein >20% of the pediatric patients have a Juvenile Arthritis Disease Activity Score counting 71 joints (JADAS 71) for low disease activity after 52 weeks of treatment and the IV dose of the composition comprises 80 mg/m² of the anti-TNF antibody, at weeks 0, 4, and then every 8 weeks thereafter.

In certain embodiments, the present invention provides a method for treating juvenile idiopathic arthritis (JIA) in pediatric patients, the method comprising administering an intravenous (IV) dose of a composition comprising an anti-TNF antibody to the pediatric patients, wherein the anti-TNF antibody comprises a heavy chain (HC) comprising an amino acid sequence of SEQ ID NO:36 and a light chain (LC) comprising an amino acid sequence of SEQ ID NO:37, and wherein >20% of the pediatric patients have a Juvenile Arthritis Disease Activity Score counting 71 joints (JADAS 71) for low disease activity after 52 weeks of treatment and the method further comprises administering methotrexate (MTX) to the pediatric patients.

### DESCRIPTION OF THE FIGURES

FIG. 1 shows a graphical representation showing an assay for ability of TNV mAbs in hybridoma cell supernatants to inhibit TNFα binding to recombinant TNF receptor. Varying amounts of hybridoma cell supernatants containing known amounts of TNV mAb were preincubated with a fixed concentration (5 ng/ml) of ¹²⁵I-labeled TNFα. The mixture was transferred to 96-well Optiplates that had been previously coated with p55-sf2, a recombinant TNF receptor/IgG fusion protein. The amount of TNFα that bound to the p55 receptor in the presence of the mAbs was determined after washing away the unbound material and counting using a gamma counter. Although eight TNV mAb samples were tested in these experiments, for simplicity three of the mAbs that were shown by DNA sequence analyses to be identical to one of the other TNV mAbs are not shown here. Each sample was tested in duplicate. The results shown are representative of two independent experiments.

FIG. 2A-B shows DNA sequences of the TNV mAb heavy chain variable regions. The germline gene shown is the DP-46 gene. ‘TNVs’ indicates that the sequence shown is the sequence of TNV14, TNV15, TNV148, and TNV196. The first three nucleotides in the TNV sequence define the translation initiation Met codon. Dots in the TNV mAb gene sequences indicate the nucleotide is the same as in the germline sequence. The first 19 nucleotides (underlined) of the TNV sequences correspond to the oligonucleotide used to PCR-amplify the variable region. An amino acid translation (single letter abbreviations) starting with the mature mAb is shown only for the germline gene. The three CDR domains in the germline amino acid translation are marked in bold and underlined. Lines labeled TNV148(B) indicate that the sequence shown pertains to both TNV148 and TNV148B. Gaps in the germline DNA sequence (CDR3) were due to the sequence not being known or not existing in the germline gene at the time. The TNV mAb heavy chains use the J6 joining region.

FIG. 3 shows DNA sequences of the TNV mAb light chain variable regions. The germline gene shown is a representative member of the Vg/38K family of human kappa germline variable region genes. Dots in the TNV mAb gene sequences indicate the nucleotide is the same as in the germline sequence. The first 16 nucleotides (underlined) of the TNV sequences correspond to the oligonucleotide used to PCR-amplify the variable region. An amino acid translation of the mature mAb (single letter abbreviations) is shown only for the germline gene. The three CDR domains in the germline amino acid translation are marked in bold and underlined. Lines labeled TNV148(B) indicate that the sequence shown pertains to both TNV148 and TNV148B. Gaps in the germline DNA sequence (CDR3) are due to the sequence not being known or not existing in the germline gene. The TNV mAb light chains use the J3 joining sequence.

FIG. 4 shows deduced amino acid sequences of the TNV mAb heavy chain variable regions. The amino acid sequences shown (single letter abbreviations) were deduced from DNA sequence determined from both uncloned PCR products and cloned PCR products. The amino sequences are shown partitioned into the secretory signal sequence (signal), framework (FW), and complementarity determining region (CDR) domains. The amino acid sequence for the DP-46 germline gene is shown on the top line for each domain. Dots indicate that the amino acid in the TNV mAb is identical to the germline gene. TNV148(B) indicates that the sequence shown pertains to both TNV148 and TNV148B. ‘TNVs’ indicates that the sequence shown pertains to all TNV mAbs unless a different sequence is shown. Dashes in the germline sequence (CDR3) indicate that the sequences are not known or do not exist in the germline gene.

FIG. 5 shows deduced amino acid sequences of the TNV mAb light chain variable regions. The amino acid sequences shown (single letter abbreviations) were deduced from DNA sequence determined from both uncloned PCR products and cloned PCR products. The amino sequences are shown partitioned into the secretory signal sequence (signal), framework (FW), and complementarity determining region (CDR) domains. The amino acid sequence for the Vg/38K-type light chain germline gene is shown on the top line for each domain. Dots indicate that the amino acid in the TNV mAb is identical to the germline gene. TNV148 (B) indicates that the sequence shown pertains to both TNV148 and TNV148B. ‘All’ indicates that the sequence shown pertains to TNV14, TNV15, TNV148, TNV148B, and TNV186.

FIG. 6 shows schematic illustrations of the heavy and light chain expression plasmids used to make the rTNV148B-expressing C466 cells. p1783 is the heavy chain plasmid and p1776 is the light chain plasmid. The rTNV148B variable and constant region coding domains are shown as black boxes. The immunoglobulin enhancers in the J-C introns are shown as gray boxes. Relevant restriction sites are shown. The plasmids are shown oriented such that transcription of the Ab genes proceeds in a clockwise direction. Plasmid p1783 is 19.53 kb in length and plasmid p1776 is 15.06 kb in length. The complete nucleotide sequences of both plasmids are known. The variable region coding sequence in p1783 can be easily replaced with another heavy chain variable region sequence by replacing the BsiWI/BstBI restriction fragment. The variable region coding sequence in p1776 can be replaced with another variable region sequence by replacing the SalI/AfIII restriction fragment.

FIG. 7 shows graphical representation of growth curve analyses of five rTNV148B-producing cell lines. Cultures were initiated on day 0 by seeding cells into T75 flasks in I5Q+MHX media to have a viable cell density of 1.0×10⁵ cells/ml in a 30 ml volume. The cell cultures used for these studies had been in continuous culture since transfections and subclonings were performed. On subsequent days, cells in the T flasks were thoroughly resuspended and a 0.3 ml aliquot of the culture was removed. The growth curve studies were terminated when cell counts dropped below 1.5×10⁵ cells/ml. The number of live cells in the aliquot was determined by trypan blue exclusion and the remainder of the aliquot stored for later mAb concentration determination. An ELISA for human IgG was performed on all sample aliquots at the same time.

FIG. 8 shows a graphical representation of the comparison of cell growth rates in the presence of varying concentrations of MHX selection. Cell subclones C466A and C466B were thawed into MHX-free media (IMDM, 5% FBS, 2 mM glutamine) and cultured for two additional days. Both cell cultures were then divided into three cultures that contained either no MHX, 0.2×MHX, or 1×MHX. One day later, fresh T75 flasks were seeded with the cultures at a starting density of 1×10⁵ cells/ml and cells counted at 24 hour intervals for one week. Doubling times during the first 5 days were calculated using the formula in SOP PD32.025 and are shown above the bars.

FIG. 9 shows graphical representations of the stability of mAb production over time from two rTNV148B-producing cell lines. Cell subclones that had been in continuous culture since performing transfections and subclonings were used to start long-term serial cultures in 24-well culture dishes. Cells were cultured in I5Q media with and without MHX selection. Cells were continually passaged by splitting the cultures every 4 to 6 days to maintain new viable cultures while previous cultures were allowed to go spent. Aliquots of spent cell supernatant were collected shortly after cultures were spent and stored until the mAb concentrations were determined. An ELISA for human IgG was performed on all sample aliquots at the same time.

FIG. 10 shows arthritis mouse model mice Tg 197 weight changes in response to anti-TNF antibodies of the present invention as compared to controls in Example 4. At approximately 4 weeks of age the Tg197 study mice were assigned, based on gender and body weight, to one of 9 treatment groups and treated with a single intraperitoneal bolus dose of Dulbecco's PBS (D-PBS) or an anti-TNF antibody of the present invention (TNV14, TNV148 or TNV196) at either 1 mg/kg or 10 mg/kg. When the weights were analyzed as a change from pre-dose, the animals treated with 10 mg/kg cA2 showed consistently higher weight gain than the D-PBS-treated animals throughout the study. This weight gain was significant at weeks 3-7. The animals treated with 10 mg/kg TNV148 also achieved significant weight gain at week 7 of the study.

FIG. 11A-C represent the progression of disease severity based on the arthritic index as presented in Example 4. The 10 mg/kg cA2-treated group's arthritic index was lower than the D-PBS control group starting at week 3 and continuing throughout the remainder of the study (week 7). The animals treated with 1 mg/kg TNV14 and the animals treated with 1 mg/kg cA2 failed to show significant reduction in AI after week 3 when compared to the D-PBS-treated Group. There were no significant differences between the 10 mg/kg treatment groups when each was compared to the others of similar dose (10 mg/kg cA2 compared to 10 mg/kg TNV14, 148 and 196). When the 1 mg/kg treatment groups were compared, the 1 mg/kg TNV148 showed a significantly lower AI than 1 mg/kg cA2 at 3, 4 and 7 weeks. The 1 mg/kg TNV148 was also significantly lower than the 1 mg/kg TNV14-treated Group at 3 and 4 weeks. Although TNV196 showed significant reduction in AI up to week 6 of the study (when compared to the D-PBS-treated Group), TNV148 was the only 1 mg/kg treatment that remained significant at the conclusion of the study.

FIG. 12 shows arthritis mouse model mice Tg 197 weight changes in response to anti-TNF antibodies of the present invention as compared to controls in Example 5. At approximately 4 weeks of age the Tg197 study mice were assigned, based on body weight, to one of 8 treatment groups and treated with a intraperitoneal bolus dose of control article (D-PBS) or antibody (TNV14, TNV148) at 3 mg/kg (week 0). Injections were repeated in all animals at weeks 1, 2, 3, and 4. Groups 1-6 were evaluated for test article efficacy. Serum samples, obtained from animals in Groups 7 and 8 were evaluated for immune response inductively and pharmacokinetic clearance of TNV14 or TNV148 at weeks 2, 3 and 4.

FIG. 13A-C are graphs representing the progression of disease severity in Example 5 based on the arthritic index. The 10 mg/kg cA2-treated group's arthritic index was significantly lower than the D-PBS control group starting at week 2 and continuing throughout the remainder of the study (week 5). The animals treated with 1 mg/kg or 3 mg/kg of cA2 and the animals treated with 3 mg/kg TNV14 failed to achieve any significant reduction in AI at any time throughout the study when compared to the d-PBS control group. The animals treated with 3 mg/kg TNV148 showed a significant reduction when compared to the d-PBS-treated group starting at week 3 and continuing through week 5. The 10 mg/kg cA2-treated animals showed a significant reduction in AI when compared to both the lower doses (1 mg/kg and 3 mg/kg) of cA2 at weeks 4 and 5 of the study and was also significantly lower than the TNV14-treated animals at weeks 3-5. Although there appeared to be no significant differences between any of the 3 mg/kg treatment groups, the AI for the animals treated with 3 mg/kg TNV14 were significantly higher at some time points than the 10 mg/kg whereas the animals treated with TNV148 were not significantly different from the animals treated with 10 mg/kg of cA2.

FIG. 14 shows arthritis mouse model mice Tg 197 weight changes in response to anti-TNF antibodies of the present invention as compared to controls in Example 6. At approximately 4 weeks of age the Tg197 study mice were assigned, based on gender and body weight, to one of 6 treatment groups and treated with a single intraperitoneal bolus dose of antibody (cA2, or TNV148) at either 3 mg/kg or 5 mg/kg. This study utilized the D-PBS and 10 mg/kg cA2 control Groups.

FIG. 15 represents the progression of disease severity based on the arthritic index as presented in Example 6. All treatment groups showed some protection at the earlier time points, with the 5 mg/kg cA2 and the 5 mg/kg TNV148 showing significant reductions in AI at weeks 1-3 and all treatment groups showing a significant reduction at week 2. Later in the study the animals treated with 5 mg/kg cA2 showed some protection, with significant reductions at weeks 4, 6 and 7. The low dose (3 mg/kg) of both the cA2 and the TNV148 showed significant reductions at 6 and all treatment groups showed significant reductions at week 7. None of the treatment groups were able to maintain a significant reduction at the conclusion of the study (week 8). There were no significant differences between any of the treatment groups (excluding the saline control group) at any time point.

FIG. 16 shows arthritis mouse model mice Tg 197 weight changes in response to anti-TNF antibodies of the present invention as compared to controls in Example 7. To compare the efficacy of a single intraperitoneal dose of TNV148 (derived from hybridoma cells) and rTNV148B (derived from transfected cells). At approximately 4 weeks of age the Tg197 study mice were assigned, based on gender and body weight, to one of 9 treatment groups and treated with a single intraperitoneal bolus dose of Dulbecco's PBS (D-PBS) or antibody (TNV148, rTNV148B) at 1 mg/kg.

FIG. 17 represents the progression of disease severity based on the arthritic index as presented in Example 7. The 10 mg/kg cA2-treated group's arthritic index was lower than the D-PBS control group starting at week 4 and continuing throughout the remainder of the study (week 8). Both of the TNV148-treated Groups and the 1 mg/kg cA2-treated Group showed a significant reduction in AI at week 4. Although a previous study (P-099-017) showed that TNV148 was slightly more effective at reducing the Arthritic Index following a single 1 mg/kg intraperitoneal bolus, this study showed that the AI from both versions of the TNV antibody-treated groups was slightly higher. Although (with the exception of week 6) the 1 mg/kg cA2-treated Group was not significantly increased when compared to the 10 mg/kg cA2 group and the TNV148-treated Groups were significantly higher at weeks 7 and 8, there were no significant differences in AI between the 1 mg/kg cA2, 1 mg/kg TNV148 and 1 mg/kg TNV148B at any point in the study.

FIG. 18 shows a diagram of the pJIA clinical study design. DBL=Database Lock, LTE=Long-term extension, MSE=Major secondary endpoint, PE=Primary endpoint. Golimumab 80 mg/m² IV infusions are marked with an arrow at the indicated times. Patients also received commercial MTX through at least Week 28 at the same weekly BSA-based dose as at the time of study entry.

FIG. 19 shows a diagram for patient disposition throughout the study. *Adds up to 51 because 1 patient had more than 1 reason for ineligibility. AE, adverse event; JIA, juvenile idiopathic arthritis; n, number of patients.

FIG. 20A-B show observed steady-state serum trough golimumab concentrations (μg/mL) (A) and model-predicted AUC_{SS} of serum golimumab concentration (μg·day/mL) (B) at Week 28 by age group in patients with poly-JIA and in the adult RA reference population. The horizontal lines within the boxes represent the medians, the lower edges of the boxes represent the 1st quartile, and the upper edges of the boxes represent the 3rd quartile. Whiskers represent the most extreme observations within the 1.5× interquartile range. AUC_{SS}, steady-state area under the curve; JIA, juvenile idiopathic arthritis; n, number of patients in the population; RA, rheumatoid arthritis; WK, week.

FIG. 21A-D show clinical efficacy through Week 52; percentage of JIA ACR 30/50/70/90 responders (A) percentage of patients with JIA ACR inactive disease or clinical remission on medication (B), mean (standard deviation) CHAQ and parent assessment of pain scores (C), and mean (95% confidence interval) JADAS 71 scores (D). For (A), N=127 at all the time points for JIA ACR 30, 50, 70, and 90; per the ITT principle, missing data were treated per NRI and LOCF. For (B), N=127 at all the time points for inactive disease and clinical remission on medication. Clinical remission on medication was defined as inactive disease at each visit for a period of ≥6 months while on medication for poly-JIA (all visits encompassing at least 24 weeks prior had to meet the inactive disease criteria). Per the ITT principle, for inactive disease and clinical remission, missing data were treated per LOCF and NRI. For (C), Pain and CHAQ scores were based on observed data. For (D), JADAS score was based on observed data. 95% confidence interval is based on normal approximation: mean±1.96×SD/√N. ACR, American College of Rheumatology; BSL, baseline; CHAQ, Childhood Health Assessment Questionnaire; HDA, high disease activity; ID, inactive disease; ITT, intention-to-treat; JADAS, Juvenile Arthritis Disease Activity Score; JIA, juvenile idiopathic arthritis; LDA, low disease activity; LOCF, last observation carried forward; N, all treated patients; n, number of evaluable patients; NRI, non-responder imputation; SD, standard deviation.

### DESCRIPTION OF THE INVENTION

The present invention provides compositions comprising anti-TNF antibodies having a heavy chain (HC) comprising SEQ ID NO:36 and a light chain (LC) comprising SEQ ID NO:37 and manufacturing processes for producing such anti-TNF antibodies.

As used herein, an “anti-tumor necrosis factor alpha antibody,” “anti-TNF antibody,” “anti-TNF antibody portion,” or “anti-TNF antibody fragment” and/or “anti-TNF antibody variant” and the like include any protein or peptide containing molecule that comprises at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework region, or any portion thereof, or at least one portion of an TNF receptor or binding protein, which can be incorporated into an antibody of the present invention. Such antibody optionally further affects a specific ligand, such as but not limited to where such antibody modulates, decreases, increases, antagonizes, agonizes, mitigates, alleviates, blocks, inhibits, abrogates and/or interferes with at least one TNF activity or binding, or with TNF receptor activity or binding, in vitro, in situ and/or in vivo. As a non-limiting example, a suitable anti-TNF antibody, specified portion or variant of the present invention can bind at least one TNF, or specified portions, variants or domains thereof. A suitable anti-TNF antibody, specified portion, or variant can also optionally affect at least one of TNF activity or function, such as but not limited to, RNA, DNA or protein synthesis, TNF release, TNF receptor signaling, membrane TNF cleavage, TNF activity, TNF production and/or synthesis. The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian TNF. For example, antibody fragments capable of binding to TNF or portions thereof, including, but not limited to Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the CH₁ domain and/or hinge region of the heavy chain. The various portions of antibodies can be joined together chemically by conventional techniques or can be prepared as a contiguous protein using genetic engineering techniques.

As used herein, the term “human antibody” refers to an antibody in which substantially every part of the protein (e.g., CDR, framework, C_{L}, C_{H} domains (e.g., C_{H}1, C_{H}2, and CH3), hinge, (V_{L}, V_{H})) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

Bispecific, e.g., DuoBody® (bispecific antibody), heterospecific, heteroconjugate or similar antibodies can also be used that are monoclonal, preferably human or humanized, antibodies that have binding specificities for at least two different antigens. In the present case, one of the binding specificities is for at least one TNF protein, the other one is for any other antigen. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, can be cumbersome with low product yields and different strategies have been developed to facilitate bispecific antibody production.

Full length bispecific antibodies can be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each can bind a distinct epitope.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Intl. Publ. No. WO 2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. US2010/028637 or US Pat. Publ. No. US2011/0123532. In other strategies, heterodimerization may be promoted by following substitutions (expressed as modified position in the first CH3 domain of the first heavy chain/modified position in the second CH3 domain of the second heavy chain): L351Y_F405A_Y407V/T394W, T366_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S. Pat. Publ. No. US2012/0149876 or U.S. Pat. Publ. No. US2013/0195849.

In addition to methods described above, bispecific antibodies can be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl. Pat. Publ. No. WO2011/131746. In the methods, the first monospecific bivalent antibody and the second monospecific bivalent antibody are engineered to have certain substitutions at the CH3 domain that promoter heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Anti-TNF antibodies (also termed TNF antibodies) useful in the methods and compositions of the present invention can optionally be characterized by high affinity binding to TNF and optionally and preferably having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), entirely incorporated herein by reference).

Utility: The isolated nucleic acids of the present invention can be used for production of at least one anti-TNF antibody or specified variant thereof, which can be used to measure or effect in an cell, tissue, organ or animal (including mammals and humans), to diagnose, monitor, modulate, treat, alleviate, help prevent the incidence of, or reduce the symptoms of, at least one TNF condition, selected from, but not limited to, at least one of an immune disorder or disease, a cardiovascular disorder or disease, an infectious, malignant, and/or neurologic disorder or disease.

Such a method can comprise administering an effective amount of a composition or a pharmaceutical composition comprising at least one anti-TNF antibody to a cell, tissue, organ, animal or patient in need of such modulation, treatment, alleviation, prevention, or reduction in symptoms, effects or mechanisms. The effective amount can comprise an amount of about 0.001 to 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts. Citations. All publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

Antibodies of the Present Invention: At least one anti-TNF antibody of the present invention comprising all of the heavy chain variable CDR regions of SEQ ID NOS:1, 2 and 3 and/or all of the light chain variable CDR regions of SEQ ID NOS:4, 5 and 6 can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001), each entirely incorporated herein by reference.

Human antibodies that are specific for human TNF proteins or fragments thereof can be raised against an appropriate immunogenic antigen, such as isolated and/or TNF protein or a portion thereof (including synthetic molecules, such as synthetic peptides). Other specific or general mammalian antibodies can be similarly raised. Preparation of immunogenic antigens, and monoclonal antibody production can be performed using any suitable technique.

In one approach, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMAIWA, NEURO 2A, or the like, or heteromylomas, fusion products thereof, or any cell or fusion cell derived therefrom, or any other suitable cell line as known in the art. See, e.g., www.atcc.org, www.lifetech.com., and the like, with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, entirely incorporated herein by reference.

Antibody producing cells can also be obtained from the peripheral blood or, preferably the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; BioInvent, Lund, Sweden; Dyax Corp., Enzon, Affymax/Biosite; Xoma, Berkeley, Calif.; Ixsys. See, e.g., EP 368,684, PCT/GB91/01134; PCT/GB92/01755; PCT/GB92/002240; PCT/GB92/00883; PCT/GB93/00605; US 08/350260(May 12, 1994); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); EP 614 989 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins—U.S. Pat. Nos. 5,723,323, 5,763,192, 5,814,476, 5,817,483, 5,824,514, 5,976,862, WO 86/05803, EP 590 689 (Ixsys, now Applied Molecular Evolution (AME), each entirely incorporated herein by reference) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and applications) that are capable of producing a repertoire of human antibodies, as known in the art and/or as described herein. Such techniques include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (November 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, Mass.; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Methods for engineering or humanizing non-human or human antibodies can also be used and are well known in the art. Generally, a humanized or engineered antibody has one or more amino acid residues from a source which is non-human, e.g., but not limited to mouse, rat, rabbit, non-human primate or other mammal. These human amino acid residues are often referred to as “import” residues, which are typically taken from an “import” variable, constant or other domain of a known human sequence.

Known human Ig sequences are disclosed in numerous publications and websites, for example:

www.ncbi.nlm.nih.gov/entrez/query.fcgi;

www.atcc.org/phage/hdb.html;

www.sciquest.com/;

www.abcam.com/;

www.antibodyresource.com/onlinecomp.html;

www.public.iastate.edu/˜pedro/research_tools.html;

www.mgen.uni-heidelberg.de/SD/IT/IT.html;

www.whfreeman.com/immunology/CH05/kuby05.htm;

www.library.thinkquest.org/12429/Immune/Antibody.html;

www.hhmi.org/grants/lectures/1996/vlab/;

www.path.cam.ac.uk/˜mrc7/mikeimages.html;

www.antibodyresource.com/;

www.mcb.harvard.edu/BioLinks/Immunology.html.

www.immunologylink.com/;

www.pathbox.wustl.edu/˜hcenter/index.html;

www.biotech.ufl.edu/˜hcl/;

www.pebio.com/pa/340913/340913.html;

www.nal.usda.gov/awic/pubs/antibody/;

www.m.ehime-u.ac.jp/˜yasuhito/Elisa.html;

www.biodesign.com/table.asp;

www.icnet.uk/axp/facs/davies/links.html;

www.biotech.ufl.edu/˜fccl/protocol.html;

www.isac-net.org/sites_geo.html;

www.aximt1.imt.uni-marburg.de/˜rek/AEPStart.html;

www.baserv.uci.kun.nl/˜jraats/links1.html;

www.recab.uni-hd.de/immuno.bme.nwu.edu/;

www.mrc-cpe.cam.ac.uk/imt-doc/public/INTRO.html;

www.ibt.unam.mx/virN_mice.html; imgt.cnusc.fr:8104/;

www.biochem.ucl.ac.uk/˜martin/abs/index.html; antibody.bath.ac.uk/;

www.abgen.cvm.tamu.edu/lab/

www.abgen.html;

www.unizh.ch/˜honegger/AHOseminar/Slide01.html;

www.cryst.bbk.ac.uk/˜ubcg07s/;

www.nimr.mrc.ac.uk/CC/ccaewg/ccaewg.htm;

www.path.cam.ac.uk/˜mrc7/humanisation/TAHHP.html;

www.ibt.unam.mx/vir/structure/stat_aim.html;

www.biosci.missouri.edu/smithgp/index.html;

www.cryst.bioc.cam.ac.uk/˜fmolina/Web-pages/Pept/spottech.html;

www.jerini.de/frproducts.html;

www.patents.ibm.com/ibm.html.Kabat et al.,

Sequences of Proteins of Immunological Interest, U.S. Dept. Health (1983), each entirely incorporated herein by reference.

Such imported sequences can be used to reduce immunogenicity or reduce, enhance or modify binding, affinity, on-rate, off-rate, avidity, specificity, half-life, or any other suitable characteristic, as known in the art. Generally, part or all of the non-human or human CDR sequences are maintained while the non-human sequences of the variable and constant regions are replaced with human or other amino acids. antibodies can also optionally be humanized with retention of high affinity for the antigen and other favorable biological properties. To achieve this goal, humanized antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Humanization or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos: 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, PCT/: US98/16280, US96/18978, US91/09630, US91/05939, US94/01234, GB89/01334, GB91/01134, GB92/01755; WO90/14443, WO90/14424, WO90/14430, EP 229246, each entirely incorporated herein by reference, included references cited therein.

The anti-TNF antibody can also be optionally generated by immunization of a transgenic animal (e.g., mouse, rat, hamster, non-human primate, and the like) capable of producing a repertoire of human antibodies, as described herein and/or as known in the art. Cells that produce a human anti-TNF antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos: 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Screening antibodies for specific binding to similar proteins or fragments can be conveniently achieved using peptide display libraries. This method involves the screening of large collections of peptides for individual members having the desired function or structure. antibody screening of peptide display libraries is well known in the art. The displayed peptide sequences can be from 3 to 5000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278. Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, Calif.), and Cambridge antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, U.S. Pat. Nos. 5,427,908, 5,580,717, assigned to Affymax; U.S. Pat. No. 5,885,793, assigned to Cambridge antibody Technologies; U.S. Pat. No. 5,750,373, assigned to Genentech, U.S. Pat. Nos. 5,618,920, 5,595,898, 5,576,195, 5,698,435, 5,693,493, 5,698,417, assigned to Xoma, Colligan, supra; Ausubel, supra; or Sambrook, supra, each of the above patents and publications entirely incorporated herein by reference.

Antibodies of the present invention can also be prepared using at least one anti-TNF antibody encoding nucleic acid to provide transgenic animals or mammals, such as goats, cows, horses, sheep, and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

Antibodies of the present invention can additionally be prepared using at least one anti-TNF antibody encoding nucleic acid to provide transgenic plants and cultured plant cells (e.g., but not limited to tobacco and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize have been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. antibodies have also been produced in large amounts from transgenic plant seeds including antibody fragments, such as single chain antibodies (scFv's), including tobacco seeds and potato tubers. See, e.g., Conrad et al., Plant Mol. Biol. 38:101-109 (1998) and reference cited therein. Thus, antibodies of the present invention can also be produced using transgenic plants, according to know methods. See also, e.g., Fischer et al., Biotechnol. Appl. Biochem. 30:99-108 (October 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. Each of the above references is entirely incorporated herein by reference.

The antibodies of the invention can bind human TNF with a wide range of affinities (K_{D}). In a preferred embodiment, at least one human mAb of the present invention can optionally bind human TNF with high affinity. For example, a human mAb can bind human TNF with a K_{D} equal to or less than about 10⁻⁷ M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³ or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer, such as the buffer described herein.

Nucleic Acid Molecules. Using the information provided herein, such as the nucleotide sequences encoding at least 70-100% of the contiguous amino acids of at least one of SEQ ID NOS:1, 2, 3, 4, 5, 6, 7, 8, specified fragments, variants or consensus sequences thereof, or a deposited vector comprising at least one of these sequences, a nucleic acid molecule of the present invention encoding at least one anti-TNF antibody comprising all of the heavy chain variable CDR regions of SEQ ID NOS:1, 2 and 3 and/or all of the light chain variable CDR regions of SEQ ID NOS:4, 5 and 6 can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the present invention can include nucleic acid molecules comprising an open reading frame (ORF), optionally with one or more introns, e.g., but not limited to, at least one specified portion of at least one CDR, as CDR1, CDR2 and/or CDR3 of at least one heavy chain (e.g., SEQ ID NOS:1-3) or light chain (e.g., SEQ ID NOS: 4-6); nucleic acid molecules comprising the coding sequence for an anti-TNF antibody or variable region (e.g., SEQ ID NOS:7,8); and nucleic acid molecules which comprise a nucleotide sequence substantially different from those described above but which, due to the degeneracy of the genetic code, still encode at least one anti-TNF antibody as described herein and/or as known in the art. Of course, the genetic code is well known in the art. Thus, it would be routine for one skilled in the art to generate such degenerate nucleic acid variants that code for specific anti-TNF antibodies of the present invention. See, e.g., Ausubel, et al., supra, and such nucleic acid variants are included in the present invention. Non-limiting examples of isolated nucleic acid molecules of the present invention include SEQ ID NOS:10, 11, 12, 13, 14, 15, corresponding to non-limiting examples of a nucleic acid encoding, respectively, HC CDR1, HC CDR2, HC CDR3, LC CDR1, LC CDR2, LC CDR3, HC variable region and LC variable region.

As indicated herein, nucleic acid molecules of the present invention which comprise a nucleic acid encoding an anti-TNF antibody can include, but are not limited to, those encoding the amino acid sequence of an antibody fragment, by itself; the coding sequence for the entire antibody or a portion thereof; the coding sequence for an antibody, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example—ribosome binding and stability of mRNA); an additional coding sequence that codes for additional amino acids, such as those that provide additional functionalities. Thus, the sequence encoding an antibody can be fused to a marker sequence, such as a sequence encoding a peptide that facilitates purification of the fused antibody comprising an antibody fragment or portion.

Polynucleotides Which Selectively Hybridize to a Polynucleotide as Described Herein. The present invention provides isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Preferably, the cDNA library comprises at least 80% full-length sequences, preferably at least 85% or 90% full-length sequences, and more preferably at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides of this invention will encode at least a portion of an antibody encoded by the polynucleotides described herein. The polynucleotides of this invention embrace nucleic acid sequences that can be employed for selective hybridization to a polynucleotide encoding an antibody of the present invention. See, e.g., Ausubel, supra; Colligan, supra, each entirely incorporated herein by reference.

Construction of Nucleic Acids. The isolated nucleic acids of the present invention can be made using (a) recombinant methods, (b) synthetic techniques, (c) purification techniques, or combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention—excluding the coding sequence—is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

Recombinant Methods for Constructing Nucleic Acids. The isolated nucleic acid compositions of this invention, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, is well known to those of ordinary skill in the art. (See, e.g., Ausubel, supra; or Sambrook, supra).

Nucleic Acid Screening and Isolation Methods. A cDNA or genomic library can be screened using a probe based upon the sequence of a polynucleotide of the present invention, such as those disclosed herein. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. Nos. 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the trade name NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the present invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

Synthetic Methods for Constructing Nucleic Acids. The isolated nucleic acids of the present invention can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

Recombinant Expression Cassettes. The present invention further provides recombinant expression cassettes comprising a nucleic acid of the present invention. A nucleic acid sequence of the present invention, for example a cDNA or a genomic sequence encoding an antibody of the present invention, can be used to construct a recombinant expression cassette that can be introduced into at least one desired host cell. A recombinant expression cassette will typically comprise a polynucleotide of the present invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the present invention.

In some embodiments, isolated nucleic acids that serve as promoter, enhancer, or other elements can be introduced in the appropriate position (upstream, downstream or in intron) of a non-heterologous form of a polynucleotide of the present invention so as to up or down regulate expression of a polynucleotide of the present invention. For example, endogenous promoters can be altered in vivo or in vitro by mutation, deletion and/or substitution.

Vectors and Host Cells. The present invention also relates to vectors that include isolated nucleic acid molecules of the present invention, host cells that are genetically engineered with the recombinant vectors, and the production of at least one anti-TNF antibody by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating site at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be affected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one antibody of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the present invention.

Alternatively, nucleic acids of the present invention can be expressed in a host cell by turning on (by manipulation) in a host cell that contains endogenous DNA encoding an antibody of the present invention. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. Preferred host cells include CHO cells and cells of lymphoid origin such as myeloma and lymphoma cells. Particularly preferred host cells are CHO cells, P3X63Ag8.653 cells (ATCC Accession Number CRL-1580), and SP2/0-Ag14 cells (ATCC Accession Number CRL-1851).

Expression vectors for these cells can include one or more of the following expression control sequences, such as, but not limited to an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

Purification of an Antibody. An anti-TNF antibody can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Antibodies of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody of the present invention can be glycosylated or can be non-glycosylated, with glycosylated preferred. Such methods are described in many standard laboratory manuals, such as Sambrook, supra; Ausubel, supra, Chapters 10, 12, 13, 16, 18 and 20, Colligan, Protein Science, supra, Chapters 12-14, all entirely incorporated herein by reference.

### Exemplary Anti-TNF Antibodies

The isolated antibodies of the present invention, comprising all of the heavy chain variable CDR regions of SEQ ID NOS:1, 2 and 3 and/or all of the light chain variable CDR regions of SEQ ID NOS:4, 5 and 6, comprise antibody amino acid sequences disclosed herein encoded by any suitable polynucleotide, or any isolated or prepared antibody. Preferably, the human antibody or antigen-binding fragment binds human TNF and, thereby partially or substantially neutralizes at least one biological activity of the protein. An antibody, or specified portion or variant thereof, that partially or preferably substantially neutralizes at least one biological activity of at least one TNF protein or fragment can bind the protein or fragment and thereby inhibit activities mediated through the binding of TNF to the TNF receptor or through other TNF-dependent or mediated mechanisms. As used herein, the term “neutralizing antibody” refers to an antibody that can inhibit an TNF-dependent activity by about 20-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an anti-TNF antibody to inhibit an TNF-dependent activity is preferably assessed by at least one suitable TNF protein or receptor assay, as described herein and/or as known in the art. A human antibody of the invention can be of any class (IgG, IgA, IgM, IgE, IgD, etc.) or isotype and can comprise a kappa or lambda light chain. In one embodiment, the human antibody comprises an IgG heavy chain or defined fragment, for example, at least one of isotypes, IgG1, IgG2, IgG3 or IgG4. Antibodies of this type can be prepared by employing a transgenic mouse or other transgenic non-human mammal comprising at least one human light chain (e.g., IgG, IgA) and IgM (e.g., γ1, γ2, γ3, γ4) transgenes as described herein and/or as known in the art. In another embodiment, the anti-human TNF human antibody comprises an IgG1 heavy chain and a IgG1 light chain.

As used herein, the terms “antibody” or “antibodies”, include biosimilar antibody molecules approved under the Biologics Price Competition and Innovation Act of 2009 (BPCI Act) and similar laws and regulations globally. Under the BPCI Act, an antibody may be demonstrated to be biosimilar if data show that it is “highly similar” to the reference product notwithstanding minor differences in clinically inactive components and are “expected” to produce the same clinical result as the reference product in terms of safety, purity and potency (Endocrine Practice: February 2018, Vol. 24, No. 2, pp. 195-204). These biosimilar antibody molecules are provided an abbreviated approval pathway, whereby the applicant relies upon the innovator reference product's clinical data to secure regulatory approval. Compared to the original innovator reference antibody that was FDA approved based on successful clinical trials, a biosimilar antibody molecule is referred to herein as a “follow-on biologic”. As presented herein, SIMPONI® (golimumab) is the original innovator reference anti-TNF antibody that was FDA approved based on successful clinical trials. Golimumab has been on sale in the United States since 2009.

### Example Sequences

In various embodiments, the TNF inhibitor comprises the anti-TNF antibody SIMPONI® (golimumab), or an antigen-binding fragment thereof comprising the sequences shown below. For more information about the anti-TNF antibody SIMPONI® (golimumab) and other anti-TNF antibodies, see e.g., U.S. Pat. Nos.: 7,250,165; 7,691,378; 7,521,206; 7,815,909; 7,820,169; 8,241,899; 8,603,778; 9,321,836; and 9,828,424.

### Example Anti-TNF Antibody Sequences, e.g., SIMPONI® (Golimumab)

Heavy chain CDRs (HCDRs) and light chain CDRs (LCDRs) are defined by Kabat.

| Amino acid sequence ofgolimumab heavy chain (HC) with CDRs underlined: | | |
|---|---|---|
| (SEQ ID NO: 36 | | |
| 1 | QVQLVESGGG VVQPGRSLRL SCAASGFIFS SYAMHWVRQA PGNGLEWVAF MSYDGSNKKY | |
| 61 | ADSVKGRFTI SRDNSKNTLY LQMNSLRAED TAVYYCARDR GIAAGGNYYY YGMDVWGQGT | |
| 121 | TVTVSSASTK GPSVFPLAPS SKSTSGGTAA LGCLVKDYFP EPVTVSWNSG ALTSGVHTFP | |
| 181 | AVLQSSGLYS LSSVVTVPSS SLGTQTYICN VNHKPSNTKV DKKVEPKSCD KTHTCPPCPA | |
| 241 | PELLGGPSVF LFPPKPKDTL MISRTPEVTC VVVDVSHEDP EVKFNWYVDG VEVHNAKTKP | |
| 301 | REEQYNSTYR VVSVLTVLHQ DWLNGKEYKC KVSNKALPAP IEKTISKAKG QPREPQVYTL | |
| 361 | PPSRDELTKN QVSLTCLVKG FYPSDIAVEW ESNGQPENNY KTTPPVLDSD GSFFLYSKLT | |
| 421 | VDKSRWQQGN VFSCSVMHEA LHNHYTQKSL SLSPGK | 456 |

| Amino acid sequence of golimumab light chain (LC) with CDRs underlined: | |
|---|---|
| (SEQ ID NO: 37) | |
| 1 | EIVLTQSPAT LSLSPGERAT LSCRASQSVY SYLAWYQQKP GQAPRLLIYD ASNRATGIPA |
| 61 | RFSGSGSGTD FTLTISSLEP EDFAVYYCQQ RSNWPPFTFG PGTKVDIKRT VAAPSVFIFP |
| 121 | PSDEQLKSGT ASVVCLLNNF YPREAKVQWK VDNALQSGNS QESVTEQDSK DSTYSLSSTL |
| 181 | TLSKADYEKH KVYACEVTHQ GLSSPVTKSF NRGEC |
| Amino acid sequence of golimumab variable heavy chain (VH) with CDRs | |
| underlined: | |
| (SEQ ID NO: 38) | |
| 1 | QVQLVESGGG VVQPGRSLRL SCAASGFIFS SYAMHWVRQA PGNGLEWVAF MSYDGSNKKY |
| 61 | ADSVKGRFTI SRDNSKNTLY LQMNSLRAED TAVYYCARDR GIAAGGNYYY YGMDVWGQGT |
| 121 | TVTVSS |
| Amino acid sequence of golimumab variable light chain (VL) with CDRs | |
| underlined: | |
| (SEQ ID NO: 39) | |
| 1 | EIVLTQSPAT LSLSPGERAT LSCRASQSVY SYLAWYQQKP GQAPRLLIYD ASNRATGIPA |
| 61 | RFSGSGSGTD FTLTISSLEP EDFAVYYCQQ RSNWPPFTFG PGTKVDIKRT V |

Amino acid sequence of golimumab heavy chain complementarity determining
region 1 (HCDR1):
(SEQ ID NO: 40)
SYAMH
Amino acid sequence of golimumab antibody heavy chain complementarity
determining region 2 (HCDR2):
(SEQ ID NO: 41)
FMSYDGSNKKYADSVKG
Amino acid sequence of golimumab heavy chain complementarity determining
region 3 (HCDR3):
(SEQ ID NO: 42)
DRGIAAGGNYYYYGMDV
Amino acid sequence of golimumab light chain complementarity determining
region 1 (LCDR1):
(SEQ ID NO: 43)
RASQSVYSYLA
Amino acid sequence of golimumab light chain complementarity determining
region 2 (LCDR2):
(SEQ ID NO: 44)
DASNRAT
Amino acid sequence of golimumab light chain complementarity determining
region 3 (LCDRL):
(SEQ ID NO: 45)
QQRSNWPPFT

At least one antibody of the invention binds at least one specified epitope specific to at least one TNF protein, subunit, fragment, portion or any combination thereof. The at least one epitope can comprise at least one antibody binding region that comprises at least one portion of said protein, which epitope is preferably comprised of at least one extracellular, soluble, hydrophilic, external or cytoplasmic portion of said protein. The at least one specified epitope can comprise any combination of at least one amino acid sequence of at least 1-3 amino acids to the entire specified portion of contiguous amino acids of the SEQ ID NO:9.

Generally, the human antibody or antigen-binding fragment of the present invention will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region. As a non-limiting example, the antibody or antigen-binding portion or variant can comprise at least one of the heavy chain CDR3 having the amino acid sequence of SEQ ID NO:3, and/or a light chain CDR3 having the amino acid sequence of SEQ ID NO:6. In a particular embodiment, the antibody or antigen-binding fragment can have an antigen-binding region that comprises at least a portion of at least one heavy chain CDR (i.e., CDR1, CDR2 and/or CDR3) having the amino acid sequence of the corresponding CDRs 1, 2 and/or 3 (e.g., SEQ ID NOS:1, 2, and/or 3). In another particular embodiment, the antibody or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3 (e.g., SEQ ID NOS: 4, 5, and/or 6). In a preferred embodiment the three heavy chain CDRs and the three light chain CDRs of the antibody or antigen-binding fragment have the amino acid sequence of the corresponding CDR of at least one of mAb TNV148, TNV14, TNV15, TNV196, TNV118, TNV32, TNV86, as described herein. Such antibodies can be prepared by chemically joining together the various portions (e.g., CDRs, framework) of the antibody using conventional techniques, by preparing and expressing a (i.e., one or more) nucleic acid molecule that encodes the antibody using conventional techniques of recombinant DNA technology or by using any other suitable method.

The anti-TNF antibody can comprise at least one of a heavy or light chain variable region having a defined amino acid sequence. For example, in a preferred embodiment, the anti-TNF antibody comprises at least one of heavy chain variable region, optionally having the amino acid sequence of SEQ ID NO:7 and/or at least one light chain variable region, optionally having the amino acid sequence of SEQ ID NO:8. antibodies that bind to human TNF and that comprise a defined heavy or light chain variable region can be prepared using suitable methods, such as phage display (Katsube, Y., et al., Int J Mol. Med, 1(5):863-868 (1998)) or methods that employ transgenic animals, as known in the art and/or as described herein. For example, a transgenic mouse, comprising a functionally rearranged human immunoglobulin heavy chain transgene and a transgene comprising DNA from a human immunoglobulin light chain locus that can undergo functional rearrangement, can be immunized with human TNF or a fragment thereof to elicit the production of antibodies. If desired, the antibody producing cells can be isolated and hybridomas or other immortalized antibody-producing cells can be prepared as described herein and/or as known in the art. Alternatively, the antibody, specified portion or variant can be expressed using the encoding nucleic acid or portion thereof in a suitable host cell.

The invention also relates to antibodies, antigen-binding fragments, immunoglobulin chains and CDRs comprising amino acids in a sequence that is substantially the same as an amino acid sequence described herein. Preferably, such antibodies or antigen-binding fragments and antibodies comprising such chains or CDRs can bind human TNF with high affinity (e.g., K_{D} less than or equal to about 10⁻⁹ M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/ hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

Amino Acid Codes. The amino acids that make up anti-TNF antibodies of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994):

| SINGLE | THREE | | THREE |
|---|---|---|---|
| LETTER | LETTER | | NUCLEOTIDE |
| CODE | CODE | NAME | CODON(S) |
| A | Ala | Alanine | GCA, GCC, GCG, |
| | | | GCU |
| C | Cys | Cysteine | UGC, UGU |
| D | Asp | Aspartic acid | GAC, GAU |
| E | Glu | Glutamic acid | GAA, GAG |
| F | Phe | Phenylanine | UUC, UUU |
| G | Gly | Glycine | GGA, GGC, GGG, |
| | | | GGU |
| H | His | Histidine | CAC, CAU |
| I | Ile | Isoleucine | AUA, AUC, AUU |
| K | Lys | Lysine | AAA, AAG |
| L | Leu | Leucine | UUA, UUG, CUA, |
| | | | CUC, CUG, CUU |
| M | Met | Methionine | AUG |
| N | Asn | Asparagine | AAC, AAU |
| P | Pro | Proline | CCA, CCC, CCG, CCU |
| Q | Gln | Glutamine | CAA, CAG |
| R | Arg | Arginine | AGA, AGG, CGA, |
| | | | CGC, CGG, CGU |
| S | Ser | Serine | AGC, AGU, UCA, |
| | | | UCC, UCG, UCU |
| T | Thr | Threonine | ACA, ACC, ACG, |
| | | | ACU |
| V | Val | Valine | GUA, GUC, GUG, |
| | | | GUU |
| W | Trp | Tryptophan | UGG |
| Y | Tyr | Tyrosine | UAC, UAU |

An anti-TNF antibody of the present invention can include one or more amino acid substitutions, deletions or additions, either from natural mutations or human manipulation, as specified herein.

Of course, the number of amino acid substitutions a skilled artisan would make depends on many factors, including those described above. Generally speaking, the number of amino acid substitutions, insertions or deletions for any given anti-TNF antibody, fragment or variant will not be more than 40, 30, 20, 19, 18, 17, 16, 15, 14, 13, 12, 11, 10, 9, 8, 7, 6, 5, 4, 3, 2, 1, such as 1-30 or any range or value therein, as specified herein.

Amino acids in an anti-TNF antibody of the present invention that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to at least one TNF neutralizing activity. Sites that are critical for antibody binding can also be identified by structural analysis such as crystallization, nuclear magnetic resonance or photoaffinity labeling (Smith, et al., J. Mol. Biol. 224:899-904 (1992) and de Vos, et al., Science 255:306-312 (1992)).

Anti-TNF antibodies of the present invention can include, but are not limited to, at least one portion, sequence or combination selected from 1 to all of the contiguous amino acids of at least one of SEQ ID NOS:1, 2, 3, 4, 5, 6.

A(n) anti-TNF antibody can further optionally comprise a polypeptide of at least one of 70-100% of the contiguous amino acids of at least one of SEQ ID NOS:7, 8.

In one embodiment, the amino acid sequence of an immunoglobulin chain, or portion thereof (e.g., variable region, CDR) has about 70-100% identity (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or any range or value therein) to the amino acid sequence of the corresponding chain of at least one of SEQ ID NOS:7, 8. For example, the amino acid sequence of a light chain variable region can be compared with the sequence of SEQ ID NO: 8, or the amino acid sequence of a heavy chain CDR3 can be compared with SEQ ID NO:7. Preferably, 70-100% amino acid identity (i.e., 90, 91, 92, 93, 94, 95, 96, 97, 98, 99, 100 or any range or value therein) is determined using a suitable computer algorithm, as known in the art.

Exemplary heavy chain and light chain variable regions sequences are provided in SEQ ID NOS: 7, 8. The antibodies of the present invention, or specified variants thereof, can comprise any number of contiguous amino acid residues from an antibody of the present invention, wherein that number is selected from the group of integers consisting of from 10-100% of the number of contiguous residues in an anti-TNF antibody. Optionally, this subsequence of contiguous amino acids is at least about 10, 20, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, or 5.

As those of skill will appreciate, the present invention includes at least one biologically active antibody of the present invention. Biologically active antibodies have a specific activity at least 20%, 30%, or 40%, and preferably at least 50%, 60%, or 70%, and most preferably at least 80%, 90%, or 95%-100% of that of the native (non-synthetic), endogenous or related and known antibody. Methods of assaying and quantifying measures of enzymatic activity and substrate specificity, are well known to those of skill in the art.

In another aspect, the invention relates to human antibodies and antigen-binding fragments, as described herein, which are modified by the covalent attachment of an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified antibodies and antigen-binding fragments of the invention can comprise one or more organic moieties that are covalently bonded, directly or indirectly, to the antibody. Each organic moiety that is bonded to an antibody or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an antibody modified by the covalent attachment of polylysine is encompassed by the invention. Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody of the invention has a molecular weight of about 800 to about 150,000 Daltons as a separate molecular entity. For example, PEG₅₀₀₀ and PEG_{20,000}, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Fatty acids that are suitable for modifying antibodies of the invention include, for example, n-dodecanoate (C₁₂, laurate), n-tetradecanoate (C₁₄, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), cis-Δ9-octadecanoate (C₁₈, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The modified human antibodies and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example a divalent C₁-C₁₂ group wherein one or more carbon atoms can be replaced by a heteroatom such as oxygen, nitrogen or sulfur. Suitable linker moieties include, for example, tetraethylene glycol, —(CH₂)₃—, —NH—(CH₂)₆—NH—, —(CH₂)₂—NH— and —CH₂—O—CH₂—CH₂—O—CH₂—CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described or can be reacted with maleic anhydride and the resulting product cyclized to produce an activated maleimido derivative of the fatty acid. (See, for example, Thompson, et al., WO 92/16221 the entire teachings of which are incorporated herein by reference.)

The modified antibodies of the invention can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

Anti-Idiotype Antibodies To Anti-Tnf Antibody Compositions. In addition to monoclonal or chimeric anti-TNF antibodies, the present invention is also directed to an anti-idiotypic (anti-Id) antibody specific for such antibodies of the invention. An anti-Id antibody is an antibody which recognizes unique determinants generally associated with the antigen-binding region of another antibody. The anti-Id can be prepared by immunizing an animal of the same species and genetic type (e.g. mouse strain) as the source of the Id antibody with the antibody or a CDR containing region thereof. The immunized animal will recognize and respond to the idiotypic determinants of the immunizing antibody and produce an anti-Id antibody. The anti-Id antibody may also be used as an “immunogen” to induce an immune response in yet another animal, producing a so-called anti-anti-Id antibody.

Anti-Tnf Antibody Compositions. The present invention also provides at least one anti-TNF antibody composition comprising at least one, at least two, at least three, at least four, at least five, at least six or more anti-TNF antibodies thereof, as described herein and/or as known in the art that are provided in a non-naturally occurring composition, mixture or form. Such compositions comprise non-naturally occurring compositions comprising at least one or two full length, C- and/or N-terminally deleted variants, domains, fragments, or specified variants, of the anti-TNF antibody amino acid sequence selected from the group consisting of 70-100% of the contiguous amino acids of SEQ ID NOS:1, 2, 3, 4, 5, 6, 7, 8, or specified fragments, domains or variants thereof Preferred anti-TNF antibody compositions include at least one or two full length, fragments, domains or variants as at least one CDR or LBR containing portions of the anti-TNF antibody sequence of 70-100% of SEQ ID NOS:1, 2, 3, 4, 5, 6, or specified fragments, domains or variants thereof. Further preferred compositions comprise 40-99% of at least one of 70-100% of SEQ ID NOS:1, 2, 3, 4, 5, 6, or specified fragments, domains or variants thereof. Such composition percentages are by weight, volume, concentration, molarity, or molality as liquid or dry solutions, mixtures, suspension, emulsions or colloids, as known in the art or as described herein.

Anti-TNF antibody compositions of the present invention can further comprise at least one of any suitable and effective amount of a composition or pharmaceutical composition comprising at least one anti-TNF antibody to a cell, tissue, organ, animal or patient in need of such modulation, treatment or therapy, optionally further comprising at least one selected from at least one TNF antagonist (e.g., but not limited to a TNF antibody or fragment, a soluble TNF receptor or fragment, fusion proteins thereof, or a small molecule TNF antagonist), an antirheumatic (e.g., methotrexate, auranofin, aurothioglucose, azathioprine, etanercept, gold sodium thiomalate, hydroxychloroquine sulfate, leflunomide, sulfasalazine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a fluoroquinolone, a macrolide, a penicillin, a sulfonamide, a tetracycline, another antimicrobial), an antipsoriatic, a corticosteroid, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Non-limiting examples of such cytokines include, but are not limited to, any of IL-1 to IL-23. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

Such anti-cancer or anti-infectives can also include toxin molecules that are associated, bound, co-formulated or co-administered with at least one antibody of the present invention. The toxin can optionally act to selectively kill the pathologic cell or tissue. The pathologic cell can be a cancer or other cell. Such toxins can be, but are not limited to, purified or recombinant toxin or toxin fragment comprising at least one functional cytotoxic domain of toxin, e.g., selected from at least one of ricin, diphtheria toxin, a venom toxin, or a bacterial toxin. The term toxin also includes both endotoxins and exotoxins produced by any naturally occurring, mutant or recombinant bacteria or viruses which may cause any pathological condition in humans and other mammals, including toxin shock, which can result in death. Such toxins may include, but are not limited to, enterotoxigenic E. coli heat-labile enterotoxin (LT), heat-stable enterotoxin (ST), Shigella cytotoxin, Aeromonas enterotoxins, toxic shock syndrome toxin-1 (TSST-1), Staphylococcal enterotoxin A (SEA), B (SEB), or C (SEC), Streptococcal enterotoxins and the like. Such bacteria include, but are not limited to, strains of a species of enterotoxigenic E. coli (ETEC), enterohemorrhagic E. coli (e.g., strains of serotype 0157:H7), Staphylococcus species (e.g., Staphylococcus aureus, Staphylococcus pyogenes), Shigella species (e.g., Shigella dysenteriae, Shigella flexneri, Shigella boydii, and Shigella sonnei), Salmonella species (e.g., Salmonella typhi, Salmonella cholera-suis, Salmonella enteritidis), Clostridium species (e.g., Clostridium perfringens, Clostridium difficile, Clostridium botulinum), Camphlobacter species (e.g., Camphlobacter jejuni, Camphlobacter fetus), Heliocbacter species, (e.g., Heliocbacter pylori), Aeromonas species (e.g., Aeromonas sobria, Aeromonas hydrophila, Aeromonas caviae), Pleisomonas shigelloides, Yersinia enterocolitica, Vibrio species (e.g., Vibrio cholerae, Vibrio parahemolyticus), Klebsiella species, Pseudomonas aeruginosa, and Streptococci. See, e.g., Stein, ed., INTERNAL MEDICINE, 3rd ed., pp 1-13, Little, Brown and Co., Boston, (1990); Evans et al., eds., Bacterial Infections of Humans: Epidemiology and Control, 2d. Ed., pp 239-254, Plenum Medical Book Co., New York (1991); Mandell et al, Principles and Practice of Infectious Diseases, 3d. Ed., Churchill Livingstone, New York (1990); Berkow et al, eds., The Merck Manual, 16th edition, Merck and Co., Rahway, N.J., 1992; Wood et al, FEMS Microbiology Immunology, 76:121-134 (1991); Marrack et al, Science, 248:705-711 (1990), the contents of which references are incorporated entirely herein by reference.

Anti-TNF antibody compounds, compositions or combinations of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, Pa.) 1990. Pharmaceutically acceptable carriers can be routinely selected that are suitable for the mode of administration, solubility and/or stability of the anti-TNF antibody, fragment or variant composition as well known in the art or as described herein.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Anti-TNF antibody compositions can also include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts such as citrate.

Additionally, anti-TNF antibody compositions of the invention can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the anti-TNF antibody, portion or variant compositions according to the invention are known in the art, e.g., as listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), the disclosures of which are entirely incorporated herein by reference. Preferred carrier or excipient materials are carbohydrates (e.g., saccharides and alditols) and buffers (e.g., citrate) or polymeric agents.

Formulations. As noted above, the invention provides for stable formulations, which is preferably a phosphate buffer with saline or a chosen salt, as well as preserved solutions and formulations containing a preservative as well as multi-use preserved formulations suitable for pharmaceutical or veterinary use, comprising at least one anti-TNF antibody in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1., 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the invention provides an article of manufacture, comprising packaging material and at least one vial comprising a solution of at least one anti-TNF antibody with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising lyophilized at least one anti-TNF antibody, and a second vial comprising an aqueous diluent of prescribed buffer or preservative, wherein said packaging material comprises a label that instructs a patient to reconstitute the at least one anti-TNF antibody in the aqueous diluent to form a solution that can be held over a period of twenty-four hours or greater.

The at least one anti-TNF antibody used in accordance with the present invention can be produced by recombinant means, including from mammalian cell or transgenic preparations, or can be purified from other biological sources, as described herein or as known in the art.

The range of at least one anti-TNF antibody in the product of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g. isotonicity agents, buffers, antioxidants, preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably the formulations of the present invention have pH between about 6.8 and about 7.8. Preferred buffers include phosphate buffers, most preferably sodium phosphate, particularly phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, Pluronic® polyols, other block co-polymers, and chelators such as EDTA and EGTA can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations of the present invention can be prepared by a process which comprises mixing at least one anti-TNF antibody and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one anti-TNF antibody and preservative in an aqueous diluent is carried out using conventional dissolution and mixing procedures. To prepare a suitable formulation, for example, a measured amount of at least one anti-TNF antibody in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed formulations can be provided to patients as clear solutions or as dual vials comprising a vial of lyophilized at least one anti-TNF antibody that is reconstituted with a second vial containing water, a preservative and/or excipients, preferably a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present claimed articles of manufacture are useful for administration over a period of immediately to twenty-four hours or greater. Accordingly, the presently claimed articles of manufacture offer significant advantages to the patient. Formulations of the invention can optionally be safely stored at temperatures of from about 2 to about 40° C. and retain the biologically activity of the protein for extended periods of time, thus, allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of at least one anti-TNF antibody in the invention can be prepared by a process that comprises mixing at least one antibody in an aqueous diluent. Mixing is carried out using conventional dissolution and mixing procedures. To prepare a suitable diluent, for example, a measured amount of at least one antibody in water or buffer is combined in quantities sufficient to provide the protein and optionally a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to patients as clear solutions or as dual vials comprising a vial of lyophilized at least one anti-TNF antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one anti-TNF antibody that is reconstituted with a second vial containing the aqueous diluent. The clear solution in this case can be up to one liter or even larger in size, providing a large reservoir from which smaller portions of the at least one antibody solution can be retrieved one or multiple times for transfer into smaller vials and provided by the pharmacy or clinic to their customers and/or patients.

Recognized devices comprising these single vial systems include those pen-injector devices for delivery of a solution such as B-D® (pen injector device), NOVOPEN® (pen injector device), AUTOPEN® (pen injector device), OPTIPEN® (pen injector device), GENOTROPIN PEN® (pen injector device),-HUMATROPEN® (pen injector device), BIOJECTOR® (pen injector device), Reco-Pen, Humaject, J-tip Needle-Free Injector, Intraject, Medi-Ject, e.g., as made or developed by:

Becton Dickensen (Franklin Lakes, N.J., www.bectondickenson.com),

Disetronic (Burgdorf, Switzerland, www.disetronic.com;

Bioject, Portland, Oreg. (www.bioject.com);

Weston Medical (Peterborough, UK, www.weston-medical.com),

Medi-Ject Corp (Minneapolis, Minn., www.mediject.com).

Recognized devices comprising a dual vial system include those pen-injector systems for reconstituting a lyophilized drug in a cartridge for delivery of the reconstituted solution such as the HUMATROPEN® (pen injector device)

The products presently claimed include packaging material. The packaging material provides, in addition to the information required by the regulatory agencies, the conditions under which the product can be used. The packaging material of the present invention provides instructions to the patient to reconstitute the at least one anti-TNF antibody in the aqueous diluent to form a solution and to use the solution over a period of 2-24 hours or greater for the two vial, wet/dry, product. For the single vial, solution product, the label indicates that such solution can be used over a period of 2-24 hours or greater. The presently claimed products are useful for human pharmaceutical product use.

The formulations of the present invention can be prepared by a process that comprises mixing at least one anti-TNF antibody and a selected buffer, preferably a phosphate buffer containing saline or a chosen salt. Mixing the at least one antibody and buffer in an aqueous diluent is carried out using conventional dissolution and mixing procedures. To prepare a suitable formulation, for example, a measured amount of at least one antibody in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed stable or preserved formulations can be provided to patients as clear solutions or as dual vials comprising a vial of lyophilized at least one anti-TNF antibody that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

At least one anti-TNF antibody in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

Therapeutic Applications. The present invention also provides a method for modulating or treating at least one TNF related disease, in a cell, tissue, organ, animal, or patient, as known in the art or as described herein, using at least one dual integrin antibody of the present invention.

The present invention also provides a method for modulating or treating at least one TNF related disease, in a cell, tissue, organ, animal, or patient including, but not limited to, at least one of obesity, an immune related disease, a cardiovascular disease, an infectious disease, a malignant disease or a neurologic disease.

The present invention also provides a method for modulating or treating at least one immune related disease, in a cell, tissue, organ, animal, or patient including, but not limited to, at least one of rheumatoid arthritis, juvenile, systemic onset juvenile rheumatoid arthritis, Ankylosing Spondylitis, ankylosing spondylitis, gastric ulcer, seronegative arthropathies, osteoarthritis, inflammatory bowel disease, ulcerative colitis, systemic lupus erythematosus, antiphospholipid syndrome, iridocyclitis/uveitis/optic neuritis, idiopathic pulmonary fibrosis, systemic vasculitis/wegener's granulomatosis, sarcoidosis, orchitis/vasectomy reversal procedures, allergic/atopic diseases, asthma, allergic rhinitis, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, burns, ionizing radiation exposure, acute pancreatitis, adult respiratory distress syndrome, alcohol-induced hepatitis, chronic inflammatory pathologies, sarcoidosis, Crohn's pathology, sickle cell anemia, diabetes, nephrosis, atopic diseases, hypersensitivity reactions, allergic rhinitis, hay fever, perennial rhinitis, conjunctivitis, endometriosis, asthma, urticaria, systemic anaphylaxis, dermatitis, pernicious anemia, hemolytic disease, thrombocytopenia, graft rejection of any organ or tissue, kidney transplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Graves disease, Raynoud's disease, type B insulin-resistant diabetes, asthma, myasthenia gravis, antibody-meditated cytotoxicity, type III hypersensitivity reactions, systemic lupus erythematosus, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, scleroderma, mixed connective tissue disease, idiopathic Addison's disease, diabetes mellitus, chronic active hepatitis, primary biliary cirrhosis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic/idiopathic, Wilson's disease, hemochromatosis, alpha-1-antitrypsin deficiency, diabetic retinopathy, hashimoto's thyroiditis, osteoporosis, primary biliary cirrhosis, thyroiditis, encephalomyelitis, cachexia, cystic fibrosis, neonatal chronic lung disease, chronic obstructive pulmonary disease (COPD), familial hemophagocytic lymphohistiocytosis, dermatologic conditions, psoriasis, alopecia, nephrotic syndrome, nephritis, glomerular nephritis, acute renal failure, hemodialysis, uremia, toxicity, preeclampsia, okt3 therapy, anti-cd3 therapy, cytokine therapy, chemotherapy, radiation therapy (e.g., including but not limited to asthenia, anemia, cachexia, and the like), chronic salicylate intoxication, and the like. See, e.g., the Merck Manual, 12th-17th Editions, Merck & Company, Rahway, N.J. (1972, 1977, 1982, 1987, 1992, 1999), Pharmacotherapy Handbook, Wells et al., eds., Second Edition, Appleton and Lange, Stamford, Conn. (1998, 2000), each entirely incorporated by reference.

The present invention also provides a method for modulating or treating at least one cardiovascular disease in a cell, tissue, organ, animal, or patient, including, but not limited to, at least one of cardiac stun syndrome, myocardial infarction, congestive heart failure, stroke, ischemic stroke, hemorrhage, arteriosclerosis, atherosclerosis, restenosis, diabetic arteriosclerotic disease, hypertension, arterial hypertension, renovascular hypertension, syncope, shock, syphilis of the cardiovascular system, heart failure, cor pulmonale, primary pulmonary hypertension, cardiac arrhythmias, atrial ectopic beats, atrial flutter, atrial fibrillation (sustained or paroxysmal), post perfusion syndrome, cardiopulmonary bypass inflammation response, chaotic or multifocal atrial tachycardia, regular narrow QRS tachycardia, specific arrhythmias, ventricular fibrillation, His bundle arrhythmias, atrioventricular block, bundle branch block, myocardial ischemic disorders, coronary artery disease, angina pectoris, myocardial infarction, cardiomyopathy, dilated congestive cardiomyopathy, restrictive cardiomyopathy, valvular heart diseases, endocarditis, pericardial disease, cardiac tumors, aortic and peripheral aneurysms, aortic dissection, inflammation of the aorta, occlusion of the abdominal aorta and its branches, peripheral vascular disorders, occlusive arterial disorders, peripheral atherosclerotic disease, thromboangiitis obliterans, functional peripheral arterial disorders, Raynaud's phenomenon and disease, acrocyanosis, erythromelalgia, venous diseases, venous thrombosis, varicose veins, arteriovenous fistula, lymphedema, lipedema, unstable angina, reperfusion injury, post pump syndrome, ischemia-reperfusion injury, and the like. Such a method can optionally comprise administering an effective amount of a composition or pharmaceutical composition comprising at least one anti-TNF antibody to a cell, tissue, organ, animal or patient in need of such modulation, treatment or therapy.

The present invention also provides a method for modulating or treating at least one infectious disease in a cell, tissue, organ, animal or patient, including, but not limited to, at least one of: acute or chronic bacterial infection, acute and chronic parasitic or infectious processes, including bacterial, viral and fungal infections, HIV infection/HIV neuropathy, meningitis, hepatitis (A,B or C, or the like), septic arthritis, peritonitis, pneumonia, epiglottitis, E. coli 0157:h7, hemolytic uremic syndrome/thrombolytic thrombocytopenic purpura, malaria, dengue hemorrhagic fever, leishmaniasis, leprosy, toxic shock syndrome, streptococcal myositis, gas gangrene, mycobacterium tuberculosis, mycobacterium avium intracellulare, pneumocystis carinii pneumonia, pelvic inflammatory disease, orchitis/epididymitis, Legionella, lyme disease, influenza a, epstein-barr virus, viral-associated hemophagocytic syndrome, vital encephalitis/aseptic meningitis, and the like.

The present invention also provides a method for modulating or treating at least one malignant disease in a cell, tissue, organ, animal or patient, including, but not limited to, at least one of: leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), hairy cell leukemia, myelodysplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, adenocarcinomas, sarcomas, malignant melanoma, hemangioma, metastatic disease, cancer related bone resorption, cancer related bone pain, and the like.

The present invention also provides a method for modulating or treating at least one neurologic disease in a cell, tissue, organ, animal or patient, including, but not limited to, at least one of: neurodegenerative diseases, multiple sclerosis, migraine headache, AIDS dementia complex, demyelinating diseases, such as multiple sclerosis and acute transverse myelitis; extrapyramidal and cerebellar disorders, such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; Progressive supranuclear Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoproteinemia, ataxia, telangiectasia, and mitochondrial multisystem disorder); demyelinating core disorders, such as multiple sclerosis, acute transverse myelitis; and disorders of the motor unit' such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Hallerrorden-Spatz disease; and Dementia pugilistica, and the like. Such a method can optionally comprise administering an effective amount of a composition or pharmaceutical composition comprising at least one TNF antibody or specified portion or variant to a cell, tissue, organ, animal or patient in need of such modulation, treatment or therapy. See, e.g., the Merck Manual, 16^{th} Edition, Merck & Company, Rahway, N.J. (1992)

Any method of the present invention can comprise administering an effective amount of a composition or pharmaceutical composition comprising at least one anti-TNF antibody to a cell, tissue, organ, animal or patient in need of such modulation, treatment or therapy. Such a method can optionally further comprise co-administration or combination therapy for treating such immune diseases, wherein the administering of said at least one anti-TNF antibody, specified portion or variant thereof, further comprises administering, before concurrently, and/or after, at least one selected from at least one TNF antagonist (e.g., but not limited to a TNF antibody or fragment, a soluble TNF receptor or fragment, fusion proteins thereof, or a small molecule TNF antagonist), an antirheumatic (e.g., methotrexate, auranofin, aurothioglucose, azathioprine, etanercept, gold sodium thiomalate, hydroxychloroquine sulfate, leflunomide, sulfasalazine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a fluroquinolone, a macrolide, a penicillin, a sulfonamide, a tetracycline, another antimicrobial), an antipsoriatic, a corticosteroid, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

TNF antagonists suitable for compositions, combination therapy, co-administration, devices and/or methods of the present invention (further comprising at least one anti body, specified portion and variant thereof, of the present invention), include, but are not limited to, anti-TNF antibodies, antigen-binding fragments thereof, and receptor molecules which bind specifically to TNF; compounds which prevent and/or inhibit TNF synthesis, TNF release or its action on target cells, such as thalidomide, tenidap, phosphodiesterase inhibitors (e.g., pentoxifylline and rolipram), A2b adenosine receptor agonists and A2b adenosine receptor enhancers; compounds which prevent and/or inhibit TNF receptor signaling, such as mitogen activated protein (MAP) kinase inhibitors; compounds which block and/or inhibit membrane TNF cleavage, such as metalloproteinase inhibitors; compounds which block and/or inhibit TNF activity, such as angiotensin converting enzyme (ACE) inhibitors (e.g., captopril); and compounds which block and/or inhibit TNF production and/or synthesis, such as MAP kinase inhibitors.

As used herein, a “tumor necrosis factor antibody,” “TNF antibody,” “TNFα antibody,” or fragment and the like decreases, blocks, inhibits, abrogates or interferes with TNFα activity in vitro, in situ and/or preferably in vivo. For example, a suitable TNF human antibody of the present invention can bind TNFα and includes anti-TNF antibodies, antigen-binding fragments thereof, and specified mutants or domains thereof that bind specifically to TNFα. A suitable TNF antibody or fragment can also decrease block, abrogate, interfere, prevent and/or inhibit TNF RNA, DNA or protein synthesis, TNF release, TNF receptor signaling, membrane TNF cleavage, TNF activity, TNF production and/or synthesis.

Chimeric antibody cA2 consists of the antigen binding variable region of the high-affinity neutralizing mouse anti-human TNFα IgG1 antibody, designated A2, and the constant regions of a human IgG1, kappa immunoglobulin. The human IgG1 Fc region improves allogeneic antibody effector function, increases the circulating serum half-life and decreases the immunogenicity of the antibody. The avidity and epitope specificity of the chimeric antibody cA2 is derived from the variable region of the murine antibody A2. In a particular embodiment, a preferred source for nucleic acids encoding the variable region of the murine antibody A2 is the A2 hybridoma cell line.

Chimeric A2 (cA2) neutralizes the cytotoxic effect of both natural and recombinant human TNFα in a dose dependent manner. From binding assays of chimeric antibody cA2 and recombinant human TNFα, the affinity constant of chimeric antibody cA2 was calculated to be 1.04×10¹⁰M⁻¹. Preferred methods for determining monoclonal antibody specificity and affinity by competitive inhibition can be found in Harlow, et al., antibodies: A Laboratory Manual, Cold Spring Harbor Laboratory Press, Cold Spring Harbor, N.Y., 1988; Colligan et al., eds., Current Protocols in Immunology, Greene Publishing Assoc. and Wiley Interscience, New York, (1992-2000); Kozbor et al., Immunol. Today, 4:72-79 (1983); Ausubel et al., eds. Current Protocols in Molecular Biology, Wiley Interscience, New York (1987-2000); and Muller, Meth. Enzymol., 92:589-601 (1983), which references are entirely incorporated herein by reference.

In a particular embodiment, murine monoclonal antibody A2 is produced by a cell line designated c134A. Chimeric antibody cA2 is produced by a cell line designated c168A.

Additional examples of monoclonal anti-TNF antibodies that can be used in the present invention are described in the art (see, e.g., U.S. Pat. No. 5,231,024; Moller, A. et al., Cytokine 2(3):162-169 (1990); U.S. application Ser. No. 07/943,852 (filed Sep. 11, 1992); Rathj en et al., International Publication No. WO 91/02078 (published Feb. 21, 1991); Rubin et al., EPO Patent Publication No. 0 218 868 (published Apr. 22, 1987); Yone et al., EPO Patent Publication No. 0 288 088 (Oct. 26, 1988); Liang, et al., Biochem. Biophys. Res. Comm. 137:847-854 (1986); Meager, et al., Hybridoma 6:305-311 (1987); Fendly et al., Hybridoma 6:359-369 (1987); Bringman, et al., Hybridoma 6:489-507 (1987); and Hirai, et al., J. Immunol. Meth. 96:57-62 (1987), which references are entirely incorporated herein by reference).

TNF Receptor Molecules. Preferred TNF receptor molecules useful in the present invention are those that bind TNFα with high affinity (see, e.g., Feldmann et al., International Publication No. WO 92/07076 (published Apr. 30, 1992); Schall et al., Cell 61:361-370 (1990); and Loetscher et al., Cell 61:351-359 (1990), which references are entirely incorporated herein by reference) and optionally possess low immunogenicity. In particular, the 55 kDa (p55 TNF-R) and the 75 kDa (p75 TNF-R) TNF cell surface receptors are useful in the present invention. Truncated forms of these receptors, comprising the extracellular domains (ECD) of the receptors or functional portions thereof (see, e.g., Corcoran et al., Eur. J. Biochem. 223:831-840 (1994)), are also useful in the present invention. Truncated forms of the TNF receptors, comprising the ECD, have been detected in urine and serum as 30 kDa and 40 kDa TNFα inhibitory binding proteins (Engelmann, H. et al., J. Biol. Chem. 265:1531-1536 (1990)). TNF receptor multimeric molecules and TNF immunoreceptor fusion molecules, and derivatives and fragments or portions thereof, are additional examples of TNF receptor molecules which are useful in the methods and compositions of the present invention. The TNF receptor molecules which can be used in the invention are characterized by their ability to treat patients for extended periods with good to excellent alleviation of symptoms and low toxicity. Low immunogenicity and/or high affinity, as well as other undefined properties, can contribute to the therapeutic results achieved.

TNF receptor multimeric molecules useful in the present invention comprise all or a functional portion of the ECD of two or more TNF receptors linked via one or more polypeptide linkers or other nonpeptide linkers, such as polyethylene glycol (PEG). The multimeric molecules can further comprise a signal peptide of a secreted protein to direct expression of the multimeric molecule. These multimeric molecules and methods for their production have been described in U.S. application Ser. No. 08/437,533 (filed May 9, 1995), the content of which is entirely incorporated herein by reference.

TNF immunoreceptor fusion molecules useful in the methods and compositions of the present invention comprise at least one portion of one or more immunoglobulin molecules and all or a functional portion of one or more TNF receptors. These immunoreceptor fusion molecules can be assembled as monomers, or hetero- or homo-multimers. The immunoreceptor fusion molecules can also be monovalent or multivalent. An example of such a TNF immunoreceptor fusion molecule is TNF receptor/IgG fusion protein. TNF immunoreceptor fusion molecules and methods for their production have been described in the art (Lesslauer et al., Eur. J. Immunol. 21:2883-2886 (1991); Ashkenazi et al., Proc. Natl. Acad. Sci. USA 88:10535-10539 (1991); Peppel et al., J. Exp. Med. 174:1483-1489 (1991); Kolls et al., Proc. Natl. Acad. Sci. USA 91:215-219 (1994); Butler et al., Cytokine 6(6):616-623 (1994); Baker et al., Eur. J. Immunol. 24:2040-2048 (1994); Beutler et al., U.S. Pat. No. 5,447,851; and U.S. application Ser. No. 08/442,133 (filed May 16, 1995), each of which references are entirely incorporated herein by reference). Methods for producing immunoreceptor fusion molecules can also be found in Capon et al., U.S. Pat. No. 5,116,964; Capon et al., U.S. Pat. No. 5,225,538; and Capon et al., Nature 337:525-531 (1989), which references are entirely incorporated herein by reference.

A functional equivalent, derivative, fragment or region of TNF receptor molecule refers to the portion of the TNF receptor molecule, or the portion of the TNF receptor molecule sequence which encodes TNF receptor molecule, that is of sufficient size and sequences to functionally resemble TNF receptor molecules that can be used in the present invention (e.g., bind TNFα with high affinity and possess low immunogenicity). A functional equivalent of TNF receptor molecule also includes modified TNF receptor molecules that functionally resemble TNF receptor molecules that can be used in the present invention (e.g., bind TNFα with high affinity and possess low immunogenicity). For example, a functional equivalent of TNF receptor molecule can contain a “SILENT” codon or one or more amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., Current Protocols in Molecular Biology, Greene Publishing Assoc. and Wiley-Interscience, New York (1987-2000).

Cytokines include any known cytokine. See, e.g., CopewithCytokines.com. Cytokine antagonists include, but are not limited to, any antibody, fragment or mimetic, any soluble receptor, fragment or mimetic, any small molecule antagonist, or any combination thereof.

Therapeutic Treatments. Any method of the present invention can comprise a method for treating a TNF mediated disorder, comprising administering an effective amount of a composition or pharmaceutical composition comprising at least one anti-TNF antibody to a cell, tissue, organ, animal or patient in need of such modulation, treatment or therapy. Such a method can optionally further comprise co-administration or combination therapy for treating such immune diseases, wherein the administering of said at least one anti-TNF antibody, specified portion or variant thereof, further comprises administering, before concurrently, and/or after, at least one selected from at least one TNF antagonist (e.g., but not limited to a TNF antibody or fragment, a soluble TNF receptor or fragment, fusion proteins thereof, or a small molecule TNF antagonist), an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist.

As used herein, the term “safe”, as it relates to a composition, dose, dosage regimen, treatment or method with an anti-TNF antibody of the present invention (e.g., the anti-TNF antibody golimumab), refers to a favorable risk:benefit ratio with an acceptable frequency and/or acceptable severity of adverse events (AEs) and serious adverse events (SAEs) compared to the standard of care or to another comparator such as other anti-TNF agents. An adverse event is an untoward medical occurrence in a patient administered a medicinal product. In particular, safe as it relates to a composition, dose, dosage regimen, treatment or method with an anti-TNF antibody of the present invention refers to an acceptable frequency and/or acceptable severity of adverse events including, for example, infusion reactions, hepatobiliary laboratory abnormalities, infections including TB, and malignancies.

The terms “efficacy” and “effective” as used herein in the context of a composition, dose, dosage regimen, treatment or method refer to the effectiveness of a particular composition, dose, dosage, treatment or method with an anti-TNF antibody of the present invention (e.g., the anti-TNF antibody golimumab). Efficacy can be measured based on change in the course of the disease in response to an agent of the present invention. For example, an anti-TNF antibody of the present invention is administered to a patient in an amount and for a time sufficient to induce an improvement, preferably a sustained improvement, in at least one indicator that reflects the severity of the disorder that is being treated. Various indicators that reflect the extent of the subject's illness, disease or condition may be assessed for determining whether the amount and time of the treatment is sufficient. Such indicators include, for example, clinically recognized indicators of disease severity, symptoms, or manifestations of the disorder in question. The degree of improvement generally is determined by a physician or other adequately trained individual, who may make the determination based on signs, symptoms, biopsies, or other test results that indicate amelioration of clinical symptoms or any other measure of disease activity. For example, an anti-TNF antibody of the present invention may be administered to achieve an improvement in a patient's condition related to juvenile idiopathic arthritis (JIA), and in particular for polyarticular juvenile idiopathic arthritis (pJIA). Efficacy for the treatment of JIA and/or pJIA can be determined, for example by patients meeting the criteria for inactive disease, patients having an improvement from baseline corresponding to a JIA American College of Rheumatology (JIA ACR) response selected from JIA ACR 30, JIA ACR 50, JIA ACR 70, and/or JIA ACR 90, and/or patients having a decrease from baseline in Juvenile Arthritis Disease Activity Score (JADAS) selected from JADAS 10, JADAS 27, and/or JADAS 71.

As used herein, unless otherwise noted, the term “clinically proven” (used independently or to modify the terms “safe” and/or “effective”) shall mean that it has been proven by a clinical trial wherein the clinical trial has met the approval standards of U.S. Food and Drug Administration, EMEA or a corresponding national regulatory agency. For example, the clinical study may be an adequately sized, randomized, double-blinded study used to clinically prove the effects of the drug.

Typically, treatment of pathologic conditions is effected by administering a safe and effective amount or dosage of at least one anti-TNF antibody composition that total, on average, a range from at least about 0.01 to 500 milligrams of at least one anti-TNFantibody per kilogram of patient per dose, and preferably from at least about 0.1 to 100 milligrams antibody /kilogram of patient per single or multiple administration, depending upon the specific activity of contained in the composition. Alternatively, the effective serum concentration can comprise 0.1-5000 μg/ml serum concentration per single or multiple administration. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably 0.1 to 10 milligrams per kilogram per administration or in sustained release form is effective to obtain desired results.

As a non-limiting example, treatment of humans or animals can be provided as a one-time or periodic dosage of at least one antibody of the present invention 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Dosage forms (composition) suitable for internal administration generally contain from about 0.1 milligram to about 500 milligrams of active ingredient per unit or container. In these pharmaceutical compositions the active ingredient will ordinarily be present in an amount of about 0.5-99.999% by weight based on the total weight of the composition.

For parenteral administration, the antibody can be formulated as a solution, suspension, emulsion or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

Alternative Administration. Many known and developed modes of administration can be used according to the present invention for administering pharmaceutically effective amounts of at least one anti-TNF antibody according to the present invention. While pulmonary administration is used in the following description, other modes of administration can be used according to the present invention with suitable results.

TNF antibodies of the present invention can be delivered in a carrier, as a solution, emulsion, colloid, or suspension, or as a dry powder, using any of a variety of devices and methods suitable for administration by inhalation or other modes described here within or known in the art.

Parenteral Formulations and Administration. Formulations for parenteral administration can contain as common excipients sterile water or saline, polyalkylene glycols such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent such as aqueous solution or a sterile injectable solution or suspension in a solvent. As the usable vehicle or solvent, water, Ringer's solution, isotonic saline, etc. are allowed; as an ordinary solvent, or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

Alternative Delivery. The invention further relates to the administration of at least one anti-TNF antibody by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal means. At least one anti-TNF antibody composition can be prepared for use for parenteral (subcutaneous, intramuscular or intravenous) or any other administration particularly in the form of liquid solutions or suspensions; for use in vaginal or rectal administration particularly in semisolid forms such as, but not limited to, creams and suppositories; for buccal, or sublingual administration such as, but not limited to, in the form of tablets or capsules; or intranasally such as, but not limited to, the form of powders, nasal drops or aerosols or certain agents; or transdermally such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement”; Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways such as electroporation, or to increase the mobility of charged drugs through the skin such as iontophoresis, or application of ultrasound such as sonophoresis (U.S. Pat. Nos. 4,309,989 and 4,767,402) (the above publications and patents being entirely incorporated herein by reference).

Pulmonary/Nasal Administration. For pulmonary administration, preferably at least one anti-TNF antibody composition is delivered in a particle size effective for reaching the lower airways of the lung or sinuses. According to the invention, at least one anti-TNF antibody can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers, dry powder generators, sprayers, and the like. Other devices suitable for directing the pulmonary or nasal administration of antibodies are also known in the art. All such devices can use of formulations suitable for the administration for the dispensing of antibody in an aerosol. Such aerosols can be comprised of either solution (both aqueous and non-aqueous) or solid particles. Metered dose inhalers like the VENTOLIN® (metered dose inhaler), typically use a propellant gas and require actuation during inspiration (See, e.g., WO 94/16970, WO 98/35888). Dry powder inhalers like Turbuhaler (Astra), Rotahaler (Glaxo), DISKUS® (inhaler) (Glaxo), SPIROS® (inhaler) (Dura), devices marketed by Inhale Therapeutics, and the Spinhaler powder inhaler (Fisons), use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like AERX® (nebulizer) Aradigm, the ULTRAVENT® (nebulizer) (Mallinckrodt), and the Acorn II nebulizer (Marquest Medical Products) (U.S. Pat. No. 5,404,871 Aradigm, WO 97/22376), the above references entirely incorporated herein by reference, produce aerosols from solutions, while metered dose inhalers, dry powder inhalers, etc. generate small particle aerosols. These specific examples of commercially available inhalation devices are intended to be a representative of specific devices suitable for the practice of this invention and are not intended as limiting the scope of the invention. Preferably, a composition comprising at least one anti-TNF antibody is delivered by a dry powder inhaler or a sprayer. There are a several desirable features of an inhalation device for administering at least one antibody of the present invention. For example, delivery by the inhalation device is advantageously reliable, reproducible, and accurate. The inhalation device can optionally deliver small dry particles, e.g. less than about 10 μm, preferably about 1-5 μm, for good respirability.

Administration of TNF antibody Compositions as a Spray. A spray including TNF antibody composition protein can be produced by forcing a suspension or solution of at least one anti-TNF antibody through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of at least one anti-TNF antibody composition protein delivered by a sprayer have a particle size less than about 10 μm, preferably in the range of about 1 μm to about 5 μm, and most preferably about 2 μm to about 3 μm.

Formulations of at least one anti-TNF antibody composition protein suitable for use with a sprayer typically include antibody composition protein in an aqueous solution at a concentration of about 0.1 mg to about 100 mg of at least one anti-TNF antibody composition protein per ml of solution or mg/gm, or any range or value therein, e.g., but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/ml or mg/gm. The formulation can include agents such as an excipient, a buffer, an isotonicity agent, a preservative, a surfactant, and, preferably, zinc. The formulation can also include an excipient or agent for stabilization of the antibody composition protein, such as a buffer, a reducing agent, a bulk protein, or a carbohydrate. Bulk proteins useful in formulating antibody composition proteins include albumin, protamine, or the like. Typical carbohydrates useful in formulating antibody composition proteins include sucrose, mannitol, lactose, trehalose, glucose, or the like. The antibody composition protein formulation can also include a surfactant, which can reduce or prevent surface-induced aggregation of the antibody composition protein caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan monooleate, polysorbate 80, polysorbate 20, or the like. Additional agents known in the art for formulation of a protein such as TNF antibodies, or specified portions or variants, can also be included in the formulation.

Administration of TNF antibody compositions by a Nebulizer. Antibody composition protein can be administered by a nebulizer, such as jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of antibody composition protein through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of antibody composition protein either directly or through a coupling fluid, creating an aerosol including the antibody composition protein. Advantageously, particles of antibody composition protein delivered by a nebulizer have a particle size less than about 10 μm, preferably in the range of about 1 μm to about 5 μm, and most preferably about 2 μm to about 3 μm.

Formulations of at least one anti-TNF antibody suitable for use with a nebulizer, either jet or ultrasonic, typically include a concentration of about 0.1 mg to about 100 mg of at least one anti-TNF antibody protein per ml of solution. The formulation can include agents such as an excipient, a buffer, an isotonicity agent, a preservative, a surfactant, and, preferably, zinc. The formulation can also include an excipient or agent for stabilization of the at least one anti-TNF antibody composition protein, such as a buffer, a reducing agent, a bulk protein, or a carbohydrate. Bulk proteins useful in formulating at least one anti-TNF antibody composition proteins include albumin, protamine, or the like. Typical carbohydrates useful in formulating at least one anti-TNF antibody include sucrose, mannitol, lactose, trehalose, glucose, or the like. The at least one anti-TNF antibody formulation can also include a surfactant, which can reduce or prevent surface-induced aggregation of the at least one anti-TNF antibody caused by atomization of the solution in forming an aerosol. Various conventional surfactants can be employed, such as polyoxyethylene fatty acid esters and alcohols, and polyoxyethylene sorbital fatty acid esters. Amounts will generally range between 0.001 and 4% by weight of the formulation. Especially preferred surfactants for purposes of this invention are polyoxyethylene sorbitan mono-oleate, polysorbate 80, polysorbate 20, or the like. Additional agents known in the art for formulation of a protein such as antibody protein can also be included in the formulation.

Administration of TNF antibody compositions By A Metered Dose Inhaler. In a metered dose inhaler (MDI), a propellant, at least one anti-TNF antibody, and any excipients or other additives are contained in a canister as a mixture including a liquefied compressed gas. Actuation of the metering valve releases the mixture as an aerosol, preferably containing particles in the size range of less than about 10 μm, preferably about 1 μm to about 5 μm, and most preferably about 2 μm to about 3 μm. The desired aerosol particle size can be obtained by employing a formulation of antibody composition protein produced by various methods known to those of skill in the art, including jet-milling, spray drying, critical point condensation, or the like. Preferred metered dose inhalers include those manufactured by 3M or Glaxo and employing a hydrofluorocarbon propellant.

Formulations of at least one anti-TNF antibody for use with a metered-dose inhaler device will generally include a finely divided powder containing at least one anti-TNF antibody as a suspension in a non-aqueous medium, for example, suspended in a propellant with the aid of a surfactant. The propellant can be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluroalkane-134a), HFA-227 (hydrofluroalkane-227), or the like. Preferably the propellant is a hydrofluorocarbon. The surfactant can be chosen to stabilize the at least one anti-TNF antibody as a suspension in the propellant, to protect the active agent against chemical degradation, and the like. Suitable surfactants include sorbitan trioleate, soya lecithin, oleic acid, or the like. In some cases, solution aerosols are preferred using solvents such as ethanol. Additional agents known in the art for formulation of a protein can also be included in the formulation.

One of ordinary skill in the art will recognize that the methods of the current invention can be achieved by pulmonary administration of at least one anti-TNF antibody compositions via devices not described herein.

Oral Formulations and Administration. Formulations for oral rely on the co-administration of adjuvants (e.g., resorcinols and nonionic surfactants such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant such as magnesium stearate, paraben, preserving agent such as sorbic acid, ascorbic acid, alpha-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. Nos. 5,879,681 and 5,5,871,753 are used to deliver biologically active agents orally are known in the art.

Mucosal Formulations and Administration. For absorption through mucosal surfaces, compositions and methods of administering at least one anti-TNF antibody include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. Nos. 5,514,670). Mucous surfaces suitable for application of the emulsions of the present invention can include corneal, conjunctival, buccal, sublingual, nasal, vaginal, pulmonary, stomachic, intestinal, and rectal routes of administration. Formulations for vaginal or rectal administration, e.g. suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration excipients include sugars, calcium stearate, magnesium stearate, pregelinatined starch, and the like (U.S. Pat. Nos. 5,849,695).

Transdermal Formulations and Administration. For transdermal administration, the at least one anti-TNF antibody is encapsulated in a delivery device such as a liposome or polymeric nanoparticles, microparticle, microcapsule, or microspheres (referred to collectively as microparticles unless otherwise stated). A number of suitable devices are known, including microparticles made of synthetic polymers such as polyhydroxy acids such as polylactic acid, polyglycolic acid and copolymers thereof, polyorthoesters, polyanhydrides, and polyphosphazenes, and natural polymers such as collagen, polyamino acids, albumin and other proteins, alginate and other polysaccharides, and combinations thereof (U.S. Pat. Nos. 5,814,599).

Prolonged Administration and Formulations. It can be sometimes desirable to deliver the compounds of the present invention to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b) e.g. a zinc tannate salt. Additionally, the compounds of the present invention or, preferably, a relatively insoluble salt such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulated in a slow degrading, non-toxic, non-antigenic polymer such as a polylactic acid/polyglycolic acid polymer for example as described in U.S. Pat. No. 3,773,919. The compounds or, preferably, relatively insoluble salts such as those described above can also be formulated in cholesterol matrix silastic pellets, particularly for use in animals. Additional slow release, depot or implant formulations, e.g. gas or liquid liposomes are known in the literature (U.S. Pat. No. 5,770,222 and “Sustained and Controlled Release Drug Delivery Systems”, J. R. Robinson ed., Marcel Dekker, Inc., N.Y., 1978).

Having generally described the invention, the same will be more readily understood by reference to the following examples, which are provided by way of illustration and are not intended as limiting.

### EXAMPLE 1

### Cloning and Expression of TNF Antibody in Mammalian Cells

A typical mammalian expression vector contains at least one promoter element, which mediates the initiation of transcription of mRNA, the antibody coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRES lneo, pRetro-Off, pRetro-On, PLXSN, or pLNCX (Clonetech Labs, Palo Alto, Calif.), pcDNA3.1 (+/−), pcDNA/Zeo (+/−) or pcDNA3.1/Hygro (+/−) (Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded antibody. The DHFR (dihydrofolate reductase) marker is useful to develop cell lines that carry several hundred or even several thousand copies of the gene of interest. Another useful selection marker is the enzyme glutamine synthase (GS) (Murphy, et al., Biochem. J. 227:277-279 (1991); Bebbington, et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of antibodies.

The expression vectors pC1 and pC4 contain the strong promoter (LTR) of the Rous Sarcoma Virus (Cullen, et al., Molec. Cell. Biol. 5:438-447 (1985)) plus a fragment of the CMV-enhancer (Boshart, et al., Cell 41:521-530 (1985)). Multiple cloning sites, e.g., with the restriction enzyme cleavage sites BamHI, Xbal and Asp718, facilitate the cloning of the gene of interest. The vectors contain in addition the 3′ intron, the polyadenylation and termination signal of the rat preproinsulin gene.

Cloning and Expression in CHO Cells. The vector pC4 is used for the expression of TNF antibody. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (e.g., alpha minus MEM, Life Technologies, Gaithersburg, Md.) supplemented with the chemotherapeutic agent methotrexate. The amplification of the DHFR genes in cells resistant to methotrexate (MTX) has been well documented (see, e.g., F. W. Alt, et al., J. Biol. Chem. 253:1357-1370 (1978); J. L. Hamlin and C. Ma, Biochem. et Biophys. Acta 1097:107-143 (1990); and M. J. Page and M. A. Sydenham, Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained that contain the amplified gene integrated into one or more chromosome(s) of the host cell.

Plasmid pC4 contains for expressing the gene of interest the strong promoter of the long terminal repeat (LTR) of the Rous Sarcoma Virus (Cullen, et al., Molec. Cell. Biol. 5:438-447 (1985)) plus a fragment isolated from the enhancer of the immediate early gene of human cytomegalovirus (CMV) (Boshart, et al., Cell 41:521-530 (1985)). Downstream of the promoter are BamHI, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human beta-actin promoter, the SV40 early or late promoters or the long terminal repeats from other retroviruses, e.g., HIV and HTLVI. Clontech's Tet-Off and Tet-On gene expression systems and similar systems can be used to express the TNF in a regulated way in mammalian cells (M. Gossen, and H. Bujard, Proc. Natl. Acad. Sci. USA 89: 5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The isolated variable and constant region encoding DNA and the dephosphorylated vector are then ligated with T4 DNA ligase. E. coli HB101 or XL-1 Blue cells are then transformed, and bacteria are identified that contain the fragment inserted into plasmid pC4 using, for instance, restriction enzyme analysis.

Chinese hamster ovary (CHO) cells lacking an active DHFR gene are used for transfection. 5 μg of the expression plasmid pC4 is cotransfected with 0.5 μg of the plasmid pSV2-neo using lipofectin. The plasmid pSV2neo contains a dominant selectable marker, the neo gene from Tn5 encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mM, 2 mM, 5 mM, 10 mM, 20 mM). The same procedure is repeated until clones are obtained that grow at a concentration of 100-200 mM. Expression of the desired gene product is analyzed, for instance, by SDS-PAGE and Western blot or by reverse phase HPLC analysis.

### EXAMPLE 2

### Generation of High Affinity Human IgG Monoclonal Antibodies Reactive with Human TNF Using Transgenic Mice

Summary. Transgenic mice have been used that contain human heavy and light chain immunoglobulin genes to generate high affinity, completely human, monoclonal antibodies that can be used therapeutically to inhibit the action of TNF for the treatment of one or more TNF-mediated disease. (CBA/J×C57/BL6/J) F2 hybrid mice containing human variable and constant region antibody transgenes for both heavy and light chains are immunized with human recombinant TNF (Taylor et al., Intl. Immunol. 6:579-591 (1993); Lonberg, et al., Nature 368:856-859 (1994); Neuberger, M., Nature Biotech. 14:826 (1996); Fishwild, et al., Nature Biotechnology 14:845-851 (1996)). Several fusions yielded one or more panels of completely human TNF reactive IgG monoclonal antibodies. The completely human anti-TNF antibodies are further characterized. All are IgG1κ. Such antibodies are found to have affinity constants somewhere between 1×10⁹ and 9×10¹². The unexpectedly high affinities of these fully human monoclonal antibodies make them suitable candidates for therapeutic applications in TNF related diseases, pathologies or disorders.

Abbreviations. BSA—bovine serum albumin; CO₂—carbon dioxide; DMSO—dimethyl sulfoxide; EIA—enzyme immunoassay; FBS—fetal bovine serum; H₂O₂—hydrogen peroxide; HRP—horseradish peroxidase; ID—interadermal; Ig—immunoglobulin; TNF—tissue necrosis factor alpha; IP—intraperitoneal; IV—intravenous; Mab or mAb—monoclonal antibody; OD—optical density; OPD—o-Phenylenediamine dihydrochloride; PEG—polyethylene glycol; PSA—penicillin, streptomycin, amphotericin; RT—room temperature; SQ—subcutaneous; v/v—volume per volume; w/v—weight per volume.

### Materials and Methods

Animals. Transgenic mice that can express human antibodies are known in the art (and are commercially available (e.g., from GenPharm International, San Jose, Calif.; Abgenix, Freemont, Calif., and others) that express human immunoglobulins but not mouse IgM or Igx. For example, such transgenic mice contain human sequence transgenes that undergo V(D)J joining, heavy-chain class switching, and somatic mutation to generate a repertoire of human sequence immunoglobulins (Lonberg, et al., Nature 368:856-859 (1994)). The light chain transgene can be derived, e.g., in part from a yeast artificial chromosome clone that includes nearly half of the germline human Vκ region. In addition, the heavy-chain transgene can encode both human μ and human γ1 (Fishwild, et al., Nature Biotechnology 14:845-851 (1996)) and/or γ3 constant regions. Mice derived from appropriate genotypic lineages can be used in the immunization and fusion processes to generate fully human monoclonal antibodies to TNF.

Immunization. One or more immunization schedules can be used to generate the anti-TNF human hybridomas. The first several fusions can be performed after the following exemplary immunization protocol, but other similar known protocols can be used. Several 14-20 week old female and/or surgically castrated transgenic male mice are immunized IP and/or ID with 1-1000 μg of recombinant human TNF emulsified with an equal volume of TITERMAX or complete Freund's adjuvant in a final volume of 100-400 μL (e.g., 200). Each mouse can also optionally receive 1-10 μg in 100 μL physiological saline at each of 2 SQ sites. The mice can then be immunized 1-7, 5-12, 10-18, 17-25 and/or 21-34 days later IP (1-400 μg) and SQ (1-400 μg×2) with TNF emulsified with an equal volume of TITERMAX or incomplete Freund's adjuvant. Mice can be bled 12-25 and 25-40 days later by retro-orbital puncture without anti-coagulant. The blood is then allowed to clot at RT for one hour and the serum is collected and titered using an TNF EIA assay according to known methods. Fusions are performed when repeated injections do not cause titers to increase. At that time, the mice can be given a final IV booster injection of 1-400 μg TNF diluted in 100 μL physiological saline. Three days later, the mice can be euthanized by cervical dislocation and the spleens removed aseptically and immersed in 10 mL of cold phosphate buffered saline (PBS) containing 100 U/mL penicillin, 100 μg/mL streptomycin, and 0.25 μg/mL amphotericin B (PSA). The splenocytes are harvested by sterilely perfusing the spleen with PSA-PBS. The cells are washed once in cold PSA-PBS, counted using Trypan blue dye exclusion and resuspended in RPMI 1640 media containing 25 mM Hepes.

Cell Fusion. Fusion can be carried out at a 1:1 to 1:10 ratio of murine myeloma cells to viable spleen cells according to known methods, e.g., as known in the art. As a non-limiting example, spleen cells and myeloma cells can be pelleted together. The pellet can then be slowly resuspended, over 30 seconds, in 1 mL of 50% (w/v) PEG/PBS solution (PEG molecular weight 1,450, Sigma) at 37□ C. The fusion can then be stopped by slowly adding 10.5 mL of RPMI 1640 medium containing 25 mM Hepes (37□ C.) over 1 minute. The fused cells are centrifuged for 5 minutes at 500-1500 rpm. The cells are then resuspended in HAT medium (RPMI 1640 medium containing 25 mM Hepes, 10% Fetal Clone I serum (Hyclone), 1 mM sodium pyruvate, 4 mM L-glutamine, 10 μg/mL gentamicin, 2.5% Origen culturing supplement (Fisher), 10% 653-conditioned RPMI 1640/Hepes media, 50 μM 2-mercaptoethanol, 100 μM hypoxanthine, 0.4 μM aminopterin, and 16 μM thymidine) and then plated at 200 μL/well in fifteen 96-well flat bottom tissue culture plates. The plates are then placed in a humidified 37 □ C. incubator containing 5% CO₂ and 95% air for 7-10 days.

Detection of Human IgG Anti-TNF Antibodies in Mouse Serum. Solid phase EIA's can be used to screen mouse sera for human IgG antibodies specific for human TNF. Briefly, plates can be coated with TNF at 2 μg/mL in PBS overnight. After washing in 0.15M saline containing 0.02% (v/v) Tween 20, the wells can be blocked with 1% (w/v) BSA in PBS, 200 μL/well for 1 hour at RT. Plates are used immediately or frozen at −20□ C. for future use. Mouse serum dilutions are incubated on the TNF coated plates at 50 μL/well at RT for 1 hour. The plates are washed and then probed with 50 μL/well HRP-labeled goat anti-human IgG, Fc specific diluted 1:30,000 in 1% BSA-PBS for 1 hour at RT. The plates can again be washed and 100 μL/well of the citrate-phosphate substrate solution (0.1M citric acid and 0.2M sodium phosphate, 0.01% H₂O₂ and 1 mg/mL OPD) is added for 15 minutes at RT. Stop solution (4N sulfuric acid) is then added at 25 μL/well and the OD's are read at 490 nm via an automated plate spectrophotometer.

Detection of Completely Human Immunoglobulins in Hybridoma Supernates. Growth positive hybridomas secreting fully human immunoglobulins can be detected using a suitable EIA. Briefly, 96 well pop-out plates (VWR, 610744) can be coated with 10 μg/mL goat anti-human IgG Fc in sodium carbonate buffer overnight at 4□ C. The plates are washed and blocked with 1% BSA-PBS for one hour at 37° C. and used immediately or frozen at −20□ C. Undiluted hybridoma supernatants are incubated on the plates for one hour at 37° C. The plates are washed and probed with HRP labeled goat anti-human kappa diluted 1:10,000 in 1% BSA-PBS for one hour at 37° C. The plates are then incubated with substrate solution as described above.

Determination of Fully Human Anti-TNF Reactivity. Hybridomas, as above, can be simultaneously assayed for reactivity to TNF using a suitable RIA or other assay. For example, supernatants are incubated on goat anti-human IgG Fc plates as above, washed and then probed with radiolabled TNF with appropriate counts per well for 1 hour at RT. The wells are washed twice with PBS and bound radiolabled TNF is quantitated using a suitable counter.

Human IgG1κ anti-TNF secreting hybridomas can be expanded in cell culture and serially subcloned by limiting dilution. The resulting clonal populations can be expanded and cryopreserved in freezing medium (95% FBS, 5% DMSO) and stored in liquid nitrogen.

Isotyping. Isotype determination of the antibodies can be accomplished using an EIA in a format similar to that used to screen the mouse immune sera for specific titers. TNF can be coated on 96-well plates as described above and purified antibody at 2 μg/mL can be incubated on the plate for one hour at RT. The plate is washed and probed with HRP labeled goat anti-human IgG₁ or HRP labeled goat anti-human IgG₃ diluted at 1:4000 in 1% BSA-PBS for one hour at RT. The plate is again washed and incubated with substrate solution as described above.

Binding Kinetics of Human Anti-Human TNF Antibodies With Human TNF. Binding characteristics for antibodies can be suitably assessed using an TNF capture EIA and BIAcore technology, for example. Graded concentrations of purified human TNF antibodies can be assessed for binding to EIA plates coated with 2 μg/mL of TNF in assays as described above. The OD's can be then presented as semi-log plots showing relative binding efficiencies.

Quantitative binding constants can be obtained, e.g., as follows, or by any other known suitable method. A BIAcore CM-5 (carboxymethyl) chip is placed in a BIAcore 2000 unit. HBS buffer (0.01 M HEPES, 0.15 M NaCl, 3 mM EDTA, 0.005% v/v P20 surfactant, pH 7.4) is flowed over a flow cell of the chip at 5 μL/minute until a stable baseline is obtained. A solution (100 μL) of 15 mg of EDC (N-ethyl-N′-(3-dimethyl-aminopropyl)-carbodiimide hydrochloride) in 200 μL water is added to 100 μL of a solution of 2.3 mg of NHS (N-hydroxysuccinimide) in 200 μL water. Forty (40) μL of the resulting solution is injected onto the chip. Six μL of a solution of human TNF (15 μg/mL in 10 mM sodium acetate, pH 4.8) is injected onto the chip, resulting in an increase of ca. 500 RU. The buffer is changed to TBS/Ca/Mg/BSA running buffer (20 mM Tris, 0.15 M sodium chloride, 2 mM calcium chloride, 2 mM magnesium acetate, 0.5% Triton X-100, 25 μg/mL BSA, pH 7.4) and flowed over the chip overnight to equilibrate it and to hydrolyze or cap any unreacted succinimide esters.

Antibodies are dissolved in the running buffer at 33.33, 16.67, 8.33, and 4.17 nM.

The flow rate is adjusted to 30 μL/min and the instrument temperature to 25□ C. Two flow cells are used for the kinetic runs, one on which TNF had been immobilized (sample) and a second, underivatized flow cell (blank). 120 μL of each antibody concentration is injected over the flow cells at 30 μL/min (association phase) followed by an uninterrupted 360 seconds of buffer flow (dissociation phase). The surface of the chip is regenerated (tissue necrosis factor alpha /antibody complex dissociated) by two sequential injections of 30 μL each of 2 M guanidine thiocyanate.

Analysis of the data is done using BIA evaluation 3.0 or CLAMP 2.0, as known in the art. For each antibody concentration the blank sensogram is subtracted from the sample sensogram. A global fit is done for both dissociation (k_{d}, sec⁻¹) and association (kₐ, mol⁻¹ sec⁻¹) and the dissociation constant (K_{D}, mol) calculated (k_{d}/kₐ). Where the antibody affinity is high enough that the RUs of antibody captured are >100, additional dilutions of the antibody are run.

### Results and Discussion

Generation of Anti-Human TNF Monoclonal Antibodies. Several fusions are performed, and each fusion is seeded in 15 plates (1440 wells/fusion) that yield several dozen antibodies specific for human TNF. Of these, some are found to consist of a combination of human and mouse Ig chains. The remaining hybridomas secret anti-TNF antibodies consisting solely of human heavy and light chains. Of the human hybridomas all are expected to be IgG1κ.

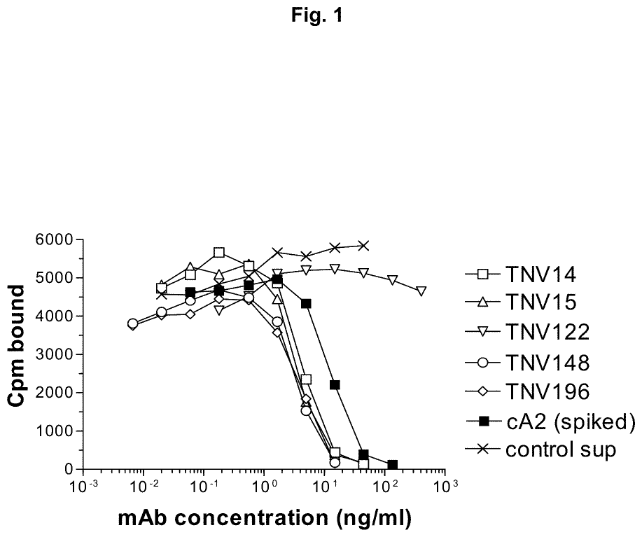

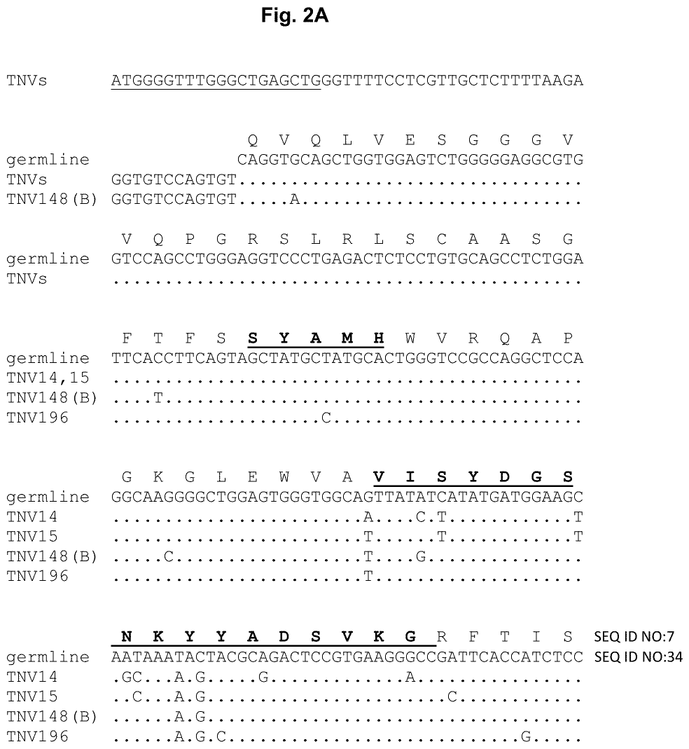

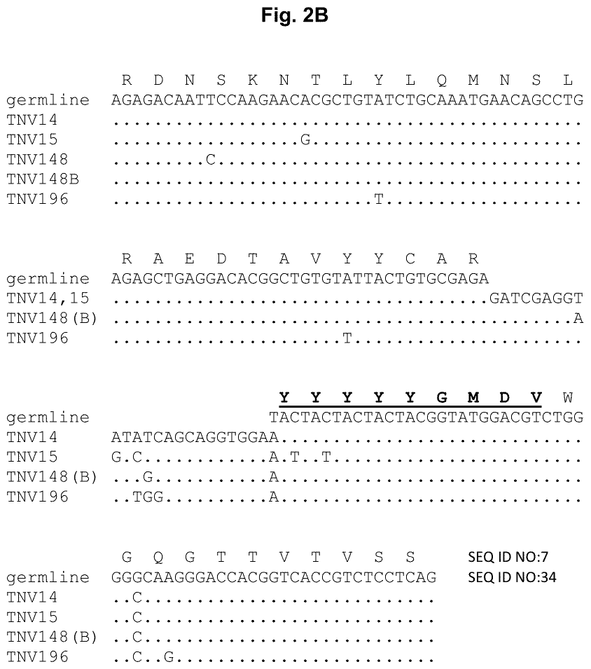

Binding Kinetics of Human Anti-Human TNF Antibodies. ELISA analysis confirms that purified antibody from most or all of these hybridomas bind TNF in a concentration-dependent manner. FIG. 1 and FIG. 2A-B show the results of the relative binding efficiency of these antibodies. In this case, the avidity of the antibody for its cognate antigen (epitope) is measured. It should be noted that binding TNF directly to the EIA plate can cause denaturation of the protein and the apparent binding affinities cannot be reflective of binding to undenatured protein. Fifty percent binding is found over a range of concentrations.

Quantitative binding constants are obtained using BlAcore analysis of the human antibodies and reveals that several of the human monoclonal antibodies are very high affinity with K_{D} in the range of 1×10⁻⁹ to 7×10⁻¹².

### Conclusions.

Several fusions are performed utilizing splenocytes from hybrid mice containing human variable and constant region antibody transgenes that are immunized with human TNF. A set of several completely human TNF reactive IgG monoclonal antibodies of the IgG1κ isotype were generated. The completely human anti-TNF antibodies are further characterized. Several of generated antibodies have affinity constants between 1×10⁹ and 9×10¹². The unexpectedly high affinities of these fully human monoclonal antibodies make them suitable for therapeutic applications in TNF-dependent diseases, pathologies or related conditions.

### EXAMPLE 3

### Generation of Human IgG Monoclonal Antibodies Reactive to Human TNFα

Summary. (CBA/J×C57BL/6J) F2 hybrid mice (1-4) containing human variable and constant region antibody transgenes for both heavy and light chains were immunized with recombinant human TNFα. One fusion, named GenTNV, yielded eight totally human IgG1κ monoclonal antibodies that bind to immobilized recombinant human TNFα. Shortly after identification, the eight cell lines were transferred to Molecular Biology for further characterization. As these Mabs are totally human in sequence, they are expected to be less immunogenic than cA2 (Remicade) in humans.

Abbreviations. BSA—bovine serum albumin; CO₂—carbon dioxide; DMSO—dimethyl sulfoxide; EIA—enzyme immunoassay; FBS—fetal bovine serum; H₂O₂—hydrogen peroxide; HC—heavy chain; HRP—horseradish peroxidase; ID—interadermal; Ig—immunoglobulin; TNF—tissue necrosis factor alpha; IP—intraperitoneal; IV—intravenous; Mab—monoclonal antibody; OD—optical density; OPD—o-Phenylenediamine dihydrochloride; PEG—polyethylene glycol; PSA—penicillin, streptomycin, amphotericin; RT—room temperature; SQ—subcutaneous; TNFα—tumor necrosis factor alpha; v/v—volume per volume; w/v—weight per volume.

Introduction. Transgenic mice that contain human heavy and light chain immunoglobulin genes were utilized to generate totally human monoclonal antibodies that are specific to recombinant human TNFα. It is hoped that these unique antibodies can be used, as cA2 (Remicade) is used to therapeutically inhibit the inflammatory processes involved in TNFα-mediated disease with the benefit of increased serum half-life and decreased side effects relating to immunogenicity.

As defined herein, the term “half-life” indicates that the plasma concentration of a drug (e.g., a therapeutic anti-TNFα antibody) is halved after one elimination half-life. Therefore, in each succeeding half-life, less drug is eliminated. After one half-life the amount of drug remaining in the body is 50% after two half-lives 25%, etc. The half-life of a drug depends on its clearance and volume of distribution. The elimination half-life is considered to be independent of the amount of drug in the body.

### Materials and Methods.

Animals. Transgenic mice that express human immunoglobulins, but not mouse IgM or Igκ, have been developed by GenPharm International. These mice contain functional human antibody transgenes that undergo V(D)J joining, heavy-chain class switching and somatic mutation to generate a repertoire of antigen-specific human immunoglobulins (1). The light chain transgenes are derived in part from a yeast artificial chromosome clone that includes nearly half of the germline human Vκ locus. In addition to several VH genes, the heavy-chain (HC) transgene encodes both human μ and human γ1 (2) and/or γ3 constant regions. A mouse derived from the HCo12/KCo5 genotypic lineage was used in the immunization and fusion process to generate the monoclonal antibodies described here.

Purification of Human TNFα. Human TNFα was purified from tissue culture supernatant from C237A cells by affinity chromatography using a column packed with the TNFα receptor-Fc fusion protein (p55-sf2) (5) coupled to Sepharose 4B (Pharmacia). The cell supernatant was mixed with one-ninth its volume of 10× Dulbecco's PBS (D-PBS) and passed through the column at 4° C. at 4 mL/min. The column was then washed with PBS and the TNFα was eluted with 0.1 M sodium citrate, pH 3.5 and neutralized with 2 M Tris-HCl pH 8.5. The purified TNFα was buffer exchanged into 10 mM Tris, 0.12 M sodium chloride pH 7.5 and filtered through a 0.2 um syringe filter.

Immunizations. A female GenPharm mouse, approximately 16 weeks old, was immunized IP (200 μL) and ID (100 μL at the base of the tail) with a total of 100 μg of TNFα (lot JG102298 or JG102098) emulsified with an equal volume of Titermax adjuvant on days 0, 12 and 28. The mouse was bled on days 21 and 35 by retro-orbital puncture without anti-coagulant. The blood was allowed to clot at RT for one hour and the serum was collected and titered using TNFα solid phase EIA assay. The fusion, named GenTNV, was performed after the mouse was allowed to rest for seven weeks following injection on day 28. The mouse, with a specific human IgG titer of 1:160 against TNFα, was then given a final IV booster injection of 50 μg TNFα diluted in 100 μL physiological saline. Three days later, the mouse was euthanized by cervical dislocation and the spleen was removed aseptically and immersed in 10 mL of cold phosphate-buffered saline (PBS) containing 100 U/mL penicillin, 100 μg/mL streptomycin, and 0.25 μg/mL amphotericin B (PSA). The splenocytes were harvested by sterilely perfusing the spleen with PSA-PBS. The cells were washed once in cold PSA-PBS, counted using a Coulter counter and resuspended in RPMI 1640 media containing 25 mM Hepes.

Cell Lines. The non-secreting mouse myeloma fusion partner, 653 was received into Cell Biology Services (CBS) group on 5-14-97 from Centocor's Product Development group. The cell line was expanded in RPMI medium (JRH Biosciences) supplemented with 10% (v/v) FBS (Cell Culture Labs), 1 mM sodium pyruvate, 0.1 mM NEAA, 2 mM L-glutamine (all from JRH Biosciences) and cryopreserved in 95% FBS and 5% DMSO (Sigma), then stored in a vapor phase liquid nitrogen freezer in CBS. The cell bank was sterile (Quality Control Centocor, Malvern) and free of mycoplasma (Bionique Laboratories). Cells were maintained in log phase culture until fusion. They were washed in PBS, counted, and viability determined (>95%) via trypan blue dye exclusion prior to fusion.

Human TNFα was produced by a recombinant cell line, named C237A, generated in Molecular Biology at Centocor. The cell line was expanded in IMDM medium (JRH Biosciences) supplemented with 5% (v/v) FBS (Cell Culture Labs), 2 mM L-glutamine (all from JRH Biosciences), and 0.5 :g/mL mycophenolic acid, and cryopreserved in 95% FBS and 5% DMSO (Sigma), then stored in a vapor phase liquid nitrogen freezer in CBS (13). The cell bank was sterile (Quality Control Centocor, Malvern) and free of mycoplasma (Bionique Laboratories).

Cell Fusion. The cell fusion was carried out using a 1:1 ratio of 653 murine myeloma cells and viable murine spleen cells. Briefly, spleen cells and myeloma cells were pelleted together. The pellet was slowly resuspended over a 30 second period in 1 mL of 50% (w/v) PEG/PBS solution (PEG molecular weight of 1,450 g/mole, Sigma) at 37° C. The fusion was stopped by slowly adding 10.5 mL of RPMI media (no additives) (JRH) (37° C.) over 1 minute. The fused cells were centrifuged for 5 minutes at 750 rpm. The cells were then resuspended in HAT medium (RPMI/HEPES medium containing 10% Fetal Bovine Serum (JRH), 1 mM sodium pyruvate, 2 mM L-glutamine, 10 μg/mL gentamicin, 2.5% Origen culturing supplement (Fisher), 50 μM 2-mercaptoethanol, 1% 653-conditioned RPMI media, 100 μM hypoxanthine, 0.4 μM aminopterin, and 16 μM thymidine) and then plated at 200 μL/well in five 96-well flat bottom tissue culture plates. The plates were then placed in a humidified 37° C. incubator containing 5% CO₂ and 95% air for 7-10 days.

Detection of Human IgG Anti-TNFα Antibodies in Mouse Serum. Solid phase EIAs were used to screen mouse sera for human IgG antibodies specific for human TNFα. Briefly, plates were coated with TNFα at 1 μg/mL in PBS overnight. After washing in 0.15 M saline containing 0.02% (v/v) Tween 20, the wells were blocked with 1% (w/v) BSA in PBS, 200 μL/well for 1 hour at RT. Plates were either used immediately or frozen at −20° C. for future use. Mouse sera were incubated in two-fold serial dilutions on the human TNFα-coated plates at 50 μL/well at RT for 1 hour. The plates were washed and then probed with 50 μL/well HRP-labeled goat anti-human IgG, Fc specific (Accurate) diluted 1:30,000 in 1% BSA-PBS for 1 hour at RT. The plates were again washed and 100 μL/well of the citrate-phosphate substrate solution (0.1 M citric acid and 0.2 M sodium phosphate, 0.01% H₂O₂ and 1 mg/mL OPD) was added for 15 minutes at RT. Stop solution (4N sulfuric acid) was then added at 25 μL/well and the OD's were read at 490 nm using an automated plate spectrophotometer.

Detection of Totally Human Immunoglobulins in Hybridoma Supernatants. Because the GenPharm mouse is capable of generating both mouse and human immunoglobulin chains, two separate EIA assays were used to test growth-positive hybridoma clones for the presence of both human light chains and human heavy chains. Plates were coated as described above and undiluted hybridoma supernatants were incubated on the plates for one hour at 37° C. The plates were washed and probed with either HRP-conjugated goat anti-human kappa (Southern Biotech) antibody diluted 1:10,000 in 1% BSA-HBSS or HRP-conjugated goat anti-human IgG Fc specific antibody diluted to 1:30,000 in 1% BSA-HBSS for one hour at 37° C. The plates were then incubated with substrate solution as described above. Hybridoma clones that did not give a positive signal in both the anti-human kappa and anti-human IgG Fc EIA formats were discarded.

Isotyping. Isotype determination of the antibodies was accomplished using an EIA in a format similar to that used to screen the mouse immune sera for specific titers. EIA plates were coated with goat anti-human IgG (H+L) at 10 :g/mL in sodium carbonate buffer overnight at 4EC and blocked as described above. Neat supernatants from 24 well cultures were incubated on the plate for one hour at RT. The plate was washed and probed with HRP-labeled goat anti-human IgG₁, IgG₂, IgG₃ or IgG₄ (Binding Site) diluted at 1:4000 in 1% BSA-PBS for one hour at RT. The plate was again washed and incubated with substrate solution as described above.

Results and Discussion. Generation of Totally Human Anti-Human TNFα Monoclonal Antibodies. One fusion, named GenTNV, was performed from a GenPharm mouse immunized with recombinant human TNFα protein. From this fusion, 196 growth-positive hybrids were screened. Eight hybridoma cell lines were identified that secreted totally human IgG antibodies reactive with human TNFα. These eight cell lines each secreted immunoglobulins of the human IgG1κ isotype and all were subcloned twice by limiting dilution to obtain stable cell lines (>90% homogeneous). Cell line names and respective C code designations are listed in Table 1. Each of the cell lines was frozen in 12-vial research cell banks stored in liquid nitrogen.

Parental cells collected from wells of a 24-well culture dish for each of the eight cell lines were handed over to Molecular Biology group on 2-18-99 for transfection and further characterization.

TABLE 1
GenTNV Cell Line Designations

| | C Code |
|---|---|
| Name | Designation |
| GenTNV14.17.12 | C414A |
| GenTNV15.28.11 | C415A |
| GenTNV32.2.16 | C416A |
| GenTNV86.14.34 | C417A |
| GenTNV118.3.36 | C418A |
| GenTNV122.23.2 | C419A |
| GenTNV148.26.12 | C420A |
| GenTNV196.9.1 | C421A |

### Conclusion.

The GenTNV fusion was performed utilizing splenocytes from a hybrid mouse containing human variable and constant region antibody transgenes that was immunized with recombinant human TNFα prepared at Centocor. Eight totally human, TNFα-reactive IgG monoclonal antibodies of the IgG1κ isotype were generated. Parental cell lines were transferred to Molecular Biology group for further characterization and development. One of these new human antibodies may prove useful in anti-inflammatory with the potential benefit of decreased immunogenicity and allergic-type complications as compared with Remicade.

References:

Taylor, et al., International Immunology 6:579-591 (1993).

Lonberg, et al., Nature 368:856-859 (1994).

Neuberger, M. Nature Biotechnology 14:826 (1996).

Fishwild, et al., Nature Biotechnology 14:845-851 (1996).

Scallon, et al., Cytokine 7:759-770 (1995).

### EXAMPLE 4

### Cloning and Preparation of Cell Lines Expressing Human Anti-TNFα Antibody

Summary. A panel of eight human monoclonal antibodies (mAbs) with a TNV designation were found to bind immobilized human TNFα with apparently high avidity. Seven of the eight mAbs were shown to efficiently block huTNFα binding to a recombinant TNF receptor. Sequence analysis of the DNA encoding the seven mAbs confirmed that all the mAbs had human V regions. The DNA sequences also revealed that three pairs of the mAbs were identical to each other, such that the original panel of eight mAbs contained only four distinct mAbs, represented by TNV14, TNV15, TNV148, and TNV196. Based on analyses of the deduced amino acid sequences of the mAbs and results of in vitro TNFα neutralization data, mAb TNV148 and TNV14 were selected for further study.

Because the proline residue at position 75 (framework 3) in the TNV148 heavy chain was not found at that position in other human antibodies of the same subgroup during a database search, site-directed DNA mutagenesis was performed to encode a serine residue at that position in order to have it conform to known germline framework e sequences. The serine modified mAb was designated TNV148B. PCR-amplified DNA encoding the heavy and light chain variable regions of TNV148B and TNV14 was cloned into newly prepared expression vectors that were based on the recently cloned heavy and light chain genes of another human mAb (12B75), disclosed in U.S. patent application No. 60/236,827, filed Oct. 7, 2000, entitled IL-12 Antibodies, Compositions, Methods and Uses, published as WO 02/12500which is entirely incorporated herein by reference.

P3X63Ag8.653 (653) cells or Sp2/0-Ag14 (Sp2/0) mouse myeloma cells were transfected with the respective heavy and light chain expression plasmids and screened through two rounds of subcloning for cell lines producing high levels of recombinant TNV148B and TNV14 (rTNV148B and rTNV14) mAbs. Evaluations of growth curves and stability of mAb production over time indicated that 653-transfectant clones C466D and C466C stably produced approximately 125 :g/ml of rTNV148B mAb in spent cultures whereas Sp2/0 transfectant 1.73-12-122 (C467A) stably produced approximately 25 :g/ml of rTNV148B mAb in spent cultures. Similar analyses indicated that Sp2/0-transfectant clone C476A produced 18 :g/ml of rTNV14 in spent cultures.

Introduction. A panel of eight mAbs derived from human TNFα-immunized GenPharm/Medarex mice (HCo12/KCo5 genotype) were previously shown to bind human TNFα and to have a totally human IgG1, kappa isotype. A simple binding assay was used to determine whether the exemplary mAbs of the invention were likely to have TNFα-neutralizing activity by evaluating their ability to block TNFα from binding to recombinant TNF receptor. Based on those results, DNA sequence results, and in vitro characterizations of several of the mAbs, TNV148 was selected as the mAb to be further characterized.

DNA sequences encoding the TNV148 mAb were cloned, modified to fit into gene expression vectors that encode suitable constant regions, introduced into the well-characterized 653 and Sp2/0 mouse myeloma cells, and resulting transfected cell lines screened until subclones were identified that produced 40-fold more mAb than the original hybridoma cell line.

### Materials and Methods.

Reagents and Cells. TRIZOL reagent was purchased from Gibco BRL. Proteinase K was obtained from Sigma Chemical Company. Reverse Transcriptase was obtained from Life Sciences, Inc. Taq DNA Polymerase was obtained from either Perkin Elmer Cetus or Gibco BRL. Restriction enzymes were purchased from New England Biolabs. QIAquick PCR Purification Kit was from Qiagen. A QuikChange Site-Directed Mutagenesis Kit was purchased from Stratagene. Wizard plasmid miniprep kits and RNasin were from Promega. Optiplates were obtained from Packard. ¹²⁵Iodine was purchased from Amersham. Custom oligonucleotides were purchased from Keystone/Biosource International. The names, identification numbers, and sequences of the oligonucleotides used in this work are shown in Table 2.

The amino acids encoded by oligonucleotide 5′14s and HuH-J6 are shown above the sequence. The ‘M’ amino acid residue represents the translation start codon. The underlined sequences in oligonucleotides 5′14s and HuH-J6 mark the BsiWI and BstBI restriction sites, respectively. The slash in HuH-J6 corresponds to the exon/intron boundary. Note that oligonucleotides whose sequence corresponds to the minus strand are written in a 3′-5′ orientation.

TABLE 2
Oligonucleotides used to clone, engineer, or sequence the TNV
mAb genes.

| Name | ID. | Sequence |
|---|---|---|
| HG1-4b | 119 | 3′-TTGGTCCAGTCGGACTGG-5′ |
| | | (SEQ ID NO: 10) |
| HG1-5b | 354 | 3′-CACCTGCACTCGGTGCTT-5′ |
| | | (SEQ ID NO: 11) |
| HG1hg | 360 | 3′-CACTGTTTTGAGTGTGTACGGGCTTAAGTT-5′ |
| | | (SEQ ID NO: 12) |
| HG1-6 | 35 | 3′-GCCGCACGTGTGGAAGGG-5′ |
| | | (SEQ ID NO: 13) |
| HCK1-3E | 117 | 3′-AGTCAAGGTCGGACTGGCTTAAGTT-5′ |
| | | (SEQ ID NO: 14) |
| HuK-3′Hd | 208 | 3′-GTTGTCCCCTCTCACAATCTTCGAATTT-5′ |
| | | (SEQ ID NO: 15) |
| HVKRNAseq | 34 | 3′-GGCGGTAGACTACTCGTC-5′ |
| | | (SEQ ID NO: 16) |
| BsiWI | | M D W T W S I |
| | | (SEQ ID NO: 17) |
| 5′14s | 366 | 5-TTTCGTACGCCACCATGGACTGGACCTGGAGCATC-3′ |
| | | (SEQ ID NO: 18) |
| 5′46s | 367 | 5′-TTTCGTACGCCACCATGGGGTTTGGGCTGAGCTG-3′ |
| | | (SEQ ID NO: 19) |
| 5′47s | 368 | 5′-TTTCGTACGCCACCATGGAGTTTGGGCTGAGCATG-3′ |
| | | (SEQ ID NO: 20) |
| 5′63s | 369 | 5′-TTTCGTACGCCACCATGAAACACCTGTGGTTCTTC-3′ |
| | | (SEQ ID NO: 21) |
| 5′73s | 370 | 5′-TTTCGTACGCCACCATGGGGTCAACCGCCATCCTC-3′ |
| | | (SEQ ID NO: 22) |
| BstBI | | T V T V S |
| | | (SEQ ID NO: 23) |
| HuH-J6 | 388 | 3′GTGCCAGTGGCAGAGGAGTCCATTCAAGCTTAAGTT-5′ |
| | | (SEQ ID NO: 24) |
| SalI | | M D M R V |
| | | (SEQ ID NO: 25) |
| LK7s | 362 | 5′-TTTGTCGACACCATGGACATGAGGGTCC(TC)C-3′ |
| | | (SEQ ID NO: 26) |
| LVgs | 363 | 5′-TTTGTCGACACCATGGAAGCCCCAGCTC-3′ |
| | | (SEQ ID NO: 27) |
| Afl2 | | T K V D I K |
| | | (SEQ ID NO: 28) |
| HuL-J3 | 380 | 3′CTGGTTTCACCTATAGTTTG/CATTCAGAATTCGGCGCCTTT |
| | | (SEQ ID NO: 29) |
| V148-QC1 | 399 | 5′-CATCTCCAGAGACAATtCCAAGAACACGCTGTATC-3′ |
| | | (SEQ ID NO: 30) |
| V148-QC2 | 400 | 3′-GTAGAGGTCTCTGTTAaGGTTCTTGTGCGACATAG-5′ |
| | | (SEQ ID NO: 31) |

A single frozen vial of 653 mouse myeloma cells was obtained. The vial was thawed that day and expanded in T flasks in IMDM, 5% FBS, 2 mM glutamine (media). These cells were maintained in continuous culture until they were transfected 2 to 3 weeks later with the anti-TNF DNA described here. Some of the cultures were harvested 5 days after the thaw date, pelleted by centrifugation, and resuspended in 95% FBS, 5% DMSO, aliquoted into 30 vials, frozen, and stored for future use. Similarly, a single frozen vial of Sp2/0 mouse myeloma cells was obtained. The vial was thawed, a new freeze-down prepared as described above, and the frozen vials stored in CBC freezer boxes AA and AB. These cells were thawed and used for all Sp2/0 transfections described here.

Assay for Inhibition of TNF Binding to Receptor. Hybridoma cell supernatants containing the TNV mAbs were used to assay for the ability of the mAbs to block binding of ¹²⁵I-labeled TNFα to the recombinant TNF receptor fusion protein, p55-sf2 (Scallon et al. (1995) Cytokine 7:759-770). 50:1 of p55-sf2 at 0.5 :g/ml in PBS was added to Optiplates to coat the wells during a one-hour incubation at 37° C. Serial dilutions of the eight TNV cell supernatants were prepared in 96-well round-bottom plates using PBS/0.1% BSA as diluent. Cell supernatant containing anti-IL-18 mAb was included as a negative control and the same anti-IL-18 supernatant spiked with cA2 (anti-TNF chimeric antibody, Remicade, U.S. Pat. No. 5,770,198, entirely incorporated herein by reference) was included as a positive control. ¹²⁵I-labeled TNFα (58 :Ci/:g, D. Shealy) was added to 100 :l of cell supernatants to have a final TNFα concentration of 5 ng/ml. The mixture was preincubated for one hour at RT. The coated Optiplates were washed to remove unbound p55-sf2 and 50 :l of the ¹²⁵I-TNFα/cell supernatant mixture was transferred to the Optiplates. After 2 hrs at RT, Optiplates were washed three times with PBS-Tween. 100 :l of Microscint-20 was added and the cpm bound determined using the TopCount gamma counter.

Amplification of V Genes and DNA Sequence Analysis. Hybridoma cells were washed once in PBS before addition of TRIZOL reagent for RNA preparation. Between 7×10⁶ and 1.7×10⁷ cells were resuspended in 1 ml TRIZOL. Tubes were shaken vigorously after addition of 200 μl of chloroform. Samples were centrifuged at 4° C. for 10 minutes. The aqueous phase was transferred to a fresh microfuge tube and an equal volume of isopropanol was added. Tubes were shaken vigorously and allowed to incubate at room temperature for 10 minutes. Samples were then centrifuged at 4° C. for 10 minutes. The pellets were washed once with 1 ml of 70% ethanol and dried briefly in a vacuum dryer. The RNA pellets were resuspended with 40 μl of DEPC-treated water. The quality of the RNA preparations was determined by fractionating 0.5 μl in a 1% agarose gel. The RNA was stored in a −80° C. freezer until used.

To prepare heavy and light chain cDNAs, mixtures were prepared that included 3 μl of RNA and 1 μg of either oligonucleotide 119 (heavy chain) or oligonucleotide 117 (light chain) (see Table 1) in a volume of 11.5 μl. The mixture was incubated at 70° C. for 10 minutes in a water bath and then chilled on ice for 10 minutes. A separate mixture was prepared that was made up of 2.5 μl of 10× reverse transcriptase buffer, 10 μl of 2.5 mM dNTPs, 1 μl of reverse transcriptase (20 units), and 0.4 μl of ribonuclease inhibitor RNasin (1 unit). 13.5 μl of this mixture was added to the 11.5 μl of the chilled RNA/oligonucleotide mixture and the reaction incubated for 40 minutes at 42° C. The cDNA synthesis reaction was then stored in a −20° C. freezer until used.

The unpurified heavy and light chain cDNAs were used as templates to PCR-amplify the variable region coding sequences. Five oligonucleotide pairs (366/354, 367/354, 368/354, 369/354, and 370/354, Table 1) were simultaneously tested for their ability to prime amplification of the heavy chain DNA. Two oligonucleotide pairs (362/208 and 363/208) were simultaneously tested for their ability to prime amplification of the light chain DNA. PCR reactions were carried out using 2 units of PLATINUM™ high fidelity (HIFI) Taq DNA polymerase in a total volume of 50 μl. Each reaction included 2 μl of a cDNA reaction, 10 pmoles of each oligonucleotide, 0.2 mM dNTPs, 5 μl of 10× HIFI Buffer, and 2 mM magnesium sulfate. The thermal cycler program was 95° C. for 5 minutes followed by 30 cycles of (94° C. for 30 seconds, 62° C. for 30 seconds, 68° C. for 1.5 minutes). There was then a final incubation at 68° C. for 10 minutes.

To prepare the PCR products for direct DNA sequencing, they were purified using the QIAquick™ PCR Purification Kit according to the manufacturer's protocol. The DNA was eluted from the spin column using 50 μl of sterile water and then dried down to a volume of 10 μl using a vacuum dryer. DNA sequencing reactions were then set up with 1 μl of purified PCR product, 10 μM oligonucleotide primer, 4 μl BigDye Terminator™ ready reaction mix, and 14 μl sterile water for a total volume of 20 μl. Heavy chain PCR products made with oligonucleotide pair 367/354 were sequenced with oligonucleotide primers 159 and 360. Light chain PCR products made with oligonucleotide pair 363/208 were sequenced with oligonucleotides 34 and 163. The thermal cycler program for sequencing was 25 cycles of (96° C. for 30 seconds, 50° C. for 15 seconds, 60° C. for 4 minutes) followed by overnight at 4° C. The reaction products were fractionated through a polyacrylamide gel and detected using an ABI 377 DNA Sequencer.

Site-directed Mutagenesis to Change an Amino Acid. A single nucleotide in the TNV148 heavy chain variable region DNA sequence was changed in order to replace Pro⁷⁵ with a Serine residue in the TNV148 mAb. Complimentary oligonucleotides, 399 and 400 (Table 1), were designed and ordered to make this change using the QuikChange™ site-directed mutagenesis method as described by the manufacturer. The two oligonucleotides were first fractionated through a 15% polyacrylamide gel and the major bands purified. Mutagenesis reactions were prepared using either 10 ng or 50 ng of TNV148 heavy chain plasmid template (p1753), 5 μl of 10× reaction buffer, 1 μl of dNTP mix, 125 ng of primer 399, 125 ng of primer 400, and 1 μl of Pfu DNA Polymerase. Sterile water was added to bring the total volume to 50 μl. The reaction mix was then incubated in a thermal cycler programmed to incubate at 95° C. for 30 seconds, and then cycle 14 times with sequential incubations of 95° C. for 30 seconds, 55° C. for 1 minute, 64° C. for 1 minute, and 68° C. for 7 minutes, followed by 30° C. for 2 minutes (1 cycle). These reactions were designed to incorporate the mutagenic oligonucleotides into otherwise identical, newly synthesized plasmids. To rid of the original TNV148 plasmids, samples were incubated at 37° C. for 1 hour after addition of 1 μl of DpnI endonuclease, which cleaves only the original methylated plasmid. One μl of the reaction was then used to transform Epicurian Coli XL1-Blue supercompetent E. coli by standard heat-shock methods and transformed bacteria identified after plating on LB-ampicillin agar plates. Plasmid minipreps were prepared using the Wizard™ kits as described by the manufacturer. After elution of sample from the Wizard™ column, plasmid DNA was precipitated with ethanol to further purify the plasmid DNA and then resuspended in 20 μl of sterile water. DNA sequence analysis was then performed to identify plasmid clones that had the desired base change and to confirm that no other base changes were inadvertently introduced into the TNV148 coding sequence. One μl of plasmid was subjected to a cycle sequencing reaction prepared with 3 μl of BigDye mix, 1 μl of pUC19 Forward primer, and 10 μl of sterile water using the same parameters described in Section 4.3.

Construction of Expression Vectors from 12B75 Genes. Several recombinant DNA steps were performed to prepare a new human IgG1 expression vector and a new human kappa expression vector from the previously-cloned genomic copies of the 12B75-encoding heavy and light chain genes, respectively, disclosed in U.S. patent application No. 60/236,827, filed Oct. 7, 2000, entitled IL-12 Antibodies, Compositions, Methods and Uses,_published as WO 02/12500, which is entirely incorporated herein by reference. The final vectors were designed to permit simple, one-step replacement of the existing variable region sequences with any appropriately-designed, PCR-amplified, variable region.

To modify the 12B75 heavy chain gene in plasmid p1560, a 6.85 kb BamHI/HindIII fragment containing the promoter and variable region was transferred from p1560 to pUC19 to make p1743. The smaller size of this plasmid compared to p1560 enabled use of QuikChange™ mutagenesis (using oligonucleotides BsiWI-1 and BsiWI-2) to introduce a unique BsiWI cloning site just upstream of the translation initiation site, following the manufacturer's protocol. The resulting plasmid was termed p1747. To introduce a BstBI site at the 3′ end of the variable region, a 5′ oligonucleotide primer was designed with SalI and BstBI sites. This primer was used with the pUC reverse primer to amplify a 2.75 kb fragment from p1747. This fragment was then cloned back into the naturally-occurring SalI site in the 12B75 variable region and a HindIII site, thereby introducing the unique BstB1 site. The resulting intermediate vector, designated p1750, could accept variable region fragments with BsiWI and BstBI ends. To prepare a version of heavy chain vector in which the constant region also derived from the 12B75 gene, the BamHI-HindIII insert in p1750 was transferred to pBR322 in order to have an EcoRI site downstream of the HindIII site. The resulting plasmid, p1768, was then digested with HindIII and EcoRI and ligated to a 5.7 kb HindIII-EcoRI fragment from p1744, a subclone derived by cloning the large BamHI-BamHI fragment from p1560 into pBC. The resulting plasmid, p1784, was then used as vector for the TNV Ab cDNA fragments with BsiWI and BstBI ends. Additional work was done to prepare expression vectors, p1788 and p1798, which include the IgG1 constant region from the 12B75 gene and differ from each other by how much of the 12B75 heavy chain J-C intron they contain.

To modify the 12B75 light chain gene in plasmid p1558, a 5.7 kb SalI/AflII fragment containing the 12B75 promoter and variable region was transferred from p1558 into the XhoI/AfIII sites of plasmid L28. This new plasmid, p1745, provided a smaller template for the mutagenesis step. Oligonucleotides (C340salI and C340sal2) were used to introduce a unique SalI restriction site at the 5′ end of the variable region by QuikChange™ mutagenesis. The resulting intermediate vector, p1746, had unique SalI and AflII restriction sites into which variable region fragments could be cloned. Any variable region fragment cloned into p1746 would preferably be joined with the 3′ half of the light chain gene. To prepare a restriction fragment from the 3′ half of the 12B75 light chain gene that could be used for this purpose, oligonucleotides BAHN-1 and BAHN-2 were annealed to each other to form a double-stranded linker containing the restriction sites BsiW1, AflII, HindII, and NotI and which contained ends that could be ligated into KpnI and SacI sites. This linker was cloned between the KpnI and SacI sites of pBC to give plasmid p1757. A 7.1 kb fragment containing the 12B75 light chain constant region, generated by digesting p1558 with AflII, then partially digesting with HindIII, was cloned between the AflII and HindII sites of p1757 to yield p1762. This new plasmid contained unique sites for BsiWI and AflII into which the BsiWI/AflII fragment containing the promoter and variable regions could be transferred uniting the two halves of the gene.

cDNA Cloning and Assembly of Expression Plasmids. All RT-PCR reactions (see above) were treated with Klenow enzyme to further fill in the DNA ends. Heavy chain PCR fragments were digested with restriction enzymes BsiWI and BstBI and then cloned between the BsiWI and BstBI sites of plasmid L28 (L28 used because the 12B75-based intermediate vector p1750 had not been prepared yet). DNA sequence analysis of the cloned inserts showed that the resulting constructs were correct and that there were no errors introduced during PCR amplifications. The assigned identification numbers for these L28 plasmid constructs (for TNV14, TNV15, TNV148, TNV148B, and TNV196) are shown in Table 3.

The BsiWI/BstBI inserts for TNV14, TNV148, and TNV148B heavy chains were transferred from the L28 vector to the newly prepared intermediate vector, p1750. The assigned identification numbers for these intermediate plasmids are shown in Table 2. This cloning step and subsequent steps were not done for TNV15 and TNV196. The variable regions were then transferred into two different human IgG1 expression vectors. Restriction enzymes EcoRI and HindIII were used to transfer the variable regions into Centocor's previously-used IgG1 vector, p104. The resulting expression plasmids, which encode an IgG1 of the Gm(f+) allotype, were designated p1781 (TNV14), p1782 (TNV148), and p1783 (TNV148B) (see Table 2). The variable regions were also cloned upstream of the IgG1 constant region derived from the 12B75 (GenPharm) gene. Those expression plasmids, which encode an IgG1 of the G1m(z) allotype, are also listed in Table 3.

The L28 vector or pBC vector represents the initial Ab cDNA clone. The inserts in those plasmids were transferred to an incomplete 12B75-based vector to make the intermediate plasmids. One additional transfer step resulted in the final expression plasmids that were either introduced into cells after being linearized or used to purify the mAb gene inserts prior to cell transfection. (ND)=not done.

TABLE 3
Plasmid identification numbers for various
heavy and light chain plasmids.

| | Gm(f+) | | G1m(z) | |
|---|---|---|---|---|
| | 128 vector | Intermediate | Expression | Expression |
| Mab | Plasmid ID | Plasmid ID | Plasmid ID | Plasmid ID |

Heavy Chains

| | | | | |
|---|---|---|---|---|
| TNV14 | p1751 | p1777 | p1781 | p1786 |
| TNV15 | p1752 | (ND) | (ND) | (ND) |
| TNV148 | p1753 | p1778 | p1782 | p1787 |
| TNV148B | p1760 | p1779 | p1783 | p1788 |
| TNV196 | p1754 | (ND) | (ND) | (ND) |

| | | |
|---|---|---|
| pBC vector | Intermediate | Expression |
| Plasmid ID | Plasmid ID | Plasmid ID |

Light Chains

| | | | |
|---|---|---|---|
| TNV14 | p1748 | p1755 | p1775 |
| TNV15 | p1748 | p1755 | p1775 |
| TNV148 | p1749 | p1756 | p1776 |
| TNV196 | p1749 | p1756 | p1776 |

Light chain PCR products were digested with restriction enzymes SalI and SacII and then cloned between the SalI and SacII sites of plasmid pBC. The two different light chain versions, which differed by one amino acid, were designated p1748 and p1749 (Table 2). DNA sequence analysis confirmed that these constructs had the correct sequences. The SalI/AflII fragments in p1748 and p1749 were then cloned between the SalI and AflII sites of intermediate vector p1746 to make p1755 and p1756, respectively. These 5′ halves of the light chain genes were then joined to the 3′ halves of the gene by transferring the BsiWI/AflII fragments from p1755 and p1756 to the newly prepared construct p1762 to make the final expression plasmids p1775 and p1776, respectively (Table 2).

Cell Transfections, Screening, and Subcloning. A total of 15 transfections of mouse myeloma cells were performed with the various TNV expression plasmids (see Table 3). These transfections were distinguished by whether (1) the host cells were Sp2/0 or 653; (2) the heavy chain constant region was encoded by Centocor's previous IgG1 vector or the 12B75 heavy chain constant region; (3) the mAb was TNV148B, TNV148, TNV14, or a new HC/LC combination; (4) whether the DNA was linearized plasmid or purified Ab gene insert; and (5) the presence or absence of the complete J-C intron sequence in the heavy chain gene. In addition, several of the transfections were repeated to increase the likelihood that a large number of clones could be screened.

Sp2/0 cells and 653 cells were each transfected with a mixture of heavy and light chain DNA (8-12 :g each) by electroporation under standard conditions as previously described (Knight DM et al. (1993) Molecular Immunology 30:1443-1453). For transfection numbers 1, 2, 3, and 16, the appropriate expression plasmids were linearized by digestion with a restriction enzyme prior to transfection. For example, SalI and NotI restriction enzymes were used to linearize TNV148B heavy chain plasmid p1783 and light chain plasmid p1776, respectively. For the remaining transfections, DNA inserts that contained only the mAb gene were separated from the plasmid vector by digesting heavy chain plasmids with BamHI and light chain plasmids with BsiWI and NotI. The mAb gene inserts were then purified by agarose gel electrophoresis and Qiex purification resins. Cells transfected with purified gene inserts were simultaneously transfected with 3-5 :g of Pstl-linearized pSV2gpt plasmid (p13) as a source of selectable marker. Following electroporation, cells were seeded in 96-well tissue culture dishes in IMDM, 15% FBS, 2 mM glutamine and incubated at 37° C. in a 5% CO₂ incubator. Two days later, an equal volume of IMDM, 5% FBS, 2mM glutamine, 2×MHX selection (1×MHX=0.5 :g/ml mycophenolic acid, 2.5 :g/ml hypoxanthine, 50 :g/ml xanthine) was added and the plates incubated for an additional 2 to 3 weeks while colonies formed.

Cell supernatants collected from wells with colonies were assayed for human IgG by ELISA as described. In brief, varying dilutions of the cell supernatants were incubated in 96-well EIA plates coated with polyclonal goat anti-human IgG Fc fragment and then bound human IgG was detected using Alkaline Phosphatase-conjugated goat anti-human IgG(H+L) and the appropriate color substrates. Standard curves, which used as standard the same purified mAb that was being measured in the cell supernatants, were included on each EIA plate to enable quantitation of the human IgG in the supernatants. Cells in those colonies that appeared to be producing the most human IgG were passaged into 24-well plates for additional production determinations in spent cultures and the highest-producing parental clones were subsequently identified.

The highest-producing parental clones were subcloned to identify higher-producing subclones and to prepare a more homogenous cell line. 96-well tissue culture plates were seeded with one cell per well or four cells per well in of IMDM, 5% FBS, 2 mM glutamine, 1×MHX and incubated at 37° C. in a 5% CO₂ incubator for 12 to 20 days until colonies were apparent. Cell supernatants were collected from wells that contained one colony per well and analyzed by ELISA as described above. Selected colonies were passaged to 24-well plates and the cultures allowed to go spent before identifying the highest-producing subclones by quantitating the human IgG levels in their supernatants. This process was repeated when selected first-round subclones were subjected to a second round of subcloning. The best second-round subclones were selected as the cell lines for development.

Characterization of Cell Subclones. The best second-round subclones were chosen and growth curves performed to evaluate mAb production levels and cell growth characteristics. T75 flasks were seeded with 1×10⁵ cells/ml in 30 ml IMDM, 5% FBS, 2 mM glutamine, and 1×MHX (or serum-free media). Aliquots of 300 μl were taken at 24 hr intervals and live cell density determined. The analyses continued until the number of live cells was less than 1×10⁵ cells/ml. The collected aliquots of cell supernatants were assayed for the concentration of antibody present. ELISA assays were performed using as standard rTNV148B or rTNV14 JG92399. Samples were incubated for 1 hour on ELISA plates coated with polyclonal goat anti-human IgG Fc and bound mAb detected with Alkaline Phosphatase-conjugated goat anti-human IgG(H+L) at a 1:1000 dilution.

A different growth curve analysis was also done for two cell lines for the purpose of comparing growth rates in the presence of varying amounts of MHX selection. Cell lines C466A and C466B were thawed into MHX-free media (IMDM, 5% FBS, 2 mM glutamine) and cultured for two additional days. Both cell cultures were then divided into three cultures that contained either no MHX, 0.2×MHX, or 1×MHX (1×MHX=0.5 :g/ml mycophenolic acid, 2.5 :g/ml hypoxanthine, 50 :g/ml xanthine). One day later, fresh T75 flasks were seeded with the cultures at a starting density of 1×10⁵ cells/ml and cells counted at 24 hour intervals for one week. Aliquots for mAb production were not collected. Doubling times were calculated for these samples using the formula provided in SOP PD32.025.

Additional studies were performed to evaluate stability of mAb production over time. Cultures were grown in 24-well plates in IMDM, 5% FBS, 2 mM glutamine, either with or without MHX selection. Cultures were split into fresh cultures whenever they became confluent and the older culture was then allowed to go spent. At this time, an aliquot of supernatant was taken and stored at 4° C. Aliquots were taken over a 55-78 day period. At the end of this period, supernatants were tested for amount of antibody present by the anti-human IgG Fc ELISA as outlined above.

### Results and Discussion.

### Inhibition of TNF Binding to Recombinant Receptor.

A simple binding assay was done to determine whether the eight TNV mAbs contained in hybridoma cell supernatant were capable of blocking TNFα binding to receptor. The concentrations of the TNV mAbs in their respective cell supernatants were first determined by standard ELISA analysis for human IgG. A recombinant p55 TNF receptor/IgG fusion protein, p55-sf2, was then coated on EIA plates and ¹²⁵I-labeled TNFα allowed to bind to the p55 receptor in the presence of varying amounts of TNV mAbs. As shown in FIG. 1, all but one (TNV122) of the eight TNV mAbs efficiently blocked TNFα binding to p55 receptor. In fact, the TNV mAbs appeared to be more effective at inhibiting TNFα binding than cA2 positive control mAb that had been spiked into negative control hybridoma supernatant. These results were interpreted as indicating that it was highly likely that the TNV mAbs would block TNFα bioactivity in cell-based assays and in vivo and therefore additional analyses were warranted.

### DNA Sequence Analysis.

Confirmation that the RNAs Encode Human mAbs.

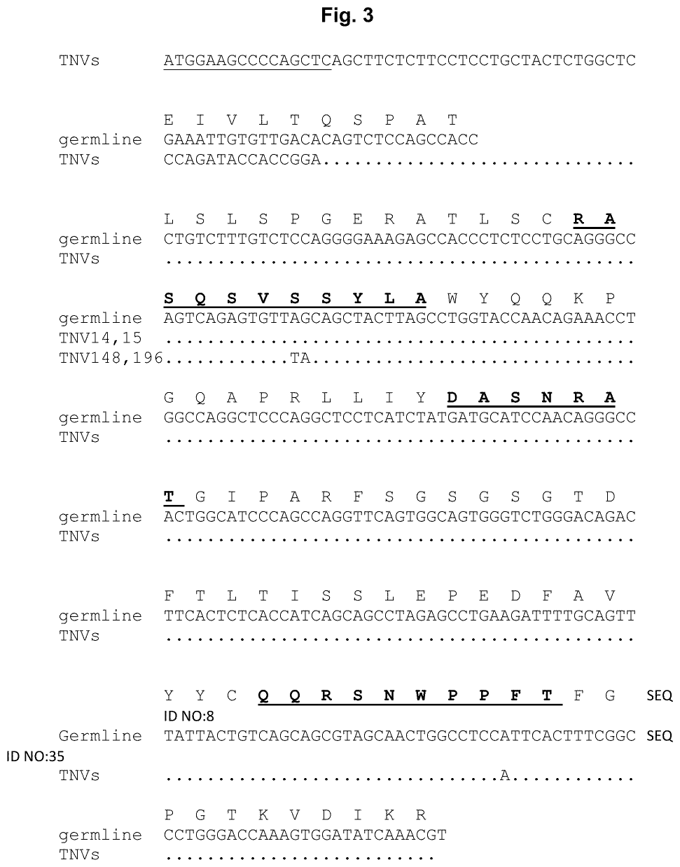

As a first step in characterizing the seven TNV mAbs (TNV14, TNV15, TNV32, TNV86, TNV118, TNV148, and TNV196) that showed TNFα-blocking activity in the receptor binding assay, total RNA was isolated from the seven hybridoma cell lines that produce these mAbs. Each RNA sample was then used to prepare human antibody heavy or light chain cDNA that included the complete signal sequence, the complete variable region sequence, and part of the constant region sequence for each mAb. These cDNA products were then amplified in PCR reactions and the PCR-amplified DNA was directly sequenced without first cloning the fragments. The heavy chain cDNAs sequenced were >90% identical to one of the five human germline genes present in the mice, DP-46 (FIG. 2A-B). Similarly, the light chain cDNAs sequenced were either 100% or 98% identical to one of the human germline genes present in the mice (FIG. 3). These sequence results confirmed that the RNA molecules that were transcribed into cDNA and sequenced encoded human antibody heavy chains and human antibody light chains. It should be noted that, because the variable regions were PCR-amplified using oligonucleotides that map to the 5′ end of the signal sequence coding sequence, the first few amino acids of the signal sequence may not be the actual sequence of the original TNV translation products, but they do represent the actual sequences of the recombinant TNV mAbs.

Unique Neutralizing mAbs.

Analyses of the cDNA sequences for the entire variable regions of both heavy and light chains for each mAb revealed that TNV32 is identical to TNV15, TNV118 is identical to TNV14, and TNV86 is identical to TNV148. The results of the receptor binding assay were consistent with the DNA sequence analyses, i.e. both TNV86 and TNV148 were approximately 4-fold better than both TNV118 and TNV14 at blocking TNF binding. Subsequent work was therefore focused on only the four unique TNV mAbs, TNV14, TNV15, TNV148, and TNV196.

Relatedness of the Four mAbs

The DNA sequence results revealed that the genes encoding the heavy chains of the four TNV mAbs were all highly homologous to each other and appear to have all derived from the same germline gene, DP-46 (FIG. 2A-B). In addition, because each of the heavy chain CDR3 sequences are so similar and of the same length, and because they all use the J6 exon, they apparently arose from a single VDJ gene rearrangement event that was then followed by somatic changes that made each mAb unique. DNA sequence analyses revealed that there were only two distinct light chain genes among the four mAbs (FIG. 3). The light chain variable region coding sequences in TNV14 and TNV15 are identical to each other and to a representative germline sequence of the Vg/38K family of human kappa chains. The TNV148 and TNV light chain coding sequences are identical to each other but differ from the germline sequence at two nucleotide positions (FIG. 3).

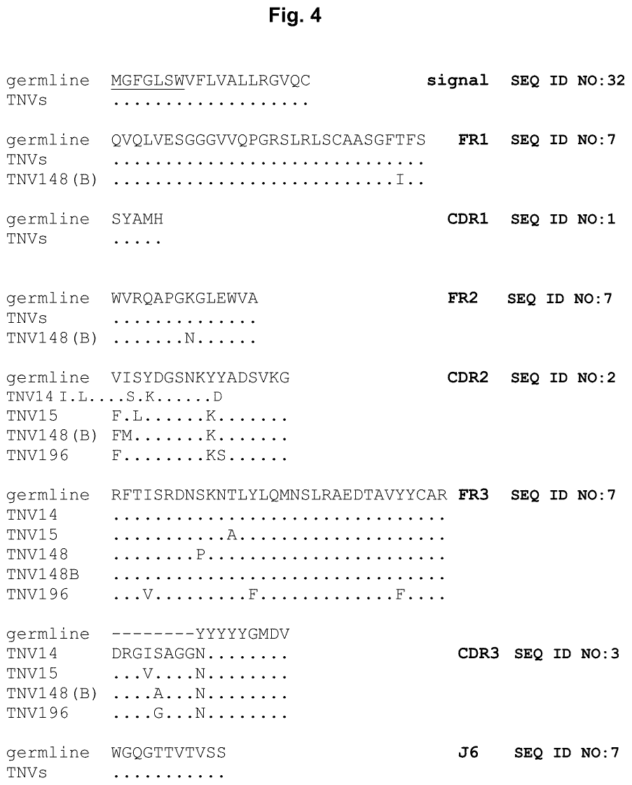

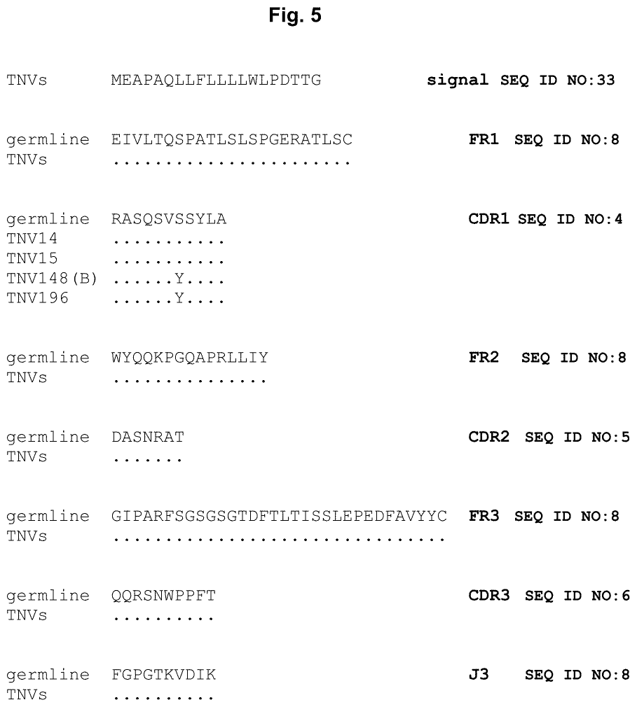

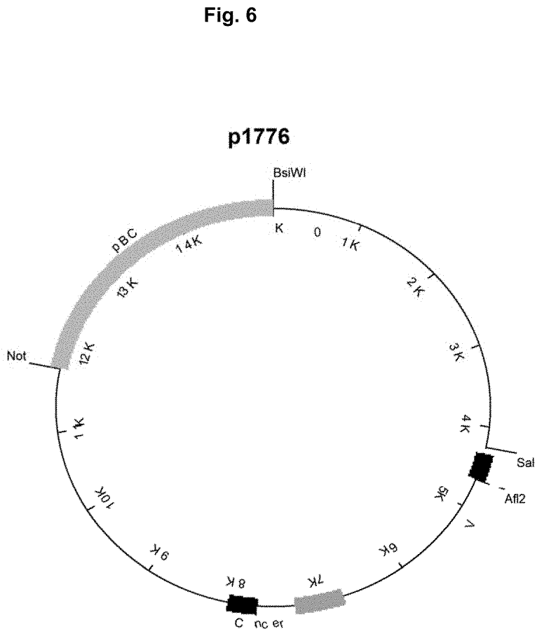

The deduced amino acid sequences of the four mAbs revealed the relatedness of the actual mAbs. The four mAbs contain four distinct heavy chains (FIG. 4) but only two distinct light chains (FIG. 5). Differences between the TNV mAb sequences and the germline sequences were mostly confined to CDR domains but three of the mAb heavy chains also differed from the germline sequence in the framework regions (FIG. 4). Compared to the DP-46 germline-encoded Ab framework regions, TNV14 was identical, TNV15 differed by one amino acid, TNV148 differed by two amino acids, and TNV196 differed by three amino acids.

Cloning of cDNAs, Site-specific Mutagenesis, and Assembly of Final Expression Plasmids. Cloning of cDNAs. Based on the DNA sequence of the PCR-amplified variable regions, new oligonucleotides were ordered to perform another round of PCR amplification for the purpose of adapting the coding sequence to be cloned into expression vectors. In the case of the heavy chains, the products of this second round of PCR were digested with restriction enzymes BsiWI and BstBI and cloned into plasmid vector L28 (plasmid identification numbers shown in Table 2). In the case of the light chains, the second-round PCR products were digested with SalI and AfIII and cloned into plasmid vector pBC. Individual clones were then sequenced to confirm that their sequences were identical to the previous sequence obtained from direct sequencing of PCR products, which reveals the most abundant nucleotide at each position in a potentially heterogeneous population of molecules.

Site-specific Mutagenesis to Change TNV148. mAbs TNV148 and TNV196 were being consistently observed to be four-fold more potent than the next best mAb (TNV14) at neutralizing TNFα bioactivity. However, as described above, the TNV148 and TNV196 heavy chain framework sequences differed from the germline framework sequences. A comparison of the TNV148 heavy chain sequence to other human antibodies indicated that numerous other human mAbs contained an Ile residue at position 28 in framework 1 (counting mature sequence only) whereas the Pro residue at position 75 in framework 3 was an unusual amino acid at that position.

A similar comparison of the TNV196 heavy chain suggested that the three amino acids by which it differs from the germline sequence in framework 3 may be rare in human mAbs. There was a possibility that these differences may render TNV148 and TNV196 immunogenic if administered to humans. Because TNV148 had only one amino acid residue of concern and this residue was believed to be unimportant for TNFα binding, a site-specific mutagenesis technique was used to change a single nucleotide in the TNV148 heavy chain coding sequence (in plasmid p1753) so that a germline Ser residue would be encoded in place of the Pro residue at position 75. The resulting plasmid was termed p1760 (see Table 2). The resulting gene and mAb were termed TNV148B to distinguish it from the original TNV148 gene and mAb (see FIG. 5).

Assembly of Final Expression Plasmids. New antibody expression vectors were prepared that were based on the 12B75 heavy chain and light chain genes previously cloned as genomic fragments. Although different TNV expression plasmids were prepared (see Table 2), in each case the 5′ flanking sequences, promoter, and intron enhancer derived from the respective 12B75 genes. For the light chain expression plasmids, the complete J-C intron, constant region coding sequence and 3′ flanking sequence were also derived from the 12B75 light chain gene. For the heavy chain expression plasmids that resulted in the final production cell lines (p1781 and p1783, see below), the human IgG1 constant region coding sequences derived from Centocor's previously-used expression vector (p104). Importantly, the final production cell lines reported here express a different allotype (Gm(f+)) of the TNV mAbs than the original, hybridoma-derived TNV mAbs (G1m(z)). This is because the 12B75 heavy chain gene derived from the GenPharm mice encodes an Arg residue at the C-terminal end of the CH1 domain whereas Centocor's IgG1 expression vector p104 encodes a Lys residue at that position. Other heavy chain expression plasmids (e.g. p1786 and p1788) were prepared in which the J-C intron, complete constant region coding sequence and 3′ flanking sequence were derived from the 12B75 heavy chain gene, but cell lines transfected with those genes were not selected as the production cell lines. Vectors were carefully designed to permit one-step cloning of future PCR-amplified V regions that would result in final expression plasmids.

PCR-amplified variable region cDNAs were transferred from L28 or pBC vectors to intermediate-stage, 12B75-based vectors that provided the promoter region and part of the J-C intron (see Table 2 for plasmid identification numbers). Restriction fragments that contained the 5′ half of the antibody genes were then transferred from these intermediate-stage vectors to the final expression vectors that provided the 3′ half of the respective genes to form the final expression plasmids (see Table 2 for plasmid identification numbers).

Cell Transfections and Subcloning. Expression plasmids were either linearized by restriction digest or the antibody gene inserts in each plasmid were purified away from the plasmid backbones. Sp2/0 and 653 mouse myeloma cells were transfected with the heavy and light chain DNA by electroporation. Fifteen different transfections were done, most of which were unique as defined by the Ab, specific characteristics of the Ab genes, whether the genes were on linearized whole plasmids or purified gene inserts, and the host cell line (summarized in Table 4). Cell supernatants from clones resistant to mycophenolic acid were assayed for the presence of human IgG by ELISA and quantitated using purified rTNV148B as a reference standard curve.

Highest-Producing rTNV148B Cell Lines

Ten of the best-producing 653 parental lines from rTNV148B transfection 2 (produced 5-10 :g/ml in spent 24-well cultures) were subcloned to screen for higher-producing cell lines and to prepare a more homogeneous cell population. Two of the subclones of the parental line 2.320, 2.320-17 and 2.320-20, produced approximately 50 :g/ml in spent 24-well cultures, which was a 5-fold increase over their parental line. A second round of subcloning of subcloned lines 2.320-17 and 2.320-20 led

The identification numbers of the heavy and light chain plasmids that encode each mAb are shown. In the case of transfections done with purified mAb gene inserts, plasmid p13 (pSV2gpt) was included as a source of the gpt selectable marker. The heavy chain constant regions were encoded either by the same human IgG1 expression vector used to encode Remicade (‘old’) or by the constant regions contained within the 12B75 (GenPharm/Medarex) heavy chain gene (‘new’). H1/L2 refers to a “novel” mAb made up of the TNV14 heavy chain and the TNV148 light chain. Plasmids p1783 and p1801 differ only by how much of the J-C intron their heavy chain genes contain. The transfection numbers, which define the first number of the generic names for cell clones, are shown on the right. The rTNV148B-producing cell lines C466 (A, B, C, D) and C467A described here derived from transfection number 2 and 1, respectively. The rTNV14-producing cell line C476A derived from transfection number 3.

TABLE 4
Summary of Cell Transfections.

| Transfection no. | Plasmids | HC | | DNA | |
|---|---|---|---|---|---|
| mAb | HC/LC/gpt | vector | format | Sp2/0 | 653 |
| rTNV148B | 1783/1776 | old | linear | 1 | 2 |
| rTNV14 | 1781/1775 | old | linear | 3 | — |
| rTNV148B | 1788/1776/13 | new | insert | 4, 6 | 5, 7 |
| rTNV14 | 1786/1775/13 | new | insert | 8, 10 | 9, 11 |
| rTNV148 | 1787/1776/13 | new | insert | 12 | 17 |
| rH1/L2 | 1786/1776/13 | new | insert | 13 | 14 |
| rTNV148B | 1801/1776 | old | linear | 16 | |

ELISA assays on spent 24-well culture supernatants indicated that these second-round subclones all produced between 98 and 124 :g/ml, which was at least a 2-fold increase over the first-round subclones. These 653 cell lines were assigned C code designations as shown in Table 5.

Three of the best-producing Sp2/0 parental lines from rTNV148B transfection 1 were subcloned. Two rounds of subcloning of parental line 1.73 led to the identification of a clone that produced 25 :g/ml in spent 24-well cultures. This Sp2/0 cell line was designated C467A (Table 5).

Highest-Producing rTNV14 Cell Lines

Three of the best-producing Sp2/0 parental lines from rTNV14 transfection 3 were subcloned once. Subclone 3.27-1 was found to be the highest-producer in spent 24-well cultures with a production of 19 :g/ml. This cell line was designated C476A (Table 5).

The first digit of the original clone names indicates which transfection the cell line derived from. All of the C-coded cell lines reported here were derived from transfections with heavy and light chain whole plasmids that had been linearized with restriction enzymes.

TABLE 5
Summary of Selected Production Cell Lines and their C codes.

| | Original | | Spent 24-well | | |
|---|---|---|---|---|---|
| mAb | Clone Name | C code | Host Cell | Production | |
| rTNV148B | 2.320-17-36 | C466A | 653 | 103: | g/ml |
| | 2.320-20-111 | C466B | 653 | 102: | g/ml |
| | 2.320-17-4 | C466C | 653 | 98: | g/ml |
| | 2.320-20-99 | C466D | 653 | 124: | g/ml |
| | 1.73-12-122 | C467A | Sp2/0 | 25: | g/ml |
| rTNV14 | 3.27-1 | C476A | Sp2/0 | 19: | g/ml |

Characterization of Subcloned Cell Lines

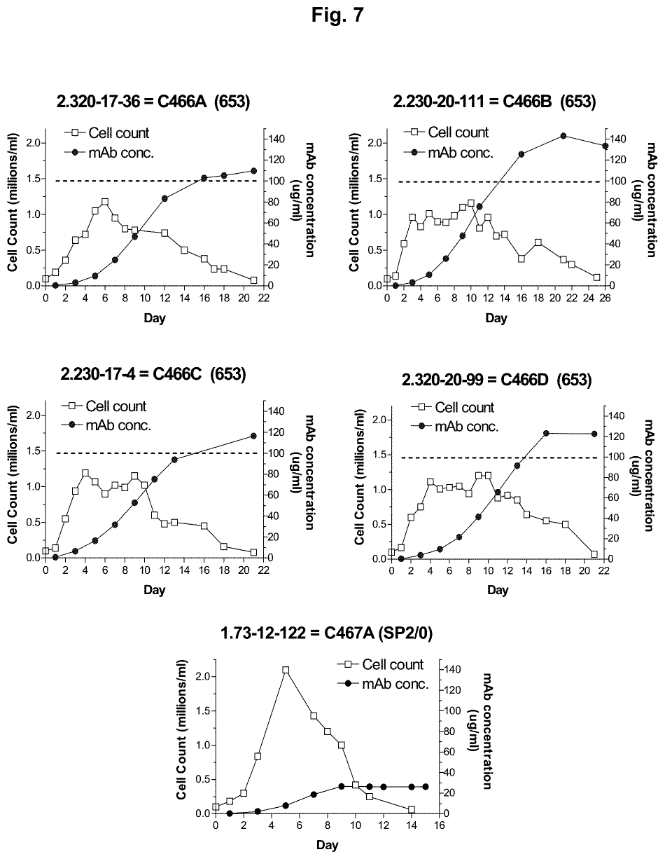

To more carefully characterize cell line growth characteristics and determine mAb-production levels on a larger scale, growth curves analyses were performed using T75 cultures. The results showed that each of the four C466 series of cell lines reached peak cell density between 1.0×10⁶ and 1.25×10⁶ cells/ml and maximal mAb accumulation levels of between 110 and 140 :g/ml (FIG. 7). In contrast, the best-producing Sp2/0 subclone, C467A, reached peak cell density of 2.0×10⁶ cells/ml and maximal mAb accumulation levels of 25 :g/ml (FIG. 7). A growth curve analysis was not done on the rTNV14-producing cell line, C476A.

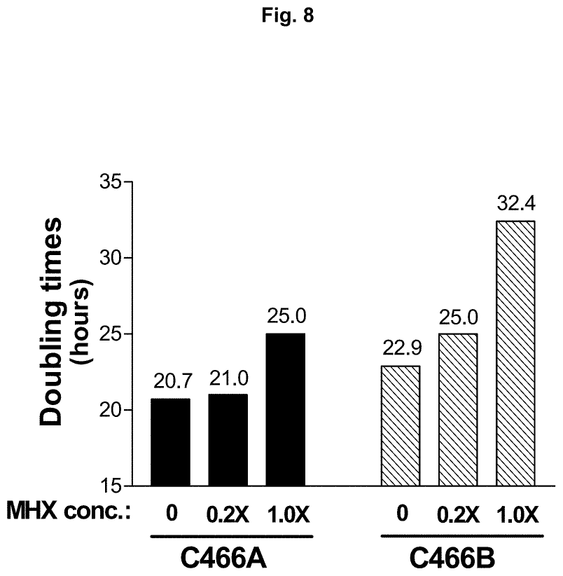

An additional growth curve analysis was done to compare the growth rates in different concentrations of MHX selection. This comparison was prompted by recent observations that C466 cells cultured in the absence of MHX seemed to be growing faster than the same cells cultured in the normal amount of MHX (1×). Because the cytotoxic concentrations of compounds such as mycophenolic acid tend to be measured over orders of magnitude, it was considered possible that the use of a lower concentration of MHX might result in significantly faster cell doubling times without sacrificing stability of mAb production. Cell lines C466A and C466B were cultured either in: no MHX, 0.2×MHX, or 1×MHX. Live cell counts were taken at 24-hour intervals for 7 days. The results did reveal an MI-IX concentration-dependent rate of cell growth (FIG. 8). Cell line C466A showed a doubling time of 25.0 hours in 2×MHX but only 20.7 hours in no MHX. Similarly, cell line C466B showed a doubling time of 32.4 hours in 1×MHX but only 22.9 hours in no MHX. Importantly, the doubling times for both cell lines in 0.2×MHX were more similar to what was observed in no MHX than in 1×MHX (FIG. 8). This observation raises the possibility than enhanced cell performance in bioreactors, for which doubling times are an important parameter, could be realized by using less MHX. However, although stability test results (see below) suggest that cell line C466D is capable of stably producing rTNV148B for at least 60 days even with no MHX present, the stability test also showed higher mAb production levels when the cells were cultured in the presence of MHX compared to the absence of MHX.

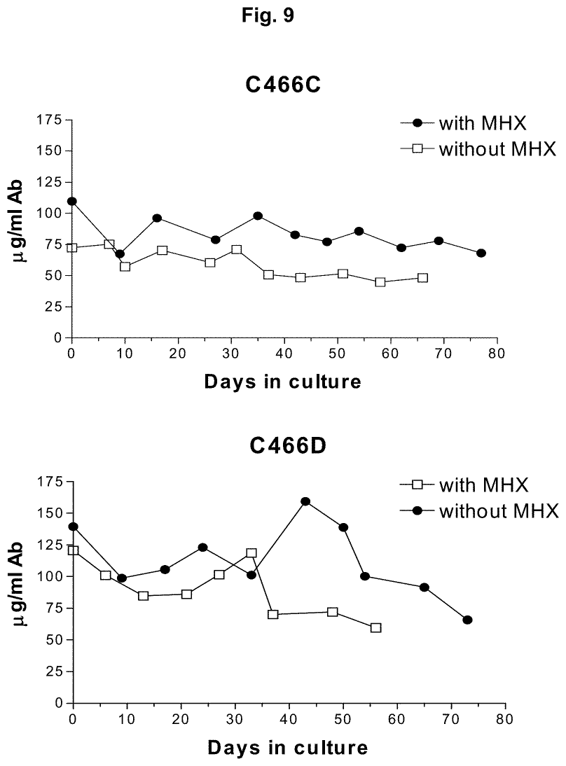

To evaluate mAb production from the various cell lines over a period of approximately 60 days, stability tests were performed on cultures that either contained, or did not contain, MHX selection. Not all of the cell lines maintained high mAb production. After just two weeks of culture, clone C466A was producing approximately 45% less than at the beginning of the study. Production from clone C466B also appeared to drop significantly. However, clones C466C and C466D maintained fairly stable production, with C466D showing the highest absolute production levels (FIG. 9).

### Conclusion

From an initial panel of eight human mAbs against human TNFα, TNV148B was selected as preferred based on several criteria that included protein sequence and TNF neutralization potency, as well as TNV14. Cell lines were prepared that produce greater than 100 :g/ml of rTNV148B and 19 :g/ml rTNV14.

### EXAMPLE 5

### Arthritic Mice Study Using Anti-TNF Antibodies and Controls Using Single Bolus Injection

At approximately 4 weeks of age the Tg197 study mice were assigned, based on gender and body weight, to one of 9 treatment groups and treated with a single intraperitoneal bolus dose of Dulbecco's PBS (D-PBS) or an anti-TNF antibody of the present invention (TNV14, TNV148 or TNV196) at either 1 mg/kg or 10 mg/kg.

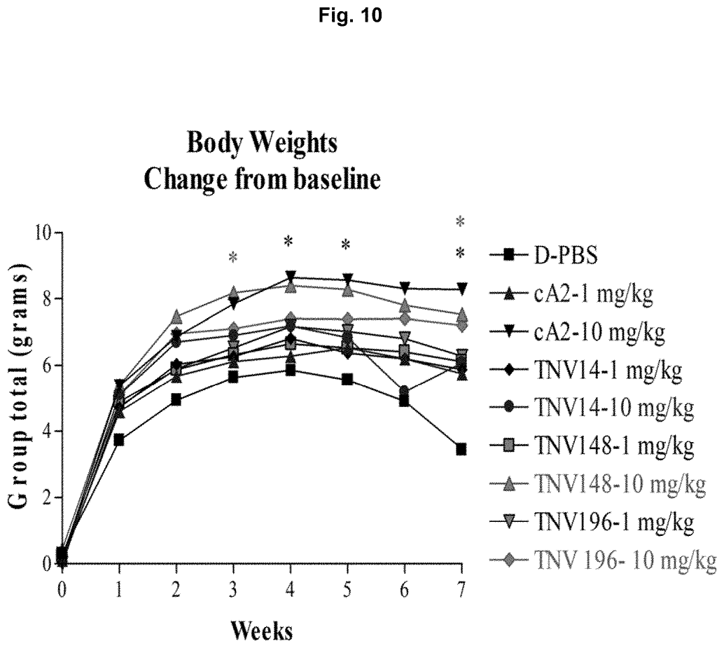

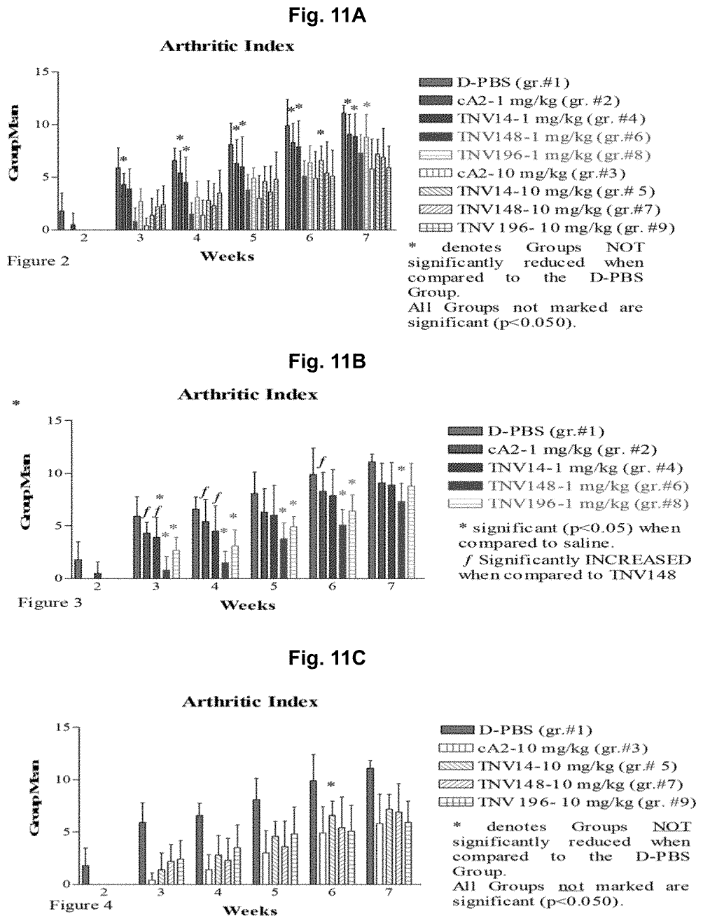

RESULTS: When the weights were analyzed as a change from pre-dose, the animals treated with 10 mg/kg cA2 showed consistently higher weight gain than the D-PBS-treated animals throughout the study. This weight gain was significant at weeks 3-7. The animals treated with 10 mg/kg TNV148 also achieved significant weight gain at week 7 of the study. (See FIG. 10).

FIG. 11A-C represent the progression of disease severity based on the arthritic index. The 10 mg/kg cA2-treated group's arthritic index was lower than the D-PBS control group starting at week 3 and continuing throughout the remainder of the study (week 7). The animals treated with 1 mg/kg TNV14 and the animals treated with 1 mg/kg cA2 failed to show significant reduction in AI after week 3 when compared to the D-PBS-treated Group. There were no significant differences between the 10 mg/kg treatment groups when each was compared to the others of similar dose (10 mg/kg cA2 compared to 10 mg/kg TNV14, 148 and 196). When the 1 mg/kg treatment groups were compared, the 1 mg/kg TNV148 showed a significantly lower AI than 1 mg/kg cA2 at 3, 4 and 7 weeks. The 1 mg/kg TNV148 was also significantly lower than the 1 mg/kg TNV14-treated Group at 3 and 4 weeks. Although TNV196 showed significant reduction in AI up to week 6 of the study (when compared to the D-PBS-treated Group), TNV148 was the only 1 mg/kg treatment that remained significant at the conclusion of the study.

### EXAMPLE 6

### Arthritic Mice Study Using Anti-TNF Antibodies and Controls as Multiple Bolus Doses

At approximately 4 weeks of age the Tg197 study mice were assigned, based on body weight, to one of 8 treatment groups and treated with a intraperitoneal bolus dose of control article (D-PBS) or antibody (TNV14, TNV148) at 3 mg/kg (week 0). Injections were repeated in all animals at weeks 1, 2, 3, and 4. Groups 1-6 were evaluated for test article efficacy. Serum samples, obtained from animals in Groups 7 and 8 were evaluated for immune response induction and pharmacokinetic clearance of TNV14 or TNV148 at weeks 2, 3 and 4.

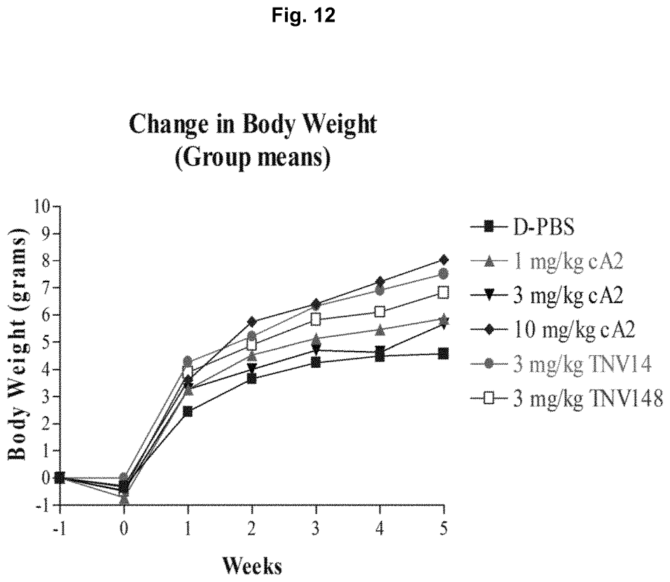

RESULTS: No significant differences were noted when the weights were analyzed as a change from pre-dose. The animals treated with 10 mg/kg cA2 showed consistently higher weight gain than the D-PBS-treated animals throughout the study. (See FIG. 12).

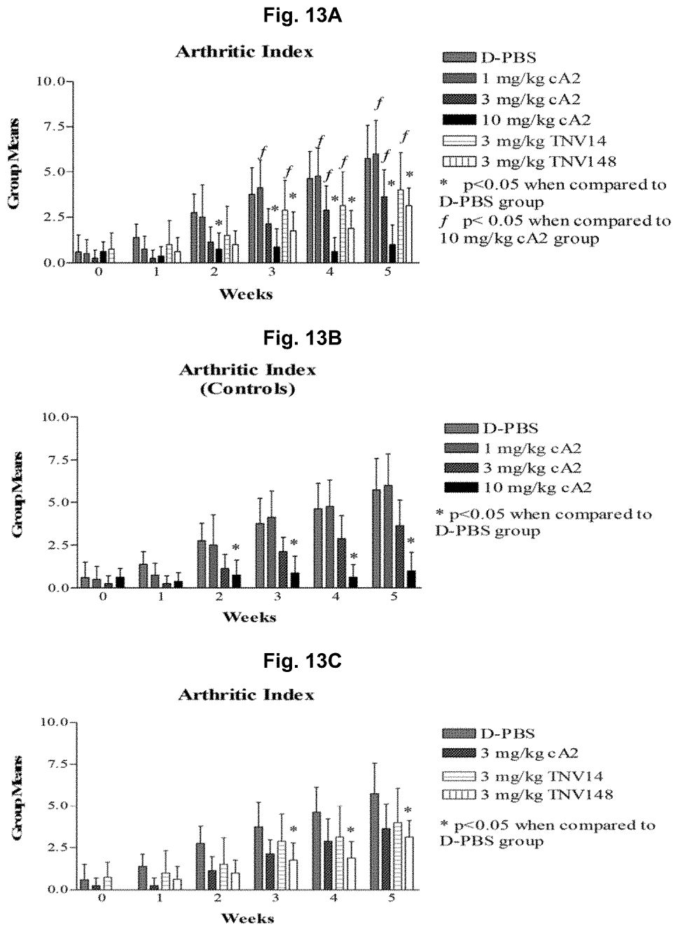

FIG. 13A-C represent the progression of disease severity based on the arthritic index. The 10 mg/kg cA2-treated group's arthritic index was significantly lower than the D-PBS control group starting at week 2 and continuing throughout the remainder of the study (week 5). The animals treated with 1 mg/kg or 3 mg/kg of cA2 and the animals treated with 3 mg/kg TNV14 failed to achieve any significant reduction in AI at any time throughout the study when compared to the d-PBS control group. The animals treated with 3 mg/kg TNV148 showed a significant reduction when compared to the d-PBS-treated group starting at week 3 and continuing through week 5. The 10 mg/kg cA2-treated animals showed a significant reduction in AI when compared to both the lower doses (1 mg/kg and 3 mg/kg) of cA2 at weeks 4 and 5 of the study and was also significantly lower than the TNV14-treated animals at weeks 3-5. Although there appeared to be no significant differences between any of the 3 mg/kg treatment groups, the AI for the animals treated with 3 mg/kg TNV14 were significantly higher at some time points than the 10 mg/kg whereas the animals treated with TNV148 were not significantly different from the animals treated with 10 mg/kg of cA2.

### EXAMPLE 7

### Arthritic Mice Study Using Anti-TNF Antibodies and Controls as Single Intraperitoneal Bolus Dose

At approximately 4 weeks of age the Tg197 study mice were assigned, based on gender and body weight, to one of 6 treatment groups and treated with a single intraperitoneal bolus dose of antibody (cA2, or TNV148) at either 3 mg/kg or 5 mg/kg. This study utilized the D-PBS and 10 mg/kg cA2 control Groups.

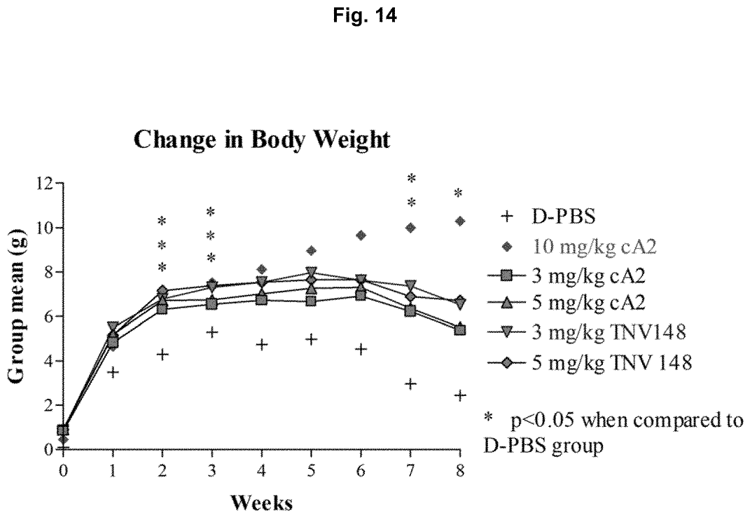

When the weights were analyzed as a change from pre-dose, all treatments achieved similar weight gains. The animals treated with either 3 or 5 mg/kg TNV148 or 5 mg/kg cA2 gained a significant amount of weight early in the study (at weeks 2 and 3). Only the animals treated with TNV148 maintained significant weight gain in the later time points. Both the 3 and 5 mg/kg TNV148-treated animals showed significance at 7 weeks and the 3 mg/kg TNV148 animals were still significantly elevated at 8 weeks post injection. (See FIG. 14).

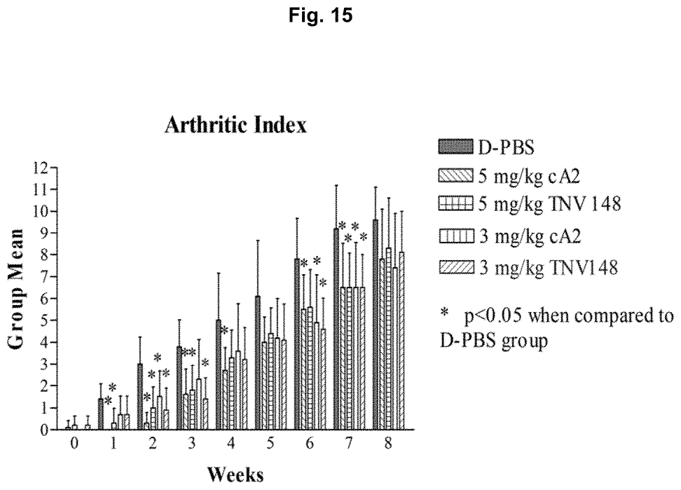

FIG. 15 represents the progression of disease severity based on the arthritic index. All treatment groups showed some protection at the earlier time points, with the 5 mg/kg cA2 and the 5 mg/kg TNV148 showing significant reductions in AI at weeks 1-3 and all treatment groups showing a significant reduction at week 2. Later in the study the animals treated with 5 mg/kg cA2 showed some protection, with significant reductions at weeks 4, 6 and 7. The low dose (3 mg/kg) of both the cA2 and the TNV148 showed significant reductions at 6 and all treatment groups showed significant reductions at week 7. None of the treatment groups were able to maintain a significant reduction at the conclusion of the study (week 8). There were no significant differences between any of the treatment groups (excluding the saline control group) at any time point.

### EXAMPLE 8

### Arthritic Mice Study Using Anti-TNF Antibodies and Controls as Single Intraperitoneal Bolus Dose Between Anti-TNF Antibody and Modified Anti-TNF Antibody

To compare the efficacy of a single intraperitoneal dose of TNV148 (derived from hybridoma cells) and rTNV148B (derived from transfected cells). At approximately 4 weeks of age the Tg197 study mice were assigned, based on gender and body weight, to one of 9 treatment groups and treated with a single intraperitoneal bolus dose of Dulbecco=S PBS (D-PBS) or antibody (TNV148, rTNV148B) at 1 mg/kg.

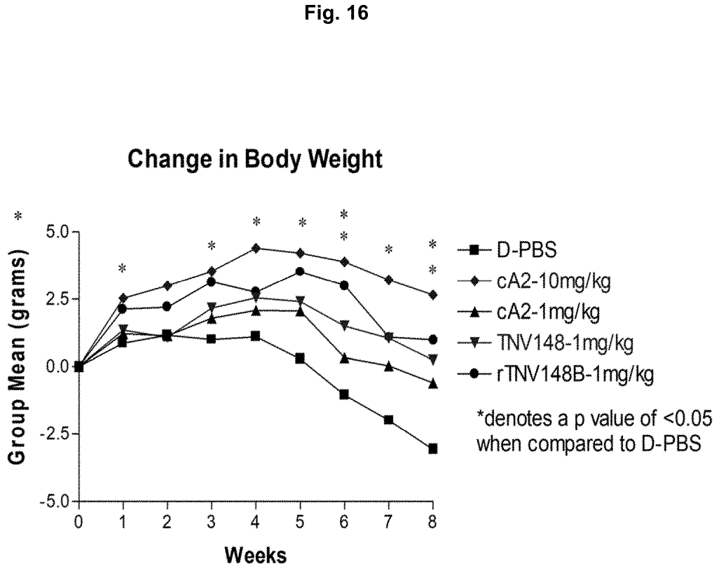

When the weights were analyzed as a change from pre-dose, the animals treated with 10 mg/kg cA2 showed a consistently higher weight gain than the D-PBS-treated animals throughout the study. This weight gain was significant at weeks 1 and weeks 3-8. The animals treated with 1 mg/kg TNV148 also achieved significant weight gain at weeks 5, 6 and 8 of the study. (See FIG. 16).

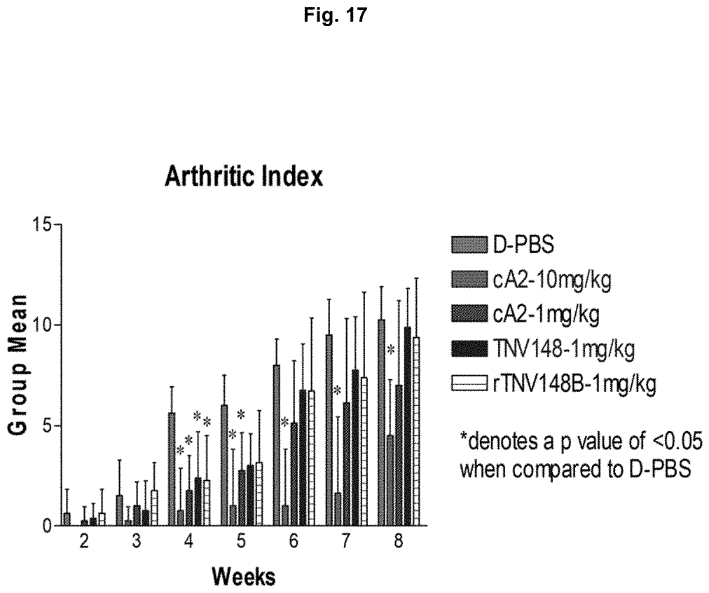

FIG. 17 represents the progression of disease severity based on the arthritic index. The 10 mg/kg cA2-treated group's arthritic index was lower than the D-PBS control group starting at week 4 and continuing throughout the remainder of the study (week 8). Both of the TNV148-treated Groups and the 1 mg/kg cA2-treated Group showed a significant reduction in AI at week 4. Although a previous study (P-099-017) showed that TNV148 was slightly more effective at reducing the Arthritic Index following a single 1 mg/kg intraperitoneal bolus, this study showed that the AI from both versions of the TNV antibody-treated groups was slightly higher. Although (with the exception of week 6) the 1 mg/kg cA2-treated Group was not significantly increased when compared to the 10 mg/kg cA2 group and the TNV148-treated Groups were significantly higher at weeks 7 and 8, there were no significant differences in AI between the 1 mg/kg cA2, 1 mg/kg TNV148 and 1 mg/kg TNV148B at any point in the study.

### EXAMPLE 9

### GO-VIVA—A Multicenter, Open-Label Trial of Intravenous Golimumab, a Human Anti-TNFα Antibody, in Pediatric Subjects with Active Polyarticular Course Juvenile Idiopathic Arthritis Despite Methotrexate Therapy

### Synopsis

Golimumab is a fully human monoclonal antibody (mAb) which binds to human tumor necrosis factor alpha (TNFα) with high affinity and specificity and neutralizes TNFα bioactivity. TNFα is a key inflammatory mediator, with high levels of TNFα implicated in the pathophysiology of diseases such as rheumatoid arthritis (RA) and juvenile idiopathic arthritis (JIA). SIMPONI® (golimumab) for intravenous (IV) use is being developed by the Sponsor to offer an alternative route of administration (compared with other available anti-TNFα agents) and a convenient dose regimen (ie, every 8 week [q8w] administration) for patients with polyarticular JIA (pJIA).

### Objectives and Hypothesis

### Primary Objective

The primary objective of this study is to assess the pharmacokinetics (PK) following intravenously administered golimumab in subjects (ages 2 to less than 18 years) with pJIA manifested by ≥5 joints with active arthritis despite methotrexate (MTX) therapy for ≥2 months.

### Secondary Objectives

The secondary objectives of this study are to evaluate IV golimumab in subjects with pJIA with respect to PK, efficacy (relief of signs and symptoms, physical function, and quality of life), safety (adverse events [AEs], serious AEs [SAES], and assessment of laboratory parameters), and immunogenicity (antibodies to golimumab).

### Hypothesis

No formal hypothesis testing is planned in this study.

### Overview of Study Design

This is a Phase 3, open-label, single-arm, multicenter study to evaluate the PK, safety, and efficacy of IV golimumab in subjects with active pJIA despite current treatment with MTX. The study population will comprise subjects with pJIA receiving MTX, ages 2 to less than 18 years, with at least a 3-month history of pJIA, and active arthritis in ≥5 joints. Approximately 120 subjects will be enrolled at Week 0 to ensure that approximately 100 subjects remain in the study at Week 52. Enrollment patterns are expected to yield a subject population of approximately 10% aged 2 to up to 6 years, approximately 20% aged 6 to up to 12 years, and approximately 70% aged 12 to less than 18 years.

All subjects will receive 80 mg/m² golimumab as an IV infusion (over 30±10 minutes) at Weeks 0, 4, and q8w (±3 days) through Week 28 and q8w (±1 week) thereafter (maximum single dose 240 mg [maximum body surface area (BSA) 3.0 m²×80 mg/m²]). Commercial MTX is to be administered at a stable dose of 10-30 mg/m²/week in subjects with BSA<1.67 m² or a stable minimum dose of 15 mg/week in subjects with BSA≥1.67 m² through Week 28 (unless lower doses of MTX are administered for documented safety reasons or unless documented country or site regulations prohibit dose of 15 mg/week or above in subjects with BSA≥1.67 m²). Subjects who complete the study at Week 52 will have the option to enter into the long-term extension (LTE) phase of the study. During the LTE, all subjects will continue to receive 80 mg/m² IV golimumab q8w (±1 week; maximum single dose 240 mg) through Week 244. All subjects who complete the Week 244 visit are expected to participate in the safety follow-up visit at Week 252. Golimumab after Week 252 (for subjects who have completed the full 252-week study before drug commercialization for pJIA indication has taken place) will be provided until the drug will be approved and marketed for use in pJIA in the country of the subject or for as long as proven beneficial to the child (in cases where commercial drug is not accessible to the subject).

Since this is an open-label study with all subjects receiving the same BSA-based dose of IV golimumab, an external Data Monitoring Committee will not be established.

The end of the study is defined as the last follow-up assessment for the last subject in LTE.

### Subject Population

Study subjects must be 2 to less than 18 years of age with a body weight >15 kg at the time of enrollment.

The onset of disease must have been before the subject's 16th birthday, must be of at least 3 months' duration, and must be active pJIA of one of the following subtypes: rheumatoid factor positive or negative pJIA; systemic JIA with no systemic symptoms for ≥3 months but with polyarthritis for ≥3 months; extended oligoarticular JIA; enthesitis-related arthritis or polyarticular juvenile psoriatic arthritis (PsA).

Subjects must have ≥5 joints with active arthritis as defined by American College of Rheumatology (ACR) criteria at screening and enrollment. Subjects must have active pJIA despite current use of oral, intramuscular, or subcutaneous MTX (for ≥2 months before screening) at a weekly dose of ≥10 mg/m².

### Dosage and Administration

### Golimumab

The study will have 1 active treatment group and all subjects will receive 80 mg/m² golimumab IV infusions at Week 0, Week 4, and q8w (±3 days) through Week 28 and q8w (±1 week) thereafter through Week 244. BSA will be calculated at each visit and the dose of golimumab will be adjusted as needed to maintain the dose at 80 mg/m². BSA will be calculated using the Mosteller equation: BSA (m²)=([height (cm)×weight (kg)]/3600)^{1/2}. The maximum single dose will be golimumab 240 mg.

### Methotrexate

Subjects will receive commercial MTX at least through Week 28 at the same BSA-based dose (10 to 30 mg/m² per week for subjects with BSA<1.67 m² or at least 15 mg/week for subjects with BSA≥1.67 m²) as at time of study entry. Every effort should be made to ensure that subjects remain on the same dose and route of administration of MTX through the Week 28 visit, unless intolerance or AEs due to MTX occur.

Subjects will also receive commercial folic acid ≥5 mg weekly or folinic acid (at half the MTX dose) given the day after the weekly MTX dose. In children <12 years of age, the administration of folic acid or folinic acid will be at the discretion of the physician.

### Efficacy Evaluations and Endpoints

Efficacy evaluations include the following:
Joint evaluations (number of active joints and number of joints with limited range of motion)
Physician Global Assessment of Disease Activity
Childhood Health Assessment Questionnaire (CHAQ; includes the Parent/Subject Assessment of Overall Well-being and Parent/Subject Assessment of Pain)
CRP

No primary efficacy endpoint or major secondary endpoints are planned. Other efficacy endpoints include:
The proportions of subjects who are JIA ACR 30, 50, 70, and 90 responders over time
The change from baseline in CHAQ over time
CRP concentrations over time
The proportion of subjects who have inactive disease over time
The proportion of subjects in clinical remission on medication for pJIA over time
The improvement from baseline in the pJIA core set at each visit
The proportions of subjects who are JIA ACR 30, JIA ACR 50, JIA ACR 70 and JIA ACR 90 responders by disease subtype, and/or age over time through Week 52
The change from baseline in Juvenile Arthritis Disease Activity Score (JADAS) 10, 27, and 71 scores over time
The proportion of subjects who achieve JADAS 10, 27, and 71 minimal disease activity over time

### Pharmacokinetic Evaluations and Endpoints

Serum golimumab concentration will be evaluated at Weeks 0, 4, 8, 12, 20, 28, 52, 100, 148, 196, and 244 and summarized over time. A population PK analysis with data through Week 28 will be performed to characterize the PK of golimumab as well as to identify important covariates of PK in the pediatric population with pJIA.

Golimumab concentrations will be summarized and PK exposure will be evaluated through Week 52 and through the LTE.

The primary endpoint in this study is PK exposure at Week 28 (the trough concentrations at Week 28) and the Bayesian steady-state area under the curve [AUC_{SS}] over one dosing interval of 8 weeks (from population PK modeling and simulation).

The major secondary PK endpoints include:
PK exposure at Week 52 (the trough concentrations at Week 52) and Bayesian AUC_{SS} at Week 52 (from population PK modeling and simulation).

### Safety Evaluations

Safety evaluations include assessments of the following: AEs; infusion reactions; allergic reactions; clinical laboratory tests (hematology, chemistry, and pregnancy testing); vital signs; physical examination; height and body weight; uveitis; and early detection of tuberculosis.

### Immunogenicity Evaluations

Antibodies to golimumab will be evaluated in serum samples collected from all subjects at Weeks 0, 4, 8, 12, 28, 52, 100, 148, 196, and 244.

### Statistical Methods

### Subject Information

Demographics and baseline disease characteristics and prior medication data will be summarized for all subjects enrolled in the study, whether or not they have received study agent administration. Pharmacokinetic data will be summarized for all subjects who had received at least 1 administration of study agent. Efficacy analyses will be summarized for all subjects enrolled in the study. Safety assessments will be summarized for all treated subjects.

### Sample Size

The sample size determination is not based on statistical considerations. The goal is to have a sample size that will be sufficient to build a population PK model and, if feasible, an exposure-response model. Additionally, a sample size that will provide reasonable safety assessments was also taken into consideration. With these considerations, a sample size of approximately 120 subjects has been chosen assuming that if 20 subjects drop out or if they do not provide PK samples, a sample size of approximately 100 subjects will remain in the study at Week 52. This sample size is thought to be sufficient to build a population PK model, given the sparse sampling of PK time points, as well as provide 1 year of safety data from approximately 100 subjects.

### Efficacy Analyses

No primary efficacy endpoint analysis and no major secondary efficacy endpoint analyses are planned.

The following will be summarized for all subjects enrolled in the study:
The proportion of subjects who are JIA ACR 30, 50, 70, and 90 responders over time
The change from baseline in CHAQ over time
CRP concentrations over time
The proportion of subjects who have inactive disease over time
The proportion of subjects in clinical remission on medication for pJIA (ACR criteria) over time
The improvement from baseline in the pJIA core set over time
The proportions of subjects who are JIA ACR 30, 50, 70, and 90 responders by disease subtype, and/or age over time through Week 52
The change from baseline in JADAS 10, 27, and 71 scores over time
The proportion of subjects who achieve JADAS 10, 27, and 71 minimal disease activity over time

### Pharmacokinetic Analyses

The primary objective of this study is to characterize golimumab PK exposure (the trough concentrations at Week 28 and the Bayesian AUC_{SS} over a dosage interval of 8 weeks from population PK modeling and simulation) in the JIA population.

Serum golimumab concentrations will be summarized over time. In addition, a population PK analysis on data through Week 28 will be performed to characterize the PK of golimumab as well as to identify and quantify important covariates of PK in the pediatric population with JIA. Clearance and volume of distribution will be estimated using a nonlinear mixed effects modeling (NONMEM) approach.

### Safety Analyses

Safety will be assessed by evaluating summaries of AEs, clinical laboratory tests, and vital signs findings through Week 252.

### Immunogenicity Analyses

The occurrence and titers of antibodies to golimumab during the study will be summarized over time for all subjects who receive an administration of golimumab and have appropriate samples collected for detection of antibodies to golimumab (ie, subjects with at least 1 sample obtained after their first golimumab administration).

### Pharmacokinetic/Pharmacodynamic Analyses

The relationships between serum golimumab concentration and efficacy will be explored. A suitable PK/pharmacodynamic (PD) model will be explored and developed to describe the exposure-response relationship.

### Time and Events Schedules

TABLE 6
Screening Through Week 52

| | | | | | | | | | | | | |
|---|---|---|---|---|---|---|---|---|---|---|---|---|
| Screening | | | | | | | | | | | | Final Safety |
| Period | Week | Week | Week | Week | Week | Week | Week | Week | Week | Week | Week | Follow-up |
| (−6 weeks) | 0^{a} | 4^{a} | 8^{a} | 12^{a} | 16^{a} | 20^{a} | 24^{a} | 28^{a} | 36^{a} | 44^{a} | 52^{a} | Visit^{b} |

Procedures and Evaluations
Administrative

| | | | | | | | | | | | | | |
|---|---|---|---|---|---|---|---|---|---|---|---|---|---|
| Informed consent/ | X | | | | | | | | | | | | |
| Assent | | | | | | | | | | | | | |
| Medical history/ | X | | | | | | | | | | | | |
| demographic data | | | | | | | | | | | | | |
| Concomitant | X | X | X | X | X | X | X | X | X | X | X | X | X |
| medications collection | | | | | | | | | | | | | |
| Inclusion/exclusion | X | X | | | | | | | | | | | |
| criteria | | | | | | | | | | | | | |

Study Agent

| | | | | | | | | |
|---|---|---|---|---|---|---|---|---|
| IV administration | X | X | X | X | X | X | X | X |
| of study agent | | | | | | | | |

Safety

| | | | | | | | | | | | | | |
|---|---|---|---|---|---|---|---|---|---|---|---|---|---|
| Review of systems | X | X | X | X | X | X | X | X | X | X | X | X | X |
| Physical examination ^{c} | X | | | X | | | X | | | X | | | |
| Body weight measurement | X | X | X | X | X | X | X | X | X | X | X | X | |
| Height measurement | X | X | X | X | X | X | X | X | X | X | X | X | |
| Vital signs | X | X^{d} | X^{d} | X | X^{d} | X | X^{d} | X | X^{d} | X^{d} | X^{d} | X^{d} | X |
| Routine laboratory | X | X | X | | X | | X | | X | X | X | X | X |
| analyses | | | | | | | | | | | | | |
| Hepatitis B | X | | | | | | | | | | | | |
| virus screening | | | | | | | | | | | | | |
| Hepatitis C | X | | | | | | | | | | | | |
| virus screening | | | | | | | | | | | | | |
| QuantiFERON ®-TB | X | | | | | | | | | | | X^{f} | |
| Gold test^{e} | | | | | | | | | | | | | |
| TB evaluation | X | X | X | X | X | X | X | X | X | X | X | X | X |
| (questionnaire) | | | | | | | | | | | | | |
| Chest x-ray^{g} | X | | | | | | | | | | | | |
| Uveitis evaluations^{h} | X | | | | | | X | | | | X | | X |
| Rheumatoid factor | X | | | | | | | | | | | | |
| ANA/Anti- | X | | | | | | | X | | | | X | X |
| dsDNA antibodies | | | | | | | | | | | | | |
| Pregnancy test (serum)ⁱ | X | | | | | | | | | | | | |
| Pregnancy test (urine)ⁱ | | X | X | | X | | X | | X | X | X | X | |
| Infusion reaction | | X | X | | X | | X | | X | X | X | X | |
| evaluation^{j} | | | | | | | | | | | | | |
| Adverse events | X | X | X | X | X | X | X | X | X | X | X | X | X |

Efficacy

| | | | | | | | | | | | | | |
|---|---|---|---|---|---|---|---|---|---|---|---|---|---|
| Joint assessments | X | X | X | X | X | X | X | X | X | X | X | X | X |
| JIA assessments^{k, l} | | X | X | X | X | X | X | X | X | X | X | X | X |
| CRP | X | X | X | X | X | X | X | X | X | X | X | X | X |

Pharmacokinetics

| | | | | | | | | |
|---|---|---|---|---|---|---|---|---|
| Golimumab | 2X | 2X | X | 2X | X | X | X | X |
| concentration^{m, n} | | | | | | | | |

| | |
|---|---|
| Population PK^{o} | ← X^{o} → |

Immunogenicity

| | | | | | | | |
|---|---|---|---|---|---|---|---|
| Antibodies to | X | X | X | X | X | X | X |
| golimumab ⁿ | | | | | | | |

^{a}All scheduled visits should occur within ±3 days of the intended visit through Week 28 and ±1 week after Week 28 through Week 52.
^{b}All subjects who discontinue study agent administration before Week 52 but do not withdraw consent must return to the study site for a final safety visit approximately 8 weeks after the last infusion (Section 10.2).
^{c} Includes skin examination at every physical examination and Tanner staging approximately every 6 months.
^{d}Vital signs should be taken pre-infusion; at 15 and 30 minutes (15-minute intervals during the infusion); and at 60 and 90 minutes (during the 1-hour observation period following the infusion).
^{e}Tuberculin skin tests should also be performed in countries where the QuantiFERON ®-TB Gold test is not approved/registered in that country or the tuberculin skin test is mandated by local Health Authorities.
^{f}Testing is not required for subjects with a history of latent TB and ongoing treatment for latent TB or documentation of having completed adequate treatment.
^{g}Chest x-ray screening as per local and country regulations for initiation of immunosuppressive agents in children with JIA who are at risk of TB.
^{h}Evaluations (based on physical examination and interview) should be performed by the investigator at least every 6 months in all subjects. In addition, all subjects are required to have slit lamp evaluations performed by an ophthalmologist/optometrist during the study at intervals (based on JIA subtype, ANA test results, age at JIA onset, and JIA duration) as specified.
ⁱAll female subjects of childbearing potential (ie, post-menarche) must test negative for pregnancy during screening and at all visits prior to study drug administration.
^{j}Subjects will be observed for at least 60 minutes after the administration of study agent for symptoms of an infusion reaction.
^{k}JIA assessments include the following: Physician Global Assessment of Disease Activity, Childhood Health Assessment Questionnaire (CHAQ), and duration of morning stiffness. CHAQ should be completed before any tests, procedures, or other consultations for that visit to prevent influencing subjects' perceptions.
^{l} CHAQ to be completed by the parent or caregiver; preferably the same parent or caregiver should complete at every visit. Subjects who are 15 to <18 years of age at study entry may complete the assessment jointly with the parent/caregiver.
^{m}At the Weeks 0, 4, and 12 visits, 2 samples for serum golimumab concentrations (indicated by “2X” in the schedule above) will be collected: 1 sample will be collected immediately prior to the infusion and the other collected approximately 1 hour (eg, ±10 minutes) after the end of the infusion. For each of the remaining visits, only 1 sample for serum golimumab will be collected, which should be collected prior to the infusion if an infusion of the study agent is administered at that visit. Post-infusion samples should be drawn from a different arm than the IV infusion line, or the IV infusion line must be flushed and cleared of any residual medication that may be remaining and 1 mL of blood should be drawn and discarded prior to obtaining the sample if using the same access line as was used for drug administration.
ⁿ The same serum samples may be used for the measurement of golimumab concentration and detection of antibodies to golimumab. For visits with study agent administration, all blood samples for assessing golimumab concentration and antibodies to golimumab MUST be collected BEFORE the administration of the study agent.
^{o}One additional sample for serum golimumab concentration for population PK will be collected from all subjects at any time between Weeks 0 and 8 other than at the time of the Week 0, Week 4, and Week 8 visits; this sample must be collected at least 24 hours prior to or after a study agent administration and must not be collected at a regularly scheduled visit (eg, Week 8).
Abbreviations: ANA = antinuclear antibodies; CHAQ = Childhood Health Assessment Questionnaire; CRP = C-reactive protein; dsDNA = double-stranded deoxyribonucleic acid; IV = intravenous; PK = pharmacokinetic; TB = tuberculosis.

TABLE 7
From Week 60 Through Week 156 (Long-term Extension)

| | | | | | | | | | | | | | |
|---|---|---|---|---|---|---|---|---|---|---|---|---|---|
| | | | | | | | | | | | | | Final Safety |
| Week | Week | Week | Week | Week | Week | Week | Week | Week | Week | Week | Week | Week | Follow-up |
| 60 ^{a} | 68 ^{a} | 76 ^{a} | 84 ^{a} | 92 ^{a} | 100 ^{a} | 108 ^{a} | 116 ^{a} | 124 ^{a} | 132 ^{a} | 140 ^{a} | 148 ^{a} | 156 ^{a} | Visit^{b} |

Procedures and Evaluations
Administrative

| | | | | | | | | | | | | | | |
|---|---|---|---|---|---|---|---|---|---|---|---|---|---|---|
| Concomitant | X | X | X | X | X | X | X | X | X | X | X | X | X | X |
| medications collection | | | | | | | | | | | | | | |

Study Agent

| | | | | | | | | | | | | | |
|---|---|---|---|---|---|---|---|---|---|---|---|---|---|
| IV administration | X | X | X | X | X | X | X | X | X | X | X | X | X |
| of study agent | | | | | | | | | | | | | |

Safety

| | | | | | | | | | | | | | | |
|---|---|---|---|---|---|---|---|---|---|---|---|---|---|---|
| Review of systems | X | X | X | X | X | X | X | X | X | X | X | X | X | X |
| Physical examination^{c} | X | | | X | | | X | | | X | | | X | |
| Body weight measurement | X | X | X | X | X | X | X | X | X | X | X | X | X | |
| Height measurement | X | X | X | X | X | X | X | X | X | X | X | X | X | |
| Vital signs | X^{d} | X^{d} | X^{d} | X^{d} | X^{d} | X^{d} | X^{d} | X^{d} | X^{d} | X^{d} | X^{d} | X^{d} | X^{d} | X |
| Routine laboratory | | | X | | | X | | | X | | | X | | X |
| analyses | | | | | | | | | | | | | | |
| ANA/Anti- | | | X | | | X | | | X | | | X | | X |
| dsDNA antibodies | | | | | | | | | | | | | | |
| QuantiFERON ®-TB | | | | | | X^{f} | | | | | | X^{f} | | |
| Gold test^{e} | | | | | | | | | | | | | | |
| TB evaluation | X | X | X | X | X | X | X | X | X | X | X | X | X | X |
| (questionnaire) | | | | | | | | | | | | | | |
| Chest x-ray^{g} | X | | | | | | | | | | | | | |
| Uveitis evaluations^{h} | | X | | | X | | | X | | | X | | | X |
| Pregnancy test (urine)ⁱ | X | X | X | X | X | X | X | X | X | X | X | X | X | |
| Infusion reaction | X | X | X | X | X | X | X | X | X | X | X | X | X | |
| evaluation^{j} | | | | | | | | | | | | | | |
| Adverse events | X | X | X | X | X | X | X | X | X | X | X | X | X | X |

Efficacy

| | | | | | | | |
|---|---|---|---|---|---|---|---|
| Joint assessments | X | X | X | X | X | X | X |
| JIA assessments^{k, l} | X | X | X | X | X | X | X |
| CRP | X | X | X | X | X | X | X |

Pharmacokinetics

| | | | |
|---|---|---|---|
| Golimumab | X | X | X |
| concentration^{m} | | | |

Immunogenicity

| | | | |
|---|---|---|---|
| Antibodies to | X | X | X |
| golimumab^{m} | | | |

^{a} All scheduled visits should occur ±1 week of the intended visit.
^{b}All subjects who discontinue study agent administration before Week 156 but do not withdraw consent must return to the study site for a final safety visit approximately 8 weeks after the last infusion (Section 10.2).
^{c}Includes skin examination at every physical examination and Tanner staging approximately every 6 months.
^{d}Vital signs should be taken pre-infusion; at 15 and 30 minutes (15-minute intervals during the infusion); and at 60 and 90 minutes (during the 1-hour observation period following the infusion).
^{e}Tuberculin skin tests should also be performed in countries where the QuantiFERON ®-TB Gold test is not approved/registered or the tuberculin skin test is mandated by local Health Authorities.
^{f}Testing is not required for subjects with a history of latent TB and ongoing treatment for latent TB or documentation of having completed adequate treatment.
^{g}Chest x-ray screening as per local and country regulations for initiation of immunosuppressive agents in children with JIA who are at risk of TB.
^{h}Evaluations (based on physical examination and interview) should be performed by the investigator at least every 6 months in all subjects. In addition, all subjects are required to have slit lamp evaluations performed by an ophthalmologist/optometrist during the study at intervals (based on JIA subtype, ANA test results, age at JIA onset, and JIA duration) as specified.
ⁱAll female subjects of childbearing potential (ie, post-menarche) must test negative for pregnancy at all visits prior to study drug administration.
^{j}Subjects will be observed for at least 60 minutes after the administration of study agent for symptoms of an infusion reaction.
^{k}JIA assessments include the following: Physician Global Assessment of Disease Activity, Childhood Health Assessment Questionnaire (CHAQ), and duration of morning stiffness. CHAQ should be completed before any tests, procedures, or other consultations for that visit to prevent influencing subjects' perceptions.
^{l} CHAQ to be completed by the parent or caregiver; preferably the same parent or caregiver should complete at every visit. Subjects who are 15 to <18 years of age at study entry may complete the assessment jointly with the parent/caregiver.
^{m}The same serum samples may be used for the measurement of golimumab concentration and detection of antibodies to golimumab. For visits with study agent administration, all blood samples for assessing golimumab concentration and antibodies to golimumab MUST be collected BEFORE the administration of the study agent.
Abbreviations: ANA = antinuclear antibodies; CHAQ = Childhood Health Assessment Questionnaire; CRP = C-reactive protein; dsDNA = double-stranded deoxyribonucleic acid; IV = intravenous; TB = tuberculosis.

TABLE 8
From Week 164 Through Week 252 (Continuation of Long-term Extension)

| | | | | | | | | | | | | |
|---|---|---|---|---|---|---|---|---|---|---|---|---|
| | | | | | | | | | | | | Final Safety |
| Week | Week | Week | Week | Week | Week | Week | Week | Week | Week | Week | Week | Follow-up |
| 164 ^{a} | 172 ^{a} | 180 ^{a} | 188 ^{a} | 196 ^{a} | 204 ^{a} | 212 ^{a} | 220 ^{a} | 228 ^{a} | 236 ^{a} | 244 ^{a} | 252 ^{a} | Visit^{b} |

Procedures and Evaluations
Administrative

| | | | | | | | | | | | | | |
|---|---|---|---|---|---|---|---|---|---|---|---|---|---|
| Concomitant | X | X | X | X | X | X | X | X | X | X | X | X | X |
| medications collection | | | | | | | | | | | | | |

Study Agent

| | | | | | | | | | | | |
|---|---|---|---|---|---|---|---|---|---|---|---|
| IV administration | X | X | X | X | X | X | X | X | X | X | X |
| of study agent | | | | | | | | | | | |

Safety

| | | | | | | | | | | | | | |
|---|---|---|---|---|---|---|---|---|---|---|---|---|---|
| Review of systems | X | X | X | X | X | X | X | X | X | X | X | X | X |
| Physical examination^{c} | | | X | | | X | | | X | | | X | |
| Body weight measurement | X | X | X | X | X | X | X | X | X | X | X | | |
| Height measurement | X | X | X | X | X | X | X | X | X | X | X | | |
| Vital signs | X^{d} | X^{d} | X^{d} | X^{d} | X^{d} | X^{d} | X^{d} | X^{d} | X^{d} | X^{d} | X^{d} | X | X |
| Routine laboratory | | X | | | X | | | X | | | X | | X |
| analyses | | | | | | | | | | | | | |
| ANA/Anti- | | X | | | X | | | X | | | X | | |
| dsDNA antibodies | | | | | | | | | | | | | |
| QuantiFERON ®-TB | | | | | X^{f} | | | | | | X^{f} | | |
| Gold test^{e} | | | | | | | | | | | | | |
| TB evaluation | X | X | X | X | X | X | X | X | X | X | X | X | X |
| (questionnaire) | | | | | | | | | | | | | |
| Chest x-ray^{g} | X | | | | | | | | | | | | |
| Uveitis evaluations^{h} | X | | | X | | | X | | | X | | X | X |
| Pregnancy test (urine)ⁱ | X | X | X | X | X | X | X | X | X | X | X | | |
| Infusion reaction | X | X | X | X | X | X | X | X | X | X | X | | |
| evaluation^{j} | | | | | | | | | | | | | |
| Adverse events | X | X | X | X | X | X | X | X | X | X | X | X | X |

Efficacy

| | | | | | | | |
|---|---|---|---|---|---|---|---|
| Joint assessments | X | X | X | X | X | X | X |
| JIA assessments^{k, l} | X | X | X | X | X | X | X |
| CRP | X | X | X | X | X | X | X |

Pharmacokinetics

| | | | |
|---|---|---|---|
| Golimumab | X | X | X |
| concentration^{m} | | | |

Immunogenicity

| | | | |
|---|---|---|---|
| Antibodies to | X | X | X |
| golimumab^{m} | | | |

^{a} All scheduled visits should occur ±1 week of the intended visit.
^{b}All subjects who discontinue study agent administration before Week 244 but do not withdraw consent must return to the study site for a final safety visit approximately 8 weeks after the last infusion (Section 10.2).
^{c}Includes skin exam and Tanner staging.
^{d}Vital signs should be taken pre-infusion; at 15 and 30 minutes (15-minute intervals during the infusion); and at 60 and 90 minutes (during the 1-hour observation period following the infusion).
^{e}Tuberculin skin tests should also be performed in countries where the QuantiFERON ®-TB Gold test is not approved/registered or the tuberculin skin test is mandated by local Health Authorities.
^{f}Testing is not required for subjects with a history of latent TB and ongoing treatment for latent TB or documentation of having completed adequate treatment.
^{g}Chest x-ray screening as per local and country regulations for initiation of immunosuppressive agents in children with JIA who are at risk of TB.
^{h}Evaluations (based on physical examination and interview) should be performed by the investigator at least every 6 months in all subjects. In addition, all subjects are required to have slit lamp evaluations performed by an ophthalmologist/optometrist during the study at intervals (based on JIA subtype, ANA test results, age at JIA onset, and JIA duration) as specified.
ⁱAll female subjects of childbearing potential (ie, post-menarche) must test negative for pregnancy at all visits prior to study drug administration.
^{j}Subjects will be observed for at least 60 minutes after the administration of study agent for symptoms of an infusion reaction.
^{k}JIA assessments include the following: Physician Global Assessment of Disease Activity, Childhood Health Assessment Questionnaire (CHAQ), and duration of morning stiffness. CHAQ should be completed before any tests, procedures, or other consultations for that visit to prevent influencing subjects' perceptions.
^{l} CHAQ to be completed by the parent or caregiver; preferably the same parent or caregiver should complete at every visit. Subjects who are 15 to <18 years of age at study entry may complete the assessment jointly with the parent/caregiver.
^{m}The same serum samples may be used for the measurement of golimumab concentration and detection of antibodies to golimumab. For visits with study agent administration, all blood samples for assessing golimumab concentration and antibodies to golimumab MUST be collected BEFORE the administration of the study agent.
Abbreviations: ANA = antinuclear antibodies; CHAQ = Childhood Health Assessment Questionnaire; CRP = C-reactive protein; dsDNA = double-stranded deoxyribonucleic acid; IV = intravenous; TB = tuberculosis.

### ABBREVIATIONS

ACR American College of Rheumatology

AE adverse event

ALT alanine aminotransferase

ANA antinuclear antibodies

ARC Anticipated Event Review Committee

AS ankylosing spondylitis

AST aspartate aminotransferase

BCG Bacille Calmette-Guérin

β-hCG n-human chorionic gonadotropin

BSA body surface area

CHAQ Childhood Health Assessment Questionnaire

CL/BSA body surface area-normalized drug clearance

CL/F apparent total systemic clearance

CRF case report form

CRP C-reactive protein

DAS Disease Activity Index Score

DMARD disease-modifying anti-rheumatic drug

DNA deoxyribonucleic acid

DRC Data Review Committee

dsDNA double-stranded deoxyribonucleic acid

eDC electronic data capture

FDA Food and Drug Administration

GCP Good Clinical Practice

HAQ Health Assessment Questionnaire

HAQ-DI Health Assessment Questionnaire Disability Index

HBsAg HBV surface antigen

HBV hepatitis B virus

HIV human immunodeficiency virus

HLA-B27 human leukocyte antigen B27

HLA-DR4 human leukocyte antigen DR4

HLA-DR5 human leukocyte antigen DR5

HLA-DR8 human leukocyte antigen DR8

ICH International Conference on Harmonisation

IEC Independent Ethics Committee

IL-1β Interleukin-1 beta

IL-6 interleukin-6

IRB Institutional Review Board

JADAS Juvenile Arthritis Disease Activity Score

JIA juvenile idiopathic arthritis

LFT liver function test

LTE long-term extension

mAb monoclonal antibody

MedDRA Medical Dictionary for Regulatory Activities

MTX methotrexate

NSAID non-steroidal anti-inflammatory drug

PD pharmacodynamic(s)

PED pediatric

pJIA polyarticular juvenile idiopathic arthritis

PK pharmacokinetic

PQC Product Quality Complaint

PPD purified protein derivative

PRCSG The Pediatric Rheumatology Collaborative Study Group

PRINTO Pediatric Rheumatology INternational Trials Organisation

PRO patient-reported outcome(s)

PsA psoriatic arthritis

q4w every 4 weeks

q8w every 8 weeks

RA rheumatoid arthritis

RBC red blood cell

RF rheumatoid factor

SAE serious adverse event

SC subcutaneous

SF-36 36-item short form health survey

SI International System of Units

SOC system organ class

TB tuberculosis

TNFα tumor necrosis factor alpha

URTI upper respiratory tract infection

US United States

VAS visual analog scale

vdH-S van der Heijde Modified Sharp

V/F apparent volume of distribution

V_{SS} volume of distribution at steady-state

WBC white blood cell

### 1. Introduction

SIMPONI® (golimumab) is a fully human monoclonal antibody (mAb) with an immunoglobulin G1 heavy chain isotype (G1m[z] allotype) and a kappa light chain isotype. The molecular weight of golimumab ranges from 149,802 to 151,064 daltons. Golimumab has a heavy chain (HC) comprising SEQ ID NO:36 and a light chain (LC) comprising SEQ ID NO:37. The molecular weight of golimumab ranges from 149,802 to 151,064 Daltons.

Golimumab forms high affinity, stable complexes with both the soluble and transmembrane bioactive forms of human tumor necrosis factor alpha (TNFα) with high affinity and specificity which prevents the binding of TNFα to its receptors and neutralizes TNFα bioactivity. No binding to other TNFα superfamily ligands was observed; in particular, golimumab does not bind or neutralize human lymphotoxin. TNFα is synthesized primarily by activated monocytes, macrophages and T cells as a transmembrane protein that self-associates to form a bioactive homotrimer that is rapidly released from the cell surface by proteolysis. The binding of TNFα to either the p55 or p75 TNF receptors leads to clustering of the receptor cytoplasmic domains and initiates signaling. Tumor necrosis factor a has been identified as a key sentinel cytokine that is produced in response to various stimuli and subsequently promotes the inflammatory response through activation of the caspase-dependent apoptosis pathway and the transcription factors nuclear factor (NF)-κB and activator protein-1 (AP-1). Tumor necrosis factor a also modulates the immune response through its role in the organization of immune cells in germinal centers. Elevated expression of TNFα has been linked to chronic inflammatory diseases such as rheumatoid arthritis (RA), as well as spondyloarthropathies such as psoriatic arthritis (PsA) and ankylosing spondylitis (AS). TNFα is an important mediator of the articular inflammation and structural damage that are characteristic of these diseases.

Blocking TNFα activity, as demonstrated in clinical studies of anti-TNFα agents, can prevent the deleterious effects caused by excessive TNFα. SIMPONI® (golimumab) for intravenous (IV) use is being developed to offer an alternative route of administration (compared with other available anti-TNFα agents) and a convenient dose regimen (ie, every 8 week [q8w] administration) for patients with polyarticular JIA (pJIA).

### 1.1. Background

### 1.1.1. Juvenile Idiopathic Arthritis

Juvenile idiopathic arthritis is a diagnosis of exclusion that encompasses all forms of arthritis that begin before the age of 16 years, persist for more than 6 weeks and are of unknown cause.¹⁸ It is the most common chronic rheumatic disease in children and is categorized according to the International League of Associations for Rheumatology (ILAR) classification into 7 subtypes (systemic arthritis, oligoarthritis, rheumatoid factor [RF]-negative polyarthritis, RF-positive polyarthritis, enthesitis-related arthritis, psoriatic arthritis, undifferentiated arthritis) characterized by distinct clinical presentations and features.¹⁶

The heterogeneity of JIA indicates that multiple factors contribute to the etiology and pathogenesis of the disease, and both genetic and environmental factors have been implicated. These include implicating infection as a triggering mechanism, links between human leukocyte antigen (HLA) and non-HLA molecules and disease development, and immunological abnormalities leading to tissue inflammation and joint destruction. The role of infection in disease development is still unproven.¹⁸ However, in JIA, HLA-DR5 and HLA-DR8 locus antigens have been implicated as associated contributory elements in young girls with oligoarticular arthritis, whereas HLA-DR4 has been implicated in RF-positive polyarticular arthritis in older children, and HLA-B27 has been implicated in older boys with oligoarticular disease.^{15,17}

Although the etiology and pathogenesis of JIA are still unclear, the same cell types and underlying mechanisms that play a role in the progression of adult RA are probably involved.¹⁵ The cellular entities involved include macrophages that elaborate a number of inflammatory cytokines and mediators of inflammation. Macrophage-derived cytokines, such as TNFα, appear to play a critically important role in the induction and perpetuation of chronic inflammatory processes in the joints of patients with RA as well as in the systemic manifestations of this disease,⁶ though the role of TNFα in systemic JIA is less convincing.³

Some studies have shown that levels of inflammatory cytokines (eg, interleukin-1 beta [IL-1β, interleukin-6 [IL-6], and TNFα) elevated in adults with RA are also elevated in the synovial fluid and serum of patients with JIA.^{9,19,12,3,20} These studies have also found different cytokine profiles among patients with various JIA subgroups.

Juvenile idiopathic arthritis is an important cause of short-term and long-term disability in children,¹⁴ but new advances in therapy have demonstrated clinically important steps forward. In the past 10 years, studies have shown that 40% to 60% of patients have inactive disease or clinical remission while on medication for JIA at follow-up. Functional outcome has improved in the last decade, with 2.5% to 10% of patients with serious functional disability.¹⁸ However, particularly serious complications of JIA include linear growth suppression, osteoporosis, local growth disturbances, macrophage activation syndrome and iridocyclitis.¹⁸

The aim of treatment in JIA is to obtain complete control of the disease, to preserve the physical and psychological integrity of the child and to prevent any long-term consequence related to the disease or its therapy. The mainstays of treatment in JIA have been NSAIDs, intra-articular and systemic corticosteroids, methotrexate (MTX), and other DMARDs. The introduction of biological medications has provided an important new therapeutic option for the treatment of patients with JIA who are resistant to conventional anti-rheumatic agents.¹⁸ Currently approved biologic therapies for the treatment of pJIA include etanercept, adalimumab, abatacept, and tocilizumab; canakinumab and tocilizumab have been approved for systemic JIA.

### 1.1.2. Golimumab Clinical Studies in Rheumatoid Arthritis and Juvenile Idiopathic Arthritis

Golimumab given as a SC injection has been demonstrated to be efficacious in adults with RA, PsA, ankylosing spondylitis (AS), and ulcerative colitis. Intravenous golimumab has also proven effective in adults with RA. Other anti-TNFα agents have been effective in the treatment of subjects with JIA. The Sponsor conducted a study of BSA-based dosages of SC golimumab (CNTO148JIA3001) to assess the benefits and risks associated with the use of SC golimumab in the treatment of multiple subtypes of JIA, including juvenile PsA.

The results of the CNTO148ART3001 study of IV golimumab in adults and the results of the CNTO148JIA3001 study of SC golimumab in subjects with JIA are described below.

### 1.1.2.1. Intravenous Golimumab in Adult Rheumatoid Arthritis

The primary objective of CNTO148ART3001, a randomized, placebo-controlled, multicenter, double-blind study, was to assess the clinical efficacy of IV administration of golimumab 2 mg/kg+MTX compared with MTX alone in adult subjects with active RA despite MTX therapy. Approximately 564 subjects were planned, and 592 were randomized.

Subjects were men or women 18 years of age or older with a diagnosis of RA for at least 3 months prior to screening who had active RA, defined as ≥6 tender and ≥6 swollen joints, at screening and at baseline, despite concurrent MTX therapy. At screening, subjects had to have C-reactive protein (CRP) measurement of ≥1.0 mg/dL (upper limit of normal=1.0 mg/dL) and be RF-positive.

Subjects randomized to golimumab received 2 mg/kg of golimumab intravenously over a 30±10 minute infusion time. Additionally, subjects were maintained on their stable dose of commercial MTX (between 15 mg and 25 mg/week) throughout the study.

Randomization was stratified based upon a screening CRP of <1.5 mg/dL or ≥1.5 mg/dL. Subjects were randomized 2:1 to golimumab+MTX or placebo+MTX at Week 0, Week 4, and every 8 weeks (q8w) thereafter. The duration of treatment for the entire study was 100 weeks with a 12 week safety follow-up period.

In total, 570 (96%) of 592 subjects completed the 24-week study. The remaining 22 (4%) subjects discontinued the study before Week 24. Most discontinuations were due to AEs: 9 [2.3%] subjects in the golimumab+MTX group and 2 [1.0%] subjects in the placebo+MTX group).

A significantly greater proportion of subjects in the golimumab+MTX group (58.5%) achieved the primary endpoint, an ACR 20 response at Week 14, compared with subjects in the placebo+MTX group (24.9%, p<0.001). The treatment effect was consistent in subjects with either a CRP≥1.5 mg/dL or <1.5 mg/dL at screening. A significant difference in the proportion of ACR 20 responders between the golimumab+MTX and placebo+MTX groups was observed as early as Week 2. Major secondary efficacy endpoints were also achieved. A significantly greater proportion of subjects in the golimumab+MTX group had good or moderate Disease Activity Index Score (DAS)28 responses (using CRP) at Week 14 (81.3%) compared with subjects in the placebo+MTX group (40.1%, p<0.001).

There was a significantly greater improvement in Health Assessment Questionnaire Disability Index (HAQ-DI) disability scores at Week 14 in subjects in the golimumab+MTX group (0.500) compared with subjects in the placebo+MTX group (0.125, p<0.001). There was also a significant difference in clinically relevant improvements in HAQ-DI (≥0.25) in the golimumab+MTX group compared with the placebo+MTX group both at Week 14 (68.4% compared with 43.1%, respectively, p<0.001) and at Week 24 (67.6% compared with 45.2%, respectively, p<0.001). Subjects who received golimumab+MTX demonstrated significantly greater ACR 50 response rates at Week 24 (34.9%) compared with subjects who received placebo+MTX (13.2%, p<0.001).

A consistent treatment benefit was observed within subgroups of demography, baseline clinical characteristics, and prior exposure to medications for RA except for subgroups with small population size (ie, <15 subjects).

Statistically significant greater improvement in the mental and physical component summary scores of the 36-item short form health survey (SF-36) as well as all 8 scales of the SF-36 instrument were observed in golimumab+MTX treatment relative to placebo+MTX treatment at Week 12 (p<0.001 for all comparisons). These improvements were maintained through Week 24.

Through Week 16 (the placebo-controlled period prior to early escape) in CNTO148ART3001, 43.7% of subjects in the placebo group and 47.3% in the golimumab group had an AE; the highest incidence of AEs was in the Infections and infestations system organ class (SOC), 20.8% and 24.3% in the placebo and golimumab groups, respectively, with upper respiratory tract infection (URTI) being the most frequently reported AE (5.6% and 5.1% in the placebo and golimumab groups, respectively. Through Week 112, 79.1% of golimumab-treated subjects had an AE; the highest incidence of AEs was in the infections and infestations SOC (50.5%) and URTI was the most frequently reported AE (11.5%).

Through Week 16 in CNTO148ART3001, 1.0% of subjects in the placebo group and 3.8% of subjects in the golimumab group had an SAE. The incidence of SAEs within each SOC was <1.0%, and no SAE occurred in more than 1 subject. Through Week 112, 18.2% of golimumab-treated subjects had an SAE; the highest incidence of SAEs occurred in the infections and infestations SOC (5.5%) and musculoskeletal and connective tissue disorders SOC (3.4%) and the most frequently reported SAE was RA (2.1%).

Through Week 24, 1 patient died in the CNTO148ART3001 study; this subject was randomized to treatment with placebo+MTX, had never received golimumab, and died of a presumed cerebrovascular accident (stroke). Through Week 112, an additional 5 subjects died in the CNTO148ART3001 study. Two subjects randomized to treatment with placebo+MTX died, both after switching to golimumab 2 mg/kg+MTX; cause of death was sudden death (n=1) and complications of severe dehydration, Clostridium difficile colitis, and atrial fibrillation (n=1). Three subjects randomized to treatment with 2 mg/kg golimumab+MTX died in the study; reported cause of death was acute abdominal syndrome (later diagnosed as peritoneal tuberculosis [TB], n=1), presumed myocardial infarction (MI, n=1), and septic shock secondary to a pyogenic lung abscess due to Acinetobacter baumannii (n=1).

No malignancies were reported through Week 16 in study CNTO148ART3001. There was 1 case of nontreatment-emergent lung adenocarcinoma reported in the placebo+MTX group prior to receiving study agent. Through the placebo-controlled period (Week 24), 1 malignancy (breast cancer) was reported in the golimumab group. Through Week 112, 5 additional malignancies were reported, including basal cell carcinoma, chronic lymphocytic leukemia in a subject with a family history of chronic lymphocytic leukemia, cervix carcinoma in situ, Bowen's Disease, and basal cell carcinoma. No lymphomas were reported through Week 112.

Through Week 16 in CNTO148ART3001, 0.8% of subjects in the golimumab group had a serious infection, including appendicitis, bacteremia, and (complications of) interstitial lung disease. No subjects in the placebo group had a serious infection. Through Week 112, 6.2% of golimumab-treated subjects had a serious infection. Serious infections occurring in more than one subject were pneumonia (n=5), UTI (n=4), and erysipelas (n=2).

Through Week 16 in CNTO148ART3001, 0.5% of subjects in the placebo group and 2.5% of subjects in the golimumab group had an infusion reaction. Through Week 112, 3.9% of golimumab-treated subjects had an infusion reaction and 0.4% of infusions were complicated by infusion reactions. It should be noted that all placebo infusions consisted of 0.9% normal saline alone rather than a true matched placebo. No serious infusion reactions requiring study agent discontinuation were noted. There was a case of anaphylaxis, which was not associated with study drug.

The median peak serum golimumab concentration (ie, post-infusion golimumab concentration) of 41.56 μg/mL was observed at Week 4 following IV administration of 2 mg/kg golimumab at Week 0, Week 4, followed by q8w (±1 week) administration. This peak is higher than that reported for SC golimumab administration of 50 mg every 4 weeks (q4w). The median trough serum golimumab concentration in subjects receiving IV golimumab at 2 mg/kg q8w with MTX was 0.28 μg/mL at Week 12 and 0.22 μg/mL at Week 20; these levels are similar to those reported with SC golimumab 50 mg. Overall exposure to golimumab is approximately 3 times that for SC golimumab 50 mg over a similar period of exposure.

Data from the IV golimumab program demonstrated less radiographic progression in subjects treated with golimumab compared with subjects who received placebo. There was a significant difference in change from baseline in total van der Heijde Modified Sharp (vdH-S) score at Week 24 (placebo+MTX: 1.09±3.194, golimumab 2 mg/kg+MTX: 0.03±1.899 [p<0.001]) between the golimumab+MTX treatment group and placebo+MTX. Significant differences in favor of IV golimumab were also observed in changes from baseline in erosion and joint space narrowing scores. The proportion of subjects with radiographic progression based on the smallest detectable change was significantly lower for subjects treated with golimumab+MTX when compared with placebo+MTX for the total vdH-S score (p<0.001) as well as both erosion (p=0.001) and joint space narrowing measurements (p=0.01).

### 1.1.2.2. Subcutaneous Golimumab in Juvenile Idiopathic Arthritis

CNTO148JIA3001 was a randomized withdrawal, double-blind, placebo-controlled, parallel-group, multicenter study of BSA-based 30 mg/m² (up to a maximum 50 mg/dose) SC golimumab given every 4 weeks (q4w) in pediatric subjects with active pJIA despite current treatment with MTX. The study population comprised subjects with pJIA receiving MTX, ages 2 to less than 18 years, with at least a 6-month history of pJIA, and active arthritis in ≥5 joints. All subjects received SC golimumab in the active treatment portion of the study from Week 0 through Week 16. At Week 16, JIA ACR 30 responders were randomized to receive placebo or golimumab for 32 weeks; subjects randomized to placebo who experienced flares during this 32-week period had golimumab therapy re-instituted. The placebo-controlled period was through Week 48, and the long-term extension was planned from Week 48 through Week 248.

Approximately 170 subjects were planned, and 173 subjects were enrolled into the study. All of the 173 subjects were included in the Week 48 efficacy and safety analyses. Nineteen of the 173 subjects discontinued study agent through Week 16 (due to: lack of efficacy [n=14]; AEs [n=4]; withdrawal of consent [n=1]), and 154 subjects entered randomized withdrawal (76 to placebo and 78 continued golimumab).

The baseline disease characteristics of the 173 enrolled subjects constituted a population with moderate to severe JIA comparable with other clinical studies of anti-TNFα agents in pJIA, with the exception of numerically lower mean and median CRP/ESR levels in CNTO148JIA3001.

The proportion of subjects who were JIA ACR 30 responders at Week 16 was 87.3%. Additionally, the proportion of JIA ACR 50, JIA ACR 70, and JIA ACR 90 responders at Week 16 were 79.2%, 65.9%, and 36.4%, respectively.

The study did not meet its primary and major secondary endpoints as the proportion of subjects who were JIA ACR 30 responders at Week 16 and did not experience a flare of disease between Week 16 and Week 48 was not significantly different in subjects randomized to continued golimumab treatment between Weeks 16 and 48 as compared with subjects randomized to receive placebo between Weeks 16 and 48 (59% versus 52.6%, p=0.41). All sensitivity analyses and major secondary endpoints demonstrated the lack of statistically significant differences between treatment groups. The Sponsor terminated the long-term extension of the study early as pre-specified efficacy endpoints were not met.

Post-hoc analyses that evaluated flare rates based on Week 0 CRP levels ranging from 0.1-1.0 mg/dL demonstrated that, in general, among subjects with higher baseline CRP levels, the subjects who received continued golimumab therapy had significantly fewer flare episodes than subjects who were randomized to placebo at Week 16.

When JIA ACR response rates were analyzed based on observed data through Week 48 (using Week 0 as baseline and comparing drug/placebo effect at each visit through Week 48), JIA ACR 30 response rates of 89% to 95.9% and JIA ACR 90 response rates of 53.4% to 56.2% were achieved at Week 48. Improvements in the core sets through Week 48 were similar at all visits in subjects randomized to golimumab at Week 16 as compared with subjects randomized to placebo at Week 16 and all represented clinically meaningful improvement in disease, eg, median percent improvement of 94.6% and 95.1% in Physician Global Assessment of Disease Activity, and median percent improvement of 90.9% and 100% in the number of active joints.

Pharmacokinetic (PK) and immunogenicity data were collected through Week 48 in CNTO148JIA3001. In subjects with pJIA who received golimumab 30 mg/m² SC and were randomized to stay on active treatment, median trough golimumab concentrations at Week 12, Week 24, and Week 48 were 1.16 μg/mL, 1.12 μg/mL, and 0.95 μg/mL, respectively, indicating that steady-state levels were maintained though Week 48. Furthermore, steady-state trough golimumab concentrations were similar across different age groups, body weight quartiles, body mass index quartiles, and body weight categories in subjects with pJIA. Overall, these concentrations were similar to the PK exposure observed in the adult active RA population (despite MTX) in C0524T06 treated with SC golimumab, and thus supported the hypothesis that the BSA-based golimumab regimen of 30 mg/m² SC q4w was sufficient to produce concentrations comparable to that seen in the adult RA population who received golimumab 50 mg SC q4w. Further, PK and efficacy analyses showed that similar efficacy (as measured by JIA ACR 30 response, and flare rates) were seen in subjects with pJIA in the 4 subgroups of steady-state trough golimumab concentration quartiles. Additionally, there were no apparent PK differences observed between subjects with and without flares.

With regards to immunogenicity, 40.1% of subjects developed antibodies to golimumab using the recently developed drug tolerant immunoassay analyses. The new drug tolerant immunoassay is more sensitive compared with assays used previously in adult golimumab RA studies and allows the detection of antibodies to golimumab despite detectable serum golimumab levels. Among subjects who were randomized and remained on golimumab 30 mg/m² SC+MTX, 30.8% developed antibodies to golimumab; antibody titers tended to be low. When evaluating the effects of immunogenicity on PK, efficacy, and safety, it was found that positive anti-golimumab antibody status decreased steady-state trough golimumab concentrations when the titer levels were >1:100. However, the effect of antibodies on efficacy was less sensitive, requiring higher titers >1:1000 in order to correlate with apparent reductions in efficacy. Since only approximately 5% of subjects with JIA developed anti-golimumab antibodies with titers >1:1000, it was determined that immunogenicity was not a contributing factor to lack of achievement of the primary endpoint in CNT0148JIA3001. Additionally, positive anti-golimumab antibody status did not appear to be associated with a higher incidence of injection-site reactions.

The proportion of subjects who reported an AE through Week 48 was 87.9%. The most commonly reported system organ class of AEs was Infections and infestations (67.1%), and were predominantly upper respiratory tract infections and nasopharyngitis. There was no marked difference in AEs reported between Week 16 and Week 48 for subjects randomized to placebo (82.9%) and those randomized to continued golimumab treatment (78.2%); however, it needs to be noted that all subjects in randomized withdrawal portion of the study were exposed to golimumab for 16 weeks before re-randomization. Serious adverse events were reported by 13.3% of subjects. The most commonly reported SAE was worsening of JIA (6.4%). Serious infections were reported in 2.9% of subjects (pneumonia, urinary tract infection, herpes zoster, upper respiratory tract infection, and pyelonephritis), and there were no deaths, malignancies, or demyelination events through Week 48. There were no reports of active TB and no serious opportunistic infections. Through Week 48, the number of subjects with abnormal alanine aminotransferase (ALT) measurements (and no concomitant treatment for latent TB, which may affect liver function tests [LFTs]) was 29.5% (51/167), 39 of the 51 subjects had elevations <3× ULN.

There were 2 subjects with ALT elevation to >8× ULN but neither subject met the criteria for Hy's Law consistent with hepatotoxicity. Subjects were not receiving TB prophylaxis; one of the subjects had baseline ALT which was already abnormal. All subjects with LFT abnormalities were managed conservatively with changes in MTX dosing but one subject was discontinued for elevated LFTs.

The incidence of injections with injection-site reactions through Week 48 was 0.8%; there was one SAE report of serum sickness-like reaction in a subject randomized to placebo who resumed golimumab treatment.

Although the CNTO148JIA3001 study did not meet its endpoints, when JIA ACR response rates were analyzed as observed data through Week 48 (using Week 0 as baseline and comparing drug/placebo effect at each visit through Week 48) the study showed the potential for efficacy that could be attained with SC golimumab in children with pJIA. Therefore, it lends support to the study of IV golimumab in subjects with pJIA who have an inadequate response to MTX.

### 1.2. Overall Rationale for the Study

Intravenous golimumab has been demonstrated to be efficacious in the treatment of adults with RA (Section 1.1.2.1). Other biologics, including anti-TNFα agents, have been shown to be effective in the treatment of subjects with pJIA. Though biologic infusion therapies are available for the treatment of pJIA, there are currently no approved intravenously administered anti-TNFα agents for this condition. The every 8 week, 30-minute infusion paradigm proposed in this study for children and studied in adults with RA may be appropriate for populations of patients where greater physician scrutiny of drug therapy may be needed or requested. Particularly in the pediatric population, the reduction in the number of drug administrations (ie, to an every 8 week maintenance schedule) could provide greater convenience and less pain (due to fewer IV administrations) compared with other biologic agents. In addition, switching to a different anti-TNFα agent in a patient in whom a previous anti-TNFα agent was not efficacious may provide further symptomatic relief of disease.

The primary objective of this study is to characterize the PK of IV golimumab in pJIA, along with evaluations of the safety and efficacy of IV golimumab in these subjects. This study will also include subjects with multiple subtypes of JIA, including juvenile PsA, as well as subjects with prior anti-TNFα experience (up to 30% of the study population).

The study is designed to obtain PK data in response to BSA-based (80 mg/m², which is expected to be equivalent to the 2 mg/kg dose in adult RA patients weighing 70 kg) IV golimumab for subjects with pJIA who have inadequate response to MTX treatment as well as prior treatment with non-steroidal anti-inflammatory agents, corticosteroids, and/or anti-TNFα agents, with the intent to demonstrate its similarity to the response seen with weight-based (2 mg/kg) doses of IV golimumab in adult RA subjects who have inadequate response to MTX treatment. The 80 mg/m² dose for subjects with pJIA is based on the 2 mg/kg dose studied in CNTO148ART3001 in the adult RA population.

### 2. Objectives and Hypothesis

### 2.1. Objectives

### Primary Objective

The primary objective of this study is to assess the PK following intravenously administered golimumab in subjects (ages 2 to less than 18 years) with pJIA manifested by ≥5 joints with active arthritis despite MTX therapy for ≥2 months.

### Secondary Objectives

The secondary objectives of this study are to evaluate IV golimumab in subjects with pJIA with respect to PK, efficacy (relief of signs and symptoms, physical function, and quality of life), safety (AEs, SAES, and assessment of laboratory parameters), and immunogenicity (antibodies to golimumab).

### 2.2. Hypothesis

No formal hypothesis testing is planned in this study.

### 3. Study Design and Rationale

### 3.1. Overview of Study Design

This is a Phase 3, open-label, single-arm, multicenter study to evaluate the PK, safety, and efficacy of IV golimumab in subjects with active pJIA despite current treatment with MTX. The study population will comprise subjects with pJIA receiving MTX, ages 2 to less than 18 years, with at least a 3-month history of pJIA, and active arthritis in ≥5 joints. Approximately 120 subjects will be enrolled at Week 0 to ensure that approximately 100 subjects remain in the study at Week 52. Enrollment patterns are expected to yield a subject population of approximately 10% aged 2 to up to 6 years, approximately 20% aged 6 to up to 12 years, and approximately 70% aged 12 to less than 18 years.

All subjects will receive 80 mg/m² golimumab (maximum single dose 240 mg) as an IV infusion given over 30±10 minutes at Weeks 0, 4, and every 8 weeks (q8w; ±3 days) through Week 28 and then q8w (±1 week) thereafter through Week 244. Body surface area will be calculated based on the subject's height and body weight measured at each visit, and the BSA-based dose of golimumab will be adjusted as needed to maintain the dose at 80 mg/m². Subjects will also receive commercial MTX weekly through Week 28 at the same BSA-based dosage (10 to 30 mg/m² per week of MTX in subjects with BSA<1.67 m², or a minimum of 15 mg/week in subjects with BSA≥1.67 m²) as at time of study entry as outlined in Section 6.2.

Every effort should be made to maintain subjects at a dose of 80 mg/m² of golimumab based upon BSA, and decreases below or increases above 80 mg/m² or shortening of the dosing interval (eg, from 8 weeks to 6 weeks) will not be permitted at any visit.

This is an open-label study, with all subjects receiving the same BSA-based dose of IV golimumab. Safety data will be routinely evaluated by the study's medical monitor. Therefore, an external Data Monitoring Committee will not be established.

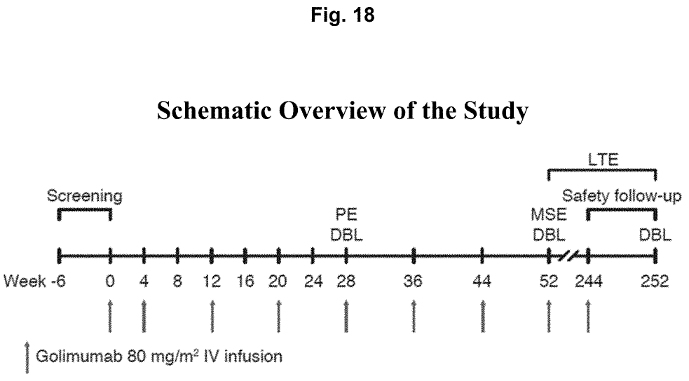

A diagram of the study design is provided in FIG. 18.

3.1.1. Week 0 through Week 28

Through Week 28, subjects will be monitored and disease activity and safety will be assessed at the investigative site every 4 weeks.

If <50% of the study population achieves an adequate response to the treatment (American College of Rheumatology Pediatric 30% [JIA ACR 30] response) at Week 28, the study will be discontinued.

After all subjects complete the Week 28 visit, the database will be locked to assess PK, safety and efficacy. An additional safety, efficacy, and PK database lock is currently planned for Week 52. Final database lock will be performed at Week 252.

No changes should be made to background medications (ie, MTX, other DMARDs, corticosteroids, and NSAIDs) in terms of increases or decreases in dosage beyond the parameters provided in Section 8 (eg, no more than 10 mg/day prednisone or no more than 0.20 mg/kg/day, whichever is lower) and/or route of administration between Weeks 0 and 28, unless there is a safety concern (eg, elevated liver function tests), which requires changes to background medications.

If a subject is lost to follow-up, every possible effort must be made by the study site personnel to contact the subject and determine the reason for discontinuation/withdrawal. The measures taken to follow-up must be documented.

When a subject withdraws before completing the study, the reason for withdrawal is to be documented in the CRF and in the source document. Study drug assigned to the withdrawn subject may not be assigned to another subject. Subjects who withdraw will not be replaced.

3.1.2. Week 28 through Week 52

From Week 28 through Week 52, infusions will continue to be performed every 8 weeks (±1 week); however, subjects will be actively monitored at the investigative site and disease activity and safety will be assessed at the investigative site every 8 weeks rather than every 4 weeks as between Weeks 0 and 28. As noted above, after Week 28 subjects will be permitted to change/add MTX, other DMARDs, corticosteroids, and NSAIDs as outlined in Section 8.

3.1.3. Week 52 through Week 252 (Long-Term Extension)

Subjects who complete the study at Week 52 will have the option to enter into the long-term extension (LTE) phase of this study. Subjects who opt not to enter the long-term extension will be encouraged to complete an additional 8-week safety follow-up visit following the last administration of study agent.

During the long-term extension, all subjects will continue to receive golimumab q8w (±1 week) through Week 244. For children who have completed the full trial period of 252 weeks and for whom drug is proven beneficial but is not commercially available for pJIA indication (or patient does not qualify for insurance to pay for the drug) IV golimumab will continue to be provided by the Sponsor. Between Week 52 and Week 252, disease activity will be monitored and assessed, and documented in the CRF every 16 weeks; infusions and safety measurements will be done every 8 weeks at the investigative site.

As noted above, after Week 28, subjects will be permitted to change/add MTX, other DMARDs, corticosteroids, and NSAIDs, including increases or decreases in BSA-based dosing (where appropriate) for these classes of agents as outlined in Section 8.

All subjects who complete the Week 244 visit are expected to participate in the safety follow-up visit at Week 252. Those subjects who discontinue study agent at any time before Week 244 are also expected to return for a safety follow-up visit approximately 8 weeks after the last administration of study agent.

The final database lock will be at Week 252.

### 3.1.4. End of Study Definition

The end of the study is defined as the last follow-up assessment for the last subject in the long-term extension.

### 3.2. Study Design Rationale

### 3.2.1. Blinding, Control, Study Phase/Periods, Treatment Groups

This is a single-arm, open-label study to evaluate the PK of IV golimumab in subjects with pJIA, with all subjects receiving the same BSA-based dose of IV golimumab through Week 52. Subjects who complete the study at Week 52 will have the option to enter into the long-term extension phase of this study through Week 252.

### 3.2.2. Dose Selection

Unlike adult drug doses, pediatric drug doses (parenteral) are commonly calculated individually as weight-based (mg/kg) or BSA-based (mg/m²) doses to manage the PK variability observed in children across different ages as changes occur in their maturing organ systems.^{10,22} The successful outcome of dose extrapolation from adults to pediatric subjects through weight-based or BSA-based dose normalization for other approved anti-TNFα agents (eg, adalimumab and etanercept) supports the assumption that clinical responses to anti-TNFα agents in rheumatoid disease would be similar between adults and children. That is, after the PK differences inherent between adults and children are accounted for, similar drug responses would be expected with similar drug exposure in both adults and children.

Data from the Phase 3 IV study in adults with RA (CNTO148ART3001) through 24 weeks have shown that golimumab 2 mg/kg at Week 0, Week 4, and q8w (±1 week) thereafter is the optimal dose regimen for the treatment of RA in most adults. For a child, golimumab 80 mg/m² (2 mg/kg/1.73 m²) would be approximately equivalent to 2 mg/kg for an adult subject weighing 70 kg (with a BSA of 1.73 m²). Thus, in the current study (CNT0148JIA3003), a dose of golimumab 80 mg/m² has been chosen to evaluate the safety and efficacy of golimumab in the pJIA population.

### 3.2.3. Rationale

The open-label study design for IV golimumab in the pJIA population is based on data from studies of other anti-TNFα agents in adult RA and pJIA, PK and efficacy data from the Sponsor's study of IV golimumab in adult RA (CNTO148ART3001), the Sponsor's experience with SC golimumab in pJIA (CNTO148JIA3001), and feedback from the Pediatric Rheumatology International Trials Organisation (PRINTO) and The Pediatric Rheumatology Collaborative Study Group (PRCSG).

The Sponsor will utilize PK data generated from the proposed open-label CNTO148JIA3003 study to extrapolate to adult PK data from the CNTO148ART3001 study in RA, which was the pivotal study that served as the basis for approval of IV golimumab (SIMPONI ARIA/SIMPONI for Intravenous Use) for adult patients with RA. Additionally, efficacy (PD) data will be collected to explore the assessment of supportive exposure-response.

### 4. Subject Population

Screening for eligible subjects will be performed within 6 weeks before administration of the study drug.

The inclusion and exclusion criteria for enrolling subjects in this study are described in the following 2 subsections. If there is a question about the inclusion or exclusion criteria below, the investigator should consult with the appropriate Sponsor representative before enrolling a subject in the study.

For a discussion of the statistical considerations of subject selection, refer to Section 11.2, Sample Size Determination.

Deviations from the inclusion and exclusion criteria are not allowed because they can potentially jeopardize the scientific integrity of the study, regulatory acceptability, or subject safety. Therefore, adherence to the criteria as specified in the protocol is essential.

Approximately 120 subjects will be enrolled in this study. Enrolled subjects who discontinue study treatment or withdraw from study participation will not be replaced with new subjects.

Retesting of an abnormal screening value that leads to exclusion is allowed only once using an unscheduled visit during the screening period to reassess eligibility. This should be considered only if there is no anticipated impact on subject safety.

### 4.1. Inclusion Criteria

Each potential subject must satisfy all of the following criteria to be enrolled in the study.
1. Subjects must be age 2 years to less than 18 years with a body weight >15 kg at the time of screening and at Week 0.
2. Diagnosis must be made per JIA ILAR diagnostic criteria and the onset of disease must have been before the subject's 16th birthday.
3. Active JIA of one of the following subtypes:
   a. Rheumatoid factor positive or negative pJIA for ≥3 months prior to screening, or
   b. Systemic JIA with no systemic symptoms for ≥3 months, but with polyarthritis for ≥3 months prior to screening, or
   c. Extended oligoarticular JIA≥3 months prior to screening, or
   d. Polyarticular juvenile psoriatic arthritis ≥3 months prior to screening, or,
   e. Enthesitis related arthritis ≥3 months prior to screening.
4. Failure or inadequate response to at least a 2-month course of MTX before screening.
5. Subjects must have ≥5 joints with active arthritis at screening and at Week 0 as defined by ACR criteria (ie, a joint with either swelling, or in the absence of swelling, limited range of motion associated with pain on motion or tenderness).
6. Subjects must have a screening CRP of ≥0.1 mg/dL with the exception of approximately 30% of the study population.
7. Subjects must have active pJIA despite current use of oral, intramuscular, or subcutaneous MTX for ≥2 months before screening. For subjects with BSA<1.67 m², the MTX dose must be between 10 to 30 mg/m² per week and stable for ≥4 weeks before screening. For subjects with BSA≥1.67 m², the MTX dose must be a minimum of 15 mg/week and must be stable for ≥4 weeks before screening. In situations where there is documented intolerance of doses >10 mg/m² weekly (for subjects with BSA<1.67 m²) or ≥15 mg/week (for subjects with BSA≥1.67 m²); or where documented country or site regulations prohibit use of ≥15 mg of MTX per week in subjects with BSA≥1.67 m², subjects may be entered into the trial on a lower dose of MTX.
8. If using corticosteroids, must be on a stable dose of ≤10 mg/day prednisone equivalent or 0.20 mg/kg/day (whichever is lower) for ≥2 weeks before first administration of study agent. If currently not using corticosteroids, the subject must have not received corticosteroids for at least 2 weeks before the first dose administration. Subjects with systemic onset JIA but without systemic symptoms must be on a stable dose of corticosteroids for at least 3 days prior to screening.
9. If using NSAIDs, must be on a stable dose for ≥2 weeks before screening. If not currently using NSAIDs, must not have taken them for at least 2 weeks before screening.
10. Subjects are considered eligible according to the following TB screening criteria:
   a. Have no history of latent or active TB prior to screening. An exception is made for subjects currently receiving treatment for latent TB with no evidence of active TB, or who have a history of latent TB and documentation of having completed appropriate treatment for latent TB within 3 years prior to the first administration of study agent. It is the responsibility of the investigator to verify the adequacy of previous anti-tuberculous treatment and provide appropriate documentation.
   b. Have no signs or symptoms suggestive of active TB upon medical history and/or physical examination.
   c. Have had no recent close contact with a person with active TB or, if there has been such contact, will be referred to a physician specializing in TB to undergo additional evaluation and, if warranted, receive appropriate treatment for latent TB prior to the first administration of study agent.
   d. Within 6 weeks prior to the first administration of study agent, have a negative QuantiFERON® (TB Gold test) result, or have a newly identified positive QuantiFERON® (TB Gold test) result in which active TB has been ruled out and for which appropriate treatment for latent TB (Section 9.1.2) has been initiated prior to the first administration of study agent. Within 6 weeks prior to the first administration of study agent, a negative tuberculin skin test, or a newly identified positive tuberculin skin test in which active TB has been ruled out and for which appropriate treatment for latent TB has been initiated prior to the first administration of study agent, is additionally required if the QuantiFERON® (TB Gold test) is not approved/registered in that country or the tuberculin skin test is mandated by local Health Authorities.
   e. Indeterminate results should be handled as outlined in Section 9.1.2. Subjects with persistently indeterminate QuantiFERON® (TB Gold test) results may be enrolled without treatment for latent TB, if active TB is ruled out, their chest radiograph shows no abnormality suggestive of TB (active or old, inactive TB), and the subject has no additional risk factors for TB as determined by the investigator. This determination must be promptly reported to the Sponsor's medical monitor and recorded in the subject's source documents and initialed by the investigator.
   f. The QuantiFERON® (TB Gold test) and the tuberculin skin test are not required at screening for subjects with a history of latent TB and ongoing treatment for latent TB or documentation of having completed adequate treatment as described above; Subjects with documentation of having completed adequate treatment as described above are not required to initiate additional treatment for latent TB.
   g. Unless country or local guidelines do not recommend a chest radiograph as a necessary screening process prior to initiation of anti-TNFα therapies, a chest radiograph (posterior-anterior view) must have been taken within 3 months prior to the first administration of study agent and read by a qualified radiologist, with no evidence of current active TB or old inactive TB. Chest radiographs (both posterior-anterior and lateral views) must be performed as part of the screening process in all cases when either the tuberculin skin test and/or QuantiFERON® (TB Gold test) for TB is positive.
11. Subjects must be medically stable on the basis of physical examination, medical history, and vital signs performed at screening. If there are abnormalities, they must be consistent with the underlying illness in the study population.
12. Girls must be either:
   Not of childbearing potential: premenarchal; permanently sterilized (eg, tubal occlusion, hysterectomy, bilateral salpingectomy); or otherwise be incapable of pregnancy,
   OR
   Of childbearing potential, and if sexually active, practicing a highly effective method of birth control consistent with local regulations regarding the use of birth control methods for subjects participating in clinical studies: eg, established use of oral, injected or implanted hormonal methods of contraception; placement of an intrauterine device (IUD) or intrauterine system (IUS); barrier methods: condom with spermicidal foam/gel/film/cream/suppository or occlusive cap (diaphragm or cervical/vault caps) with spermicidal foam/gel/film/cream/suppository; male partner sterilization (the vasectomized partner should be the sole partner for that subject); true abstinence (when this is in line with the preferred and usual lifestyle of the subject and at the discretion of the investigator/per local regulations). Girls of childbearing potential must agree not to donate eggs (ova, oocytes) for the purposes of assisted reproduction during the study and for 6 months after receiving the last dose of study drug. Note: If the childbearing potential changes after start of the study (eg, girl who is not heterosexually active becomes active, premenarchal girl experiences menarche) a girl must begin a highly effective method of birth control, as described above.
13. Girls of childbearing potential must have a negative serum n-human chorionic gonadotropin (β-hCG) test at screening and a negative urine pregnancy test at each study visit where golimumab infusion is to take place.
14. Boys must practice abstinence, or if sexually active with a girl of childbearing potential and has not had a vasectomy must agree to use a barrier method of birth control eg, either condom with spermicidal foam/gel/film/cream/suppository or partner with occlusive cap (diaphragm or cervical/vault caps) with spermicidal foam/gel/film/cream/suppository, and all boys must also not donate sperm during the study and for 6 months after receiving the last dose of study drug.
15. Subjects' screening laboratory tests must meet the following criteria:
   a. Hemoglobin: ≥8.0 g/dL (SI: ≥80 g/L; girls and boys, ages 2 to 11)
      ≥8.5 g/dL (SI: ≥85 g/L; girls, ages 12 to 18)
      ≥9.0 g/dL (SI: ≥90 g/L; boys, ages 12 to 18)
   b. White blood cells (WBCs)≥3.0×10³ cells/μL (SI: ≥3.0×10⁹ cells/L)
   c. Neutrophils≥1.5×10³ cells/μL (SI: ≥1.5×10⁹ cells/L)
   d. Platelets≥140×10³ cells/μL (SI: ≥140×10⁹ cells/L)
   e. Serum transaminase levels not exceeding 1.2× the upper limit of normal for the central laboratory:
   Aspartate aminotransferase (AST)
      ≤67 IU/L (girls, ages 2 to <4)
      ≤58 IU/L (girls, ages 4 to <7)
      ≤48 IU/L (girls, ages 7 to 18)
      ≤83 IU/L (boys, ages 2 to <4)
      ≤71 IU/L (boys, ages 4 to <7)
      ≤48 IU/L (boys, ages 7 to 18)
   Alanine aminotransferase (ALT)
      ≤41 IU/L (girls, ages 2 to 18)
      ≤41 IU/L (boys, ages 2 to <10)
      ≤52 IU/L (boys, ages 10 to 18)
   f. Serum creatinine not to exceed:
   0.5 mg/dL (SI: 44 μmol/L; ages 2 to 5)
   0.7 mg/dL (SI: 62 μmol/L; ages 6 to 10)
   1.0 mg/dL (SI: 88 μmol/L; ages 11 to 12)
   1.2 mg/dL (SI: 106 μmol/L; ages≥13)
16. Subjects must be up to date with all immunizations in agreement with current local immunization guidelines for immunosuppressed subjects before Week 0.
17. A parent or guardian should accompany the subject to each study visit until the subject reaches the age of 18 years.
18. The subject and his/her parent (if applicable) must be able to adhere to the study visit schedule, and understand and comply with other protocol requirements.
19. Subject must be willing and able to adhere to the prohibitions and restrictions specified in this protocol.
20. Each subject (or their legally acceptable representative) must sign an ICF indicating that he or she understands the purpose of and procedures required for the study and are willing to participate in the study. Assent is also required of children capable of understanding the nature of the study (typically 7 years of age and older and per local regulations) as described in Section 16.2.3, Informed Consent.

### 4.2. Exclusion Criteria

Any potential subject who meets any of the following criteria will be excluded from participating in the study.

Concomitant or previous medical therapies received:
1. Subject has initiated DMARDs and/or immunosuppressive therapy within 4 weeks prior to first study agent administration.
2. Subject has been treated with intra-articular, intramuscular or intravenous corticosteroids (including intramuscular corticotropin) during the 4 weeks before first study agent administration.
3. Subject has been treated with any therapeutic agent targeted at reducing IL-12 or IL-23, including but not limited to ustekinumab and ABT-874 within 3 months before first study agent administration.
4. Subject has been treated with natalizumab, efalizumab, or therapeutic agents that deplete B or T cells (eg, rituximab, alemtuzumab, or visilizumab) during the 12 months before first study agent administration, or has evidence at screening of persistent depletion of the targeted lymphocyte after receiving any of these agents.
5. Subject has been treated with alefacept within 3 months before first study agent administration.
6. Subject has been treated with abatacept within 8 weeks before first study agent administration.
7. Subject has been treated with leflunomide within 4 weeks before first study agent administration (irrespective of undergoing a drug elimination procedure), or have received leflunomide from 4 to 12 weeks before first study agent administration and have not undergone a drug elimination procedure.
8. Subject has been treated with cytotoxic agents, including cyclophosphamide, nitrogen mustard, chlorambucil, or other alkylating agents.
9. Subject has received or is expected to receive any live viral or live bacterial vaccinations from 3 months before first study agent administration and up to 3 months after the last study agent administration.
10. Subject has had a BCG vaccination within 12 months of screening or is planned to receive BCG vaccination within 12 months following last study drug administration.
11. Subject has received IL-1ra (anakinra) within 1 week of the first study agent administration.
12. Subject has previously been treated with more than 2 therapeutic agents targeted at reducing TNFα, including, but not limited to, infliximab, etanercept, adalimumab, or certolizumab pegol.
13. If a subject has been previously treated with an anti-TNFα agent, the reason for discontinuation of the anti-TNFα agent cannot have been a severe or serious adverse event consistent with the class of anti-TNFα agents.
14. Subject has received adalimumab or certolizumab pegol within 6 weeks or has received etanercept within 4 weeks of the first dose of study agent.
15. Subject has received infliximab or tocilizumab within 8 weeks of the first administration of study agent.
16. Subject has ever received IV or SC golimumab.
17. Subject has received a Janus kinase (JAK) inhibitor, including but not limited to tofacitinib, within 2 weeks of the first dose of study agent.
18. Subject has received canakinumab within 4 months prior to first study dose administration.
19. Subject has current side effects related to MTX or conditions that would preclude treatment with MTX, including but not limited to liver cirrhosis, liver fibrosis, persistent elevations of ALT and AST (more than 3 of 5 tests elevated within 6-months period), MTX pneumonitis, severe mucosal ulcers, intractable nausea, vomiting/diarrhea, evidence of clinically significant bone marrow suppression, severe headaches, severe bone pain, or traumatic fractures.
20. Subject has received an investigational drug (including investigational vaccines) or used an invasive investigational medical device within 3 months or 5 half-lives, whichever is longer, before the planned first dose of study drug or is currently enrolled in an investigational study.

Infections or predisposition to infections:
21. Subject has a history of active granulomatous infection, including histoplasmosis or coccidioidomycosis, prior to screening. Refer to inclusion criterion (Section 4.1) for information regarding eligibility with a history of latent TB.
22. Subject tests positive for hepatitis B virus.
23. Subject is seropositive for antibodies to hepatitis C virus (HCV).
24. Subject has a known history of infection with human immunodeficiency virus (HIV).
25. Subject has had a nontuberculous mycobacterial infection or opportunistic infection (eg, cytomegalovirus, pneumocystis, or aspergillosis) within 6 months prior to screening.
26. Subject has a history of an infected joint prosthesis or has received antibiotics for a suspected infection of a joint prosthesis unless that prosthesis has been removed or replaced.
27. Subject has or has had a serious infection (including but not limited to hepatitis, pneumonia, or pyelonephritis), or have been hospitalized or received IV antibiotics for an infection during the 2 months before first study agent administration.
28. Subject has a history of or ongoing chronic or recurrent infectious disease, including, but not limited to, chronic renal infection, chronic chest infection (eg, bronchiectasis), sinusitis, recurrent urinary tract infection (eg, recurrent pyelonephritis), open, draining, or infected skin wound, or ulcer.
29. Subject has a chest radiograph within 3 months prior to the first administration of study agent that shows an abnormality suggestive of a malignancy or current active infection, including TB (if applicable).

Malignancy or increased potential for malignancy:
30. Subject has a known malignancy or a history of malignancy.
31. Subject has a history of lymphoproliferative disease, including lymphoma, or signs suggestive of possible lymphoproliferative disease, such as lymphadenopathy of unusual size or location, or clinically significant splenomegaly not consistent with NIA or systemic onset JIA without systemic symptoms.

Coexisting medical conditions or past medical history:
32. Subject has a history of severe progressive or uncontrolled liver or renal insufficiency; or significant cardiac, vascular, pulmonary, gastrointestinal, endocrine, neurologic, hematologic, psychiatric, or metabolic disturbances.
33. Subject has known allergies, hypersensitivity, or intolerance to golimumab or its excipients or subject has known allergies, hypersensitivity, or intolerance to immunoglobulins.
34. Subject has or has had a substance abuse (drug or alcohol) problem.
35. Subject has a history of macrophage activation syndrome.
36. Subject has another inflammatory disease that might confound the evaluation of benefit from golimumab therapy, including but not limited to systemic lupus erythematosus or Lyme disease.
37. Subject is incapacitated, largely or wholly bedridden, or confined to a wheelchair, or has little or no ability for age-appropriate self-care.
38. Subject has a known history of demyelinating diseases such as multiple sclerosis.
39. Subject has a history of, or concomitant diagnosis of, congestive heart failure.

### Other:

40. Subject has any condition for which, in the opinion of the investigator, participation would not be in the best interest of the subject (eg, compromise the well-being) or that could prevent, limit, or confound the protocol-specified assessments.
41. Subject is a girl who is pregnant, or breast-feeding, or planning to become pregnant while enrolled in this study or within 6 months after the last dose of study drug.
42. Subject is a boy who plans to father a child while enrolled in this study or within 6 months after the last dose of study drug.
43. Subject is unable or unwilling to undergo multiple venipunctures because of poor tolerability or lack of easy access.
44. Subject is an employee of the investigator or study site, with direct involvement in the proposed study or other studies under the direction of that investigator or study site, as well as family members of the employees or the investigator.
45. Subject has active uveitis within 3 months prior to screening.
46. Subject with BSA>3.0 m².

NOTE: Investigators should ensure that all study enrollment criteria have been met at screening. If a subject's status changes (including laboratory results or receipt of additional medical records) after screening but before the first dose of study drug is given such that he or she no longer meets all eligibility criteria, then the subject should be excluded from participation in the study. Section 17.4, Source Documentation, describes the required documentation to support meeting the enrollment criteria.

### 4.3. Prohibitions and Restrictions

Potential subjects must be willing and able to adhere to the following prohibitions and restrictions during the course of the study to be eligible for participation:
1. Subjects must not receive a live virus or live bacterial vaccination 3 months prior to screening, during the study, or within 3 months after the last administration of study agent.
2. Subjects must not receive a BCG vaccination for 12 months before screening, during the study or within 12 months after the last administration of study agent.
3. If sexually active and of childbearing potential, girls must remain on a highly effective method of birth control during the study and for 6 months after receiving the last administration of study agent, including the LTE phase of the study. Girls must not donate eggs (ova, oocytes) for the purposes of assisted reproduction during the study and for 6 months after receiving the last dose of study agent, including the LTE phase of the study.
4. If sexually active with a girl of childbearing potential and has not had a vasectomy, boys must use a double barrier method of birth control during the study and for 6 months after receiving the last administration of study agent, including the LTE phase of the study. Boys must not donate sperm and must agree not to plan a pregnancy or father a child during the study and for 6 months following the last administration of study agent, including the LTE phase of the study.
5. Intramuscular administration of corticosteroids for the treatment of pJIA is not allowed during the study. Corticosteroids administered by bronchial or nasal inhalation for treatment of conditions other than pJIA may be given as needed throughout the course of the study. For additional details, see Section 8.
6. Subjects must not receive investigational drugs, other immunosuppressants (such as, but not exclusively, cyclophosphamide), or other biologics for pJIA during the study.

### 5. Treatment Allocation and Blinding

This is an open-label study. All subjects will receive golimumab 80 mg/m² at Week 0, Week 4, and q8w (±3 days) through Week 28 and q8w (±1 week) up to Week 244.

As this is an open-label study, blinding procedures are not applicable.

### 6. Dosage and Administration

### 6.1. Golimumab

The study will have 1 active treatment group and all subjects will receive 80 mg/m² golimumab (maximum single dose 240 mg) IV infusions at Week 0, Week 4, and q8w (±3 days) through Week 28 and q8w (±1 week) thereafter through Week 244. The golimumab infusions will be prepared by a pharmacist under sterile conditions using golimumab 50 mg/4 mL liquid in vials and a 100 mL infusion bag of 0.9% saline. Subjects will receive 80 mg/m² golimumab IV infusions over 30±10 minutes. Infusions may be slowed down for evidence of infusion reactions as deemed appropriate by the investigator, and all changes in the infusion rate should be recorded in the CRF. Body surface area will be calculated at each visit and the dose of golimumab will be adjusted as needed to maintain the dose at 80 mg/m². Body surface area will be calculated using the Mosteller equation: BSA (m²)=([height (cm)×weight (kg)]/3600)^{1/2}. For additional details, see the Site IP Manual.

### 6.2. Methotrexate

Subjects will receive commercial MTX through Week 28 at the same BSA-based dose (10 to 30 mg/m² per week for subjects with BSA<1.67 m² or at least 15 mg/week for subjects with BSA≥1.67 m²) as at time of study entry. Absolute dose should remain stable from baseline through Week 28.

Every effort should be made to ensure that subjects remain on the same dose and route of administration of MTX through the Week 28 visit, unless intolerance or AEs due to MTX occur (Section 8). Guidelines for adjusting MTX dosage in the event of MTX toxicity are provided in the Trial Center File.

Subjects will also receive a total dose of commercial folic acid ≥5 mg weekly or folinic acid (at half the MTX dose) given the day after the weekly MTX dose. In children <12 years of age, the administration of folic acid or folinic acid will be at the discretion of the physician.

After Week 28, changes in MTX administration are permitted (eg, increase or decrease in dosage, change in route of administration, or discontinuation).

### 7. Treatment Compliance

The study site personnel will ensure compliance with the treatment assignments. Site personnel will administer the study infusion at each visit and record the amount of infusion given.

All subject CRFs will be monitored by a site monitor designated by the Sponsor. During these monitoring visits, all procedures will be evaluated for compliance with the protocol. Treatments that are administered outside of the scheduled windows, as well as missed visits, will be recorded on the CRF. Subject charts will be reviewed and compared with the data entries on the CRFs to ensure accuracy.

### 8. Prestudy and Concomitant Therapy

Prestudy JIA medications administered before the first dose of study agent must be recorded at screening. All concomitant therapies must be recorded throughout the study beginning with the administration of the first dose of the study drug.

All therapies (prescription or over-the-counter medications, including vaccines, vitamins, herbal supplements; non-pharmacologic therapies such as electrical stimulation and acupuncture) different from the study drug must be recorded in the CRF. Recorded information will include a description of the type of the drug, treatment period, dosing regimen, route of administration, and its indication. Modification of an effective pre-existing therapy should not be made for the explicit purpose of entering a subject into the study.

If using corticosteroids or NSAIDS, subjects must have been on stable doses of these medications prior to study entry per Inclusion Criterion 8 and 9 (Section 4.1). Subjects may have been previously treated with no more than 2 therapeutic agents targeted at reducing TNFα prior to study entry per Exclusion Criterion 12 (Section 4.2). Subjects may not have initiated or been treated with prohibited therapeutic agents as outlined in Exclusion Criteria 1 through 20 (Section 4.2).

Subjects must have received MTX for ≥2 months before screening. For subjects with BSA<1.67 m², the MTX dose must be between 10 to 30 mg/m² per week and stable for ≥4 weeks before screening. For subjects with BSA≥1.67 m², the MTX dose must be a minimum of 15 mg/week of MTX and must be stable for ≥4 weeks before screening. For exceptions to this rule, see Inclusion Criterion 7. Subjects (with the exception of those with sJIA) receiving corticosteroids at the time of study entry must have been receiving a stable dose for ≥2 weeks before screening, and that dose must have been ≤10 mg/day prednisone or prednisone equivalent or 0.20 mg/kg/day (whichever is lower). Subjects with systemic onset JIA but without systemic symptoms for ≥3 months must be on stable corticosteroids for 3 days before screening and not exhibit systemic symptoms. If receiving NSAID therapy, the dose must have been stable for ≥2 weeks before screening.

No changes should be made to background medications (ie, MTX, other DMARDs, corticosteroids, and NSAIDs) in terms of increases or decreases in dosage (eg, no more than 10 mg/day prednisone or no more than 0.20 mg/kg/day, whichever is lower) and/or route of administration between Weeks 0 and 28, unless there is a safety concern (eg, elevated liver function tests), which requires changes to background medications. After Week 28, subjects will be permitted to change/add MTX, other DMARDs, corticosteroids, and NSAIDs, including increases or decreases in dosage, changes of route of administration, or discontinuations from these classes of agents.

Intramuscular administration of corticosteroids for the treatment of pJIA is not allowed during the study. Corticosteroids administered by bronchial or nasal inhalation for treatment of conditions other than pJIA may be given as needed throughout the course of the study.

Every attempt should be made to avoid the use of IV corticosteroids. For subjects requiring short courses (2 weeks or less) of oral or IV corticosteroids for reasons such as prophylactic therapy prior to surgery (stress-dose corticosteroids) or therapy for limited infections, exacerbation of asthma, or for any condition other than pJIA, corticosteroid therapy should be limited to situations in which, in the opinion of the treating physician, there are no adequate alternatives and should be documented in the CRF.

Subjects may receive intra-articular injections of a corticosteroid, if clinically required, during the study up to Week 52. However, the number of intra-articular injections should be limited to 2 over any 24-week period. That is, if a subject has received 2 intra-articular injections and more than 24 weeks has elapsed, the subject may receive up to 2 additional intra-articular injections over another 24-week period.

After Week 52, the number of injected joints is no longer limited to 2 injections per 24 weeks. The Sponsor must be notified in advance (or as soon as possible thereafter) of any instances in which prohibited therapies are administered (Section 4.3).

### 9. Study Evaluations

### 9.1. Study Procedures

### 9.1.1. Overview

The Time and Events Schedules summarize the frequency and timing of efficacy, PK, immunogenicity, and safety measurements applicable to this study (Table 6, Table 7, and Table 8). All scheduled study visits should occur within ±3 days of the intended visit through Week 28 and ±1 week from Week 28 through Week 244. If the recommended acceptable window cannot be observed, the Sponsor must be contacted before scheduling a visit.

The Childhood Health Assessment Questionnaire (CHAQ) should be conducted before any tests, procedures, or other consultations for that visit to prevent influencing subjects' perceptions. For additional details, refer to the PRO user manual.

At every unscheduled visit, the investigator will perform the following evaluations:
Review of systems
Vital signs
TB questionnaire
Adverse events
Review of concomitant medications
Safety laboratory evaluations

Additional serum or urine pregnancy tests may be performed, as determined necessary by the investigator or required by local regulation, to establish the absence of pregnancy at any time during the subject's participation in the study.

The total blood volume to be collected from each subject for the study is approximately 149.4 mL (Table 1). Repeat or unscheduled samples may be taken for safety reasons or for technical issues with the samples.

TABLE 1
Approximate Volume of Blood to be Collected
From Each Subject Through Week 252

| | | No. of | Approximate |
|---|---|---|---|
| | Approximate | Samples | Total |
| | Volume per | per | Volume of |
| Type of Sample | Sample (mL) | Subject | Blood (mL)^{a,b} |
| Safety (including screening and | | | |
| posttreatment assessments) | | | |
| Hematology | 1.2 | 17 | 20.4 |
| Serum chemistry | 1.1 | 17 | 18.7 |
| Serology (hepatitis B and | 2.0 | 1 | 2.0 |
| hepatitis C) | | | |
| Serum β-hCG pregnancy tests | 1.1 | 1 | 1.1 |
| QuantiFERON ® (TB Gold test) | 3.0 | 6 | 18.0 |
| Rheumatoid factor | 1.1 | 1 | 1.1 |
| Anti-dsDNA antibody | 1.1 | 11 | 12.1 |
| ANA antibodies | 1.1 | 11 | 12.1 |
| Efficacy (CRP) | 1.1 | 24 | 26.4 |
| PK and immunogenicity | 2.5 | 15 | 37.5 |
| (antibodies to golimumab) | | | |
| Approximate Total | | | 149.4 |

^{a}Calculated as the number of samples multiplied by amount of blood per sample.
^{b}Repeat or unscheduled samples may be taken for safety reasons or technical issues with the samples.
Note:
An indwelling intravenous cannula may be used for blood sample collection.
Abbreviations: ANA = antinuclear antibodies; β-hCG = β-human chorionic gonadotropin; CRP = C-reactive protein; dsDNA = double-stranded deoxyribonucleic acid; PK = pharmacokinetic; TB = tuberculosis.

### 9.1.2. Screening Phase

After written informed consent/assent has been obtained, and within a period of 6 weeks before Week 0, all screening evaluations establishing subject eligibility will be performed. Subjects who meet all of the inclusion and none of the exclusion criteria will be enrolled in the study. Every effort should be made to adhere to the study Time and Events Schedule for each subject (Table).

Girls of childbearing potential must have a negative serum β-hCG pregnancy test at screening and a negative urine pregnancy test prior to each administration of study agent. Sexually active subjects must consent to use a highly effective method of contraception and continue to use contraception for the duration of the study and for 6 months after receiving the last dose of study agent. The method(s) of contraception used by each subject must be documented.

Subjects must undergo testing for TB at screening and their medical history assessment must include specific questions about a history of TB or known personal exposure to individuals with active TB. The subject should be asked about past testing for TB, including chest radiograph results and responses to tuberculin skin or other TB testing (Section 4.1).

Subjects with a negative QuantiFERON® (TB Gold test) result (and a negative tuberculin skin test result in countries in which the QuantiFERON® (TB Gold test) is not approved/registered or the tuberculin skin is mandated by local Health Authorities) are eligible to continue with screening procedures. Subjects with a newly identified positive QuantiFERON®-TB Gold (and/or tuberculin skin test) result must undergo an evaluation to rule out active TB and initiate appropriate treatment for latent TB. Appropriate treatment for latent TB is defined according to local country guidelines for immunocompromised patients. If no local country guidelines for immunocompromised patients exist, US guidelines must be followed, or the subject will be excluded from the study.

A subject whose first QuantiFERON® (TB Gold test) result is indeterminate must have the test repeated. In the event that the second QuantiFERON® (TB Gold test) result is also indeterminate, the subject may be enrolled without treatment for latent TB if active TB is ruled out, their chest radiograph shows no abnormality suggestive of TB (active or old, inactive TB), and the subject has no additional risk factors for TB as determined by the investigator. This determination must be promptly reported to the Sponsor's medical monitor and recorded in the subject's source documents and initialed by the investigator.

Retesting of an abnormal screening value that leads to exclusion is allowed only once using an unscheduled visit during the screening period to reassess eligibility. This should only be considered if there is no anticipated impact on subject safety.

9.1.3. Treatment Phase: Week 0 through Week 28

Beginning at Week 0, eligible subjects will receive 80 mg/m² golimumab administered as IV infusions over 30±10 minutes at Weeks 0, 4 and q8w (±3 days) through Week 28 (Section 6.1). Subjects will also receive commercial MTX weekly at least through Week 28 at the same BSA-based dosage as at time of study entry and commercial folic acid ≥5 mg weekly or folinic acid (at half the MTX dose) given the day after the MTX dose (Section 6.2). In children <12 years of age, the administration of folic acid or folinic acid will be at the discretion of the physician.

Subjects will have safety, efficacy, PK, and immunogenicity evaluations performed according to the Time and Events Schedule (Table). One additional sample for serum golimumab concentration for population PK will be collected from all subjects at any time between Weeks 0 and 8 other than at the time of the Week 0, Week 4, and Week 8 visits; this sample must be collected at least 24 hours prior to or after a study agent administration and must not be collected at a regularly scheduled visit (eg, Week 8).

9.1.4. Treatment Phase: After Week 28 through Week 52

After Week 28, subjects will continue to receive 80 mg/m² golimumab administered as IV infusions over 30±10 minutes q8w (±1 week) through Week 52 (Section 6.1). Subjects may also receive commercial MTX weekly at the same BSA-based dosage as at time of study entry and commercial folic acid >5 mg weekly or folinic acid if administered (at half the MTX dose; Section 6.2) given the day after the MTX dose; however, increases, decreases or discontinuations of MTX, other DMARDs, corticosteroids, and/or NSAIDs are permissible after Week 28. All changes and reasons for changes for these medications need to be documented in the eCRF.

Subjects will have safety, efficacy, PK, and immunogenicity evaluations performed according to the Time and Events Schedule (Table).

### End of Treatment/Early Withdrawal

If a subject discontinues study agent before Week 52, the subject should return approximately 8 weeks after the last administration of study agent for a final safety follow-up visit (Section 10.2). If a subject withdraws from study participation before Week 52, every effort should be made to obtain end-of-treatment assessments prior to the subject's withdrawal of consent.

9.1.5. Long-Term Extension Phase: After Week 52 through Week 252

Subjects who enter the long-term extension after the Week 52 visit will continue to receive 80 mg/m² golimumab administered as IV infusions over 30±10 minutes q8w (±1 week) through Week 244.

Subjects will have safety, efficacy, PK, and immunogenicity evaluations performed according to the Time and Events Schedules (Table 7 and Table). Subjects who discontinue study agent administration prior to Week 244 without withdrawing consent should return for a final safety follow-up visit approximately 8 weeks after their last study agent infusion (Section 10.2).

Subjects should continue to be evaluated for signs and symptoms of TB (Section 9.4).

### 9.2. Efficacy

### 9.2.1. Evaluations

The Time and Events Schedule summarizes the frequency and timing of efficacy measurements applicable to this study (Table, Table 7, and Table).

### 9.2.1.1. Joint Evaluation

Each of 75 joints will be evaluated for tenderness, and 68 joints will be evaluated for swelling and pain and limitation on motion according to the standard PRINTO/PRCSG joint evaluation. A consistent joint assessor, with at least 1 year of experience in performing joint assessment, will be designated at each study center to perform all joint assessments.

Training will be provided to a single consistent joint assessor from each site before the start of subject enrollment; the training is mandatory unless the site's joint assessor has taken certified training provided by PRINTO or PRCSG. If a consistent joint assessor was trained by the Sponsor in a previous clinical study, he or she may receive a waiver for this training. Documentation of Sponsor or PRINTO/PRCSG training will be maintained in the Trial Center File. If possible, the consistent joint assessor for the study should not be changed during the study. However, the assessor from each site who attends the consistent joint assessor training provided by the Sponsor may train 1 additional assessor at the site for coverage during their absences.

It is expected that any additional consistent joint assessors who are trained will also have 1 or more years of experience as joint assessors or be approved by the Sponsor. If the designated consistent joint assessor from the site trains any additional assessors at the site, a letter documenting the training should be filed in the site's Trial Center File. In addition, if more than 1 consistent joint assessor at a site performs joint assessments during the study, the names of all consistent joint assessors performing the joint evaluation at the site at each visit must be listed in the Trial Center File and documented in the source document.

It is preferable that the consistent joint assessor who performs the baseline joint assessments for a subject also performs the joint assessments for that subject for all subsequent visits through the final efficacy assessment at Week 244.

### Nonevaluable Joints

While it may be reasonable in clinical practice to identify as “nonevaluable” any joint which in the past or during study participation has been surgically altered (ie, prosthesis placement) or medically treated (ie, intra-articular injection), the designation of “nonevaluable” for the purposes of this study is slightly different. Joints should only be designated as “nonevaluable” by the consistent joint assessor in the ePRO device if it is physically impossible to assess the joint (ie, joint inaccessible due to a cast, joint not present due to an amputation, joint deformed so as to make it impossible to assess).

### 9.2.1.2. American College of Rheumatology Pediatric Response

The JIA ACR 30 response criteria⁵ is defined as a 30% improvement (ie, a decrease in score) from baseline in at least 3 of the following 6 components, with worsening of 30% or more in no more than 1 of the following components:
Physician Global Assessment of Disease Activity
Parent/Subject Assessment of Overall Well-being
Number of active joints (defined as either swelling, or in absence of swelling, limited range of motion associated with pain on motion or tenderness)
Number of joints with limited range of motion
Physical function by CHAQ
CRP

The JIA ACR 50 response, the JIA ACR 70 response, and the JIA ACR 90 response are defined as a 50% improvement, a 70% improvement, and a 90% improvement from baseline, respectively, in at least 3 of the above 6 components, with worsening of 30% or more in no more than 1 of the above components.

### Inactive Disease

Inactive disease is indicated by the presence of all of the following:
No joints with active arthritis
No fever, rash, serositis, splenomegaly, hepatomegaly, or generalized lymphadenopathy attributable to JIA
No active uveitis
Normal CRP (≤0.287 mg/dL for subjects without underlying inflammatory disease)
Physician Global Assessment of Disease Activity indicating no active disease (<5 mm)
Duration of morning stiffness <15 minutes

### Clinical Remission While on Medication for JIA

Clinical remission while on medication for JIA is defined as inactive disease at each visit for a period of ≥6 months while on medication.

### 9.2.1.3. Physician Global Assessment of Disease Activity

The Physician Global Assessment of Disease Activity is a 100 mm VAS. Physicians are to complete the VAS that has them assess the patient's current arthritis activity. The anchors of the scale are “no arthritis activity” to “extremely active arthritis.” Lower scores indicate less disease activity. The process for including this measure in the core set of variables for the assessment of children has been captured in the literature.⁵

### 9.2.1.4. Childhood Health Assessment Questionnaire

The functional status of subjects will be assessed by the CHAQ.²¹ Parents/subjects will complete this questionnaire to assess the degree of difficulty the subject has in accomplishing tasks in 8 functional areas (dressing and grooming, arising, eating, walking, hygiene, reaching, gripping, and activities of daily living). Responses in each functional area are scored as 0 (without any difficulty), 1 (with some difficulty), 2 (with much difficulty), 3 (unable to do), or 4 (not applicable). Lower scores are indicative of improved functioning and task performance in specific functional areas.

Additionally, the CHAQ includes 2 VAS questions—one used to assess the subject's level of pain, and one used to assess the subject's overall well-being. Properties of the CHAQ have been evaluated and its validity assessed.²¹ The CHAQ has been shown to be responsive to disease change.²¹ A decrease of 0.188 has been determined to be a meaningful clinical improvement.¹

### Parent/Subject Assessment of Pain

Pain will be assessed as average pain experienced by the subject during the past week using a VAS scale that ranges from “no pain” (0 mm) to “very severe pain” (100 mm). This assessment should be completed by the parents (caregiver)/subjects prior to the tender and swollen joint examination.

### Parent/Subject Assessment of Overall Well-Being

The Parent/Subject Assessment of Overall Well-being is a 0-100 mm VAS. Parents/subjects will complete the VAS that asks them to consider all the ways arthritis impacts their child/themselves and then indicate how the subject is doing. The anchors of the scale are “very well” (0 mm) to “very poor” (100 mm). Lower scores indicate better well-being. The process for including this measure in the core set of variables for the assessment of children has been captured in the literature.⁵

Subjects who are 15 to <18 years of age at study entry may complete the CHAQ jointly with the parent/caregiver. Preferably, the same individual (eg, parent, caregiver, or subject) who completes the assessment at the start of the study should complete the assessment throughout the study.

### 9.2.1.5. C-Reactive Protein

C-reactive protein has been demonstrated to be useful as a marker of inflammation in patients with pJIA and is part of the JIA ACR 30 core assessments. C-reactive protein will be assayed by a central laboratory using a validated, high-sensitivity CRP assay.

### 9.2.1.6. Juvenile Arthritis Disease Activity Score (JADAS)

Recently, a composite disease activity score for pJIA, the Juvenile Arthritis Disease Activity Score (JADAS), was developed; in validation analyses it was found to have good metrologic properties, including the ability to predict disease outcome. The JADAS (modified for using CRP) is computed by assessing the following variables: (1) physician global rating of overall disease activity, measured on a 100-mm horizontal VAS (0 no activity; 100 maximum activity for both VAS); (2) parent/child ratings of well-being and pain, assessed on a 21-Numbered Circle and 100-Millimeter Horizontal Line Visual Analog Scales⁴, (3) number of active joints, assessed in 71, 27, or 10 joints (JADAS 71, JADAS 27, and JADAS 10, respectively); and (4) CRP was truncated to a 0 scale according to the following formula: (CRP [mg/L]-10/10), similar to the truncated ESR used in JADAS-ESR. Before calculation, CRP values<10 mg/L are converted to 10 and CRP values>110 mg/L are converted to 110.¹³

The JADAS is calculated as the sum of the scores of its 4 components, which yields a global score of 0 to 101, 0 to 57, and 0 to 40 for the JADAS 71, and JADAS 27, and JADAS 10, respectively.

The state of JADAS 10, 27, and 71 minimal disease activity^{2,11} was defined as the presence of all of the following: Physician Global Assessment of Disease Activity of ≤3.5, parent's global rating of well-being of ≤2.5, and swollen joint count of ≤1 in patients with polyarthritis.

The criteria for JADAS inactive disease is defined as a total JADAS score of ≤1.

### 9.2.2. Endpoints

### Primary Endpoint

The primary endpoint in this study is PK exposure at Week 28 (the trough concentrations at Week 28) and the Bayesian AUC_{SS} over one dosing interval of 8 weeks (from population PK modeling and simulation).

### Major Secondary Endpoints

Major secondary endpoints include: PK exposure at Week 52 (the trough concentrations at Week 52) and the Bayesian AUC_{SS} at Week 52 (from population PK modeling and simulation)

### Other Endpoints

Other endpoints include:
The proportions of subjects who are JIA ACR 30, 50, 70, and 90 responders over time
The change from baseline in CHAQ over time
CRP concentrations over time
The proportion of subjects who have inactive disease over time
The proportion of subjects in clinical remission on medication for pJIA over time
The improvement from baseline in the pJIA core set at each visit
The proportions of subjects who are JIA ACR 30, 50, 70, and 90 responders by disease subtype, and/or age over time through Week 52
The change from baseline in JADAS 10, 27, and 71 scores over time
The proportion of subjects who achieve JADAS 10, 27, and 71 minimal disease activity over time

### 9.3. Pharmacokinetics and Immunogenicity

### 9.3.1. Evaluations

Serum samples will be used to evaluate the PK, as well as the immunogenicity of golimumab (antibodies to golimumab). Venous blood samples will be collected and each serum sample will be divided into 3 aliquots (1 each for pharmacokinetics, antibodies to study drug, and a back-up). Subject confidentiality will be maintained. The sample should be drawn from a different arm than the IV line, or if using an IV line that is also being used to deliver medication, the line should be flushed and cleared of any residual medication that may be remaining prior to each PK sample being drawn. When using an IV line to draw PK samples, the first 1 mL of blood should be drawn and discarded prior to obtaining the sample. Intravenous line maintenance should be followed as per the standard of care. At visits where serum concentration and antibodies to golimumab will be evaluated, 1 blood draw of sufficient volume can be used.

### 9.3.2. Analytical Procedures

### Pharmacokinetics

Serum samples will be analyzed to determine concentrations of golimumab using a validated, specific, and sensitive method by or under the supervision of the Sponsor.

### Immunogenicity

The detection and characterization of antibodies to golimumab will be performed using a validated assay method by or under the supervision of the Sponsor. All samples collected for detection of antibodies to golimumab will also be evaluated for golimumab serum concentration to enable interpretation of the antibody data.

### 9.3.3. Pharmacokinetic Parameters

Serum golimumab concentrations will be evaluated at Weeks 0, 4, 8, 12, 20, 28, 52, 100, 148, 196, and 244 and summarized over time.

Pre-infusion (immediately before infusion) and post-infusion (1 hour after infusion) samples will be drawn at Weeks 0, 4, and 12, and an additional random population PK sample will be drawn at any time between Weeks 0 and 8 other than at the time of the Week 0, Week 4, and Week 8 visits and collected at least 24 hours prior to or after study agent administration. For each of the remaining visits, only 1 sample for serum golimumab will be collected, which should be collected prior to the infusion if an infusion of the study agent is administered at that visit. Post-infusion samples should be drawn from a different arm than the IV infusion line, or the IV infusion line must be flushed and cleared of any residual medication that may be remaining and 1 mL of blood should be drawn and discarded prior to obtaining the sample if using the same access line as was used for drug administration.

A population PK analysis with data through Week 28 will be performed to characterize the PK of golimumab as well as to identify important covariates of PK in the pediatric population with pJIA. Additionally the population PK model will be used to assess the similarity of the PK in pediatrics and adults. The clearance and volume of distribution will be estimated using a NONMEM approach. In addition, an exposure-response analysis will be performed to explore and characterize the relationship between exposure and efficacy.

### 9.3.4. Immunogenicity Assessments (Antibodies to Golimumab)

Antibodies to golimumab will be evaluated in serum samples collected from all subjects according to the Time and Events Schedule (ie, Weeks 0, 4, 8, 12, 28, 52, 100, 148, 196, and 244). Additionally, serum samples should also be collected at the final visit from subjects who are discontinued from treatment or withdrawn from the study. These samples will be tested by the Sponsor or Sponsor's designee.

Serum samples will be screened for antibodies binding to golimumab and the titer of confirmed positive samples will be reported. Other analyses may be performed to verify the stability of antibodies to golimumab and/or further characterize the immunogenicity of golimumab.

The incidence of antibodies to golimumab during the study will be determined.

### 9.4. Safety Evaluations

Any clinically relevant changes occurring during the study must be recorded on the Adverse Event section of the CRF.

Any clinically significant abnormalities persisting at the end of the study/early withdrawal will be followed by the investigator until resolution or until a clinically stable endpoint is reached.

The study will include the following evaluations of safety and tolerability according to the time points provided in the Time and Events Schedules:

### Adverse Events

Adverse events will be reported by the subject (or, when appropriate, by a caregiver, surrogate, or the subject's legally acceptable representative) for the duration of the study. Adverse events will be followed by the investigator as specified in Section 12, Adverse Event Reporting.

### Clinical Laboratory Tests

Blood samples for serum chemistry and hematology will be collected. The investigator must review the laboratory report, document this review, and record any clinically relevant changes occurring during the study in the adverse event section of the CRF. The laboratory reports must be filed with the source documents.

The following tests will be performed by the central laboratory:
Hematology Panel

| | |
|---|---|
| hemoglobin | WBC (neutrophils, lymphocytes, monocytes, |
| | eosinophils, basophils [%, absolute]) |
| hematocrit | platelet count |
| RBC | mean corpuscular volume |
| mean corpuscular | mean corpuscular hemoglobin concentration |
| hemoglobin | |
| RBC morphology | WBC morphology (if present) |

Serum Chemistry Panel

| | |
|---|---|
| sodium | total bilirubin |
| potassium | bilirubin (direct and indirect) |
| urea nitrogen | calcium |
| creatinine | phosphorous |
| glucose | albumin |
| AST | total protein |
| ALT | |
| alkaline phosphatase | uric acid |
| bicarbonate | chloride |

Serum pregnancy testing for girls of childbearing potential will be conducted at screening.
Urine pregnancy testing for girls of childbearing potential will be performed according to the Time and Events Schedules.
Additional serum or urine pregnancy tests may be performed, as determined necessary by the investigator or required by local regulation, to establish the absence of pregnancy throughout the study.
Serology for hepatitis B surface antigen (HBsAg), hepatitis B surface antibody (anti-HBs), and hepatitis B core antibody (anti-HBc total) at screening.
Serology for HCV antibody at screening.

### Vital Signs

Pulse/heart rate, respiratory rate, temperature, and blood pressure measurements will be performed according to the Time and Events Schedules (Table, Table 7, and Table).

Vital signs should be taken pre-infusion; at 15 and 30 minutes (15-minute intervals during the infusion); and at 60 and 90 minutes (during the 1-hour observation period following the infusion).

### Physical Examination

Physical examinations, including a skin exam at every physical examination and Tanner staging at least every 6 months for sexual maturity will be performed according to the Time and Events Schedule. Review of systems will be performed at all visits to evaluate for new symptomatology and if necessary, full physical examination may be performed at investigator discretion. Any clinically significant abnormalities persisting at the end of the study will be followed by the investigator until resolution or until reaching a clinically stable endpoint.

### Height and Body Weight

Height will be measured at screening, and all timepoints specified in the Time and Events Schedule. Weight will be measured at the timepoints specified in the Time and Events Schedule, using a calibrated scale at each weight measurement. Subjects will be instructed to remove shoes and outdoor apparel and gear.

### Uveitis Evaluations

All subjects will be assessed for new-onset uveitis at screening and at least every 6 months thereafter by the investigator based on physical examination and interview. This consists of an assessment of signs and symptoms of uveitis, including, but not limited to, eye redness, light sensitivity, changes in vision, and floaters. Based upon changing clinical standards, examinations may be more frequent.

In addition, all subjects are required to have slit lamp evaluations performed by an ophthalmologist/optometrist during the study at intervals (based on JIA subtype, ANA test results, age at JIA onset, and JIA duration) as specified.

If a subject develops uveitis during the study, the subject's continued participation in the study is at the discretion of the investigator and Sponsor.

### Infusion Reaction Evaluations

Before an infusion is started, the appropriate personnel, medications (eg, epinephrine, inhaled beta agonists, antihistamines and corticosteroids), and other requirements to treat anaphylaxis should be available. The subject may be premedicated with prophylactic drugs (eg, diphenhydramine) prior to starting the infusion based on investigator's discretion but this is not mandatory. However, corticosteroids for prophylaxis are not allowed. Premedications should be recorded in the eCRF.

The investigator or qualified designee will evaluate the subject for infusion reactions according to the Time and Events Schedule.

An infusion reaction is any unfavorable or unintended sign that occurs during the infusion or within 1 hour of completion of the infusion. All subjects must be carefully observed for symptoms of an infusion reaction. Subjects will be observed for at least 60 minutes after completion of the IV administration of study agent for symptoms of an infusion reaction. If an infusion reaction is observed, the subject should be treated at the investigator's discretion.

The investigator will record the infusion reaction in the AE page. If no infusion reaction is observed, the investigator will note this in the subject's medical records (source data).

### Allergic Reactions

Throughout the study, all subjects must be observed carefully for symptoms of an allergic reaction (eg, urticaria, itching, hives) for at least 60 minutes after the completion of the infusion. If mild or moderate allergic reaction is observed, acetaminophen or NSAIDs and diphenhydramine at approved pediatric doses may be administered.

Subjects with severe reactions following an infusion that result in bronchospasm with wheezing and/or dyspnea and require ventilatory support, or symptomatic hypotension with a decrease in systolic blood pressure greater than 40 mm mercury (Hg), will not be permitted to receive any additional study agent infusions. In the case of such reactions, appropriate medical treatment should be administered.

### Early Detection of Active Tuberculosis

To aid in the early detection of TB, reactivation, or new TB infection during study participation, subjects must be evaluated for signs and symptoms of active TB at scheduled visits (refer to Time and Events Schedule) or by telephone contact approximately every 8 to 12 weeks. The following series of questions is suggested for use during the evaluation.
“Has your child had a new cough of >14 days' duration or a change in a chronic cough?”
“Has your child had any of the following symptoms”:
   Persistent fever?
   Unintentional weight loss?
   Night sweats?”
“Has your child had close contact with an individual with active TB?” (If there is uncertainty as to whether a contact should be considered “close,” a physician specializing in TB should be consulted.)

If the evaluation raises suspicion that a subject may have TB reactivation or new TB infection, study agent administration should be interrupted and an immediate and thorough investigation should be undertaken, including, where possible, consultation with a physician specializing in TB.

Investigators should be aware that TB reactivation in immunocompromised subjects may present as disseminated disease or with extrapulmonary features. Subjects with evidence of active TB must immediately discontinue study agent and should be referred for appropriate treatment.

Annual QuantiFERON®-TB Gold (and tuberculin skin) testing is not required for subjects with a history of latent TB, and ongoing treatment for latent TB, or documentation of having completed adequate treatment for TB.

Subjects who experience close contact with an individual with active TB during the conduct of the study must have a repeat chest radiograph, a repeat QuantiFERON® (TB Gold test), a repeat tuberculin skin test in countries in which the QuantiFERON® (TB Gold test) is not approved/registered, and, if possible, referral to a physician specializing in TB to determine the subject's risk of developing active TB and whether treatment for latent TB is warranted. The QuantiFERON® (TB Gold test) (and tuberculin skin test) does not need to be repeated for subjects with a history of latent TB, and ongoing treatment for latent TB, or documentation of having completed adequate treatment for TB. If the QuantiFERON® (TB Gold test) result is indeterminate, the test should be repeated as outlined in Section 9.1.2. Subjects should be encouraged to return for all subsequent scheduled study visits according to the protocol.

### 9.5. Sample Collection and Handling

The actual dates and times of sample collection must be recorded in the CRF or laboratory requisition form.

Refer to the Time and Events Schedule for the timing and frequency of all sample collections.

Instructions for the collection, handling, storage, and shipment of samples are found in the laboratory manual that will be provided. Collection, handling, storage, and shipment of samples must be under the specified, and where applicable, controlled temperature conditions as indicated in the laboratory manual.

### 10. Subject Completion/Withdrawal

### 10.1. Completion

A subject will be considered to have completed the main study if he or she has completed assessments at Week 52. A subject will be considered to have completed the long-term extension if he or she has completed assessments at Week 252.

### 10.2. Discontinuation of Study Treatment

If a subject's study treatment must be discontinued before the end of the treatment regimen, this will not result in automatic withdrawal of the subject from the study.

A subject's study treatment should be permanently discontinued if any of the following occur:
The investigator believes that for safety reasons (eg, adverse event) it is in the best interest of the subject to discontinue study treatment.
The subject becomes pregnant.
Reaction resulting in bronchospasm (both new-onset study agent-related and severe exacerbation of pre-existing asthma) with and without wheezing, and/or dyspnea requiring ventilatory support, and/or symptomatic hypotension that occurs following a study agent administration.
Reaction resulting in myalgia and/or arthralgia with fever and/or rash (suggestive of serum sickness and not representative of signs and symptoms of other recognized clinical syndromes) occurring 1 to 14 days after an infusion of study agent. These may be accompanied by other events including pruritus, facial, hand, or lip edema, dysphagia, urticaria, sore throat, and/or headache.
Opportunistic infection.
Malignancy.
The subject develops congestive heart failure at any time during the trial.
Demyelinating disease.
The subject withdraws consent for administration of study agent.
The initiation of protocol-prohibited medications.
Subject is deemed ineligible according to the following TB screening criteria.
   A diagnosis of active TB is made.
   A subject has symptoms suggestive of active TB based on follow-up assessment questions and/or physical examination, or has had recent close contact with a person with active TB, and cannot or will not continue to undergo additional evaluation.
   A subject undergoing evaluation has a chest radiograph with evidence of current active TB and/or a positive QuantiFERON® (TB Gold test) result (or a positive tuberculin skin test result in countries in which the QuantiFERON® (TB Gold test) is not approved/registered or the tuberculin skin test is mandated by local Health Authorities), unless active TB can be ruled out and appropriate treatment for latent TB can be initiated prior to the next administration of study agent and continued to completion. Indeterminate QuantiFERON® (TB Gold test) results should be handled as in Section 9.1.2. Subjects with persistently indeterminate QuantiFERON® (TB Gold test) results may continue without treatment for latent TB if active TB is ruled out, their chest radiograph shows no abnormality suggestive of TB (active or old, inactive TB) and the subject has no additional risk factors for TB as determined by the investigator. This determination must be promptly reported to the Sponsor's medical monitor and recorded in the subject's source documents and initialed by the investigator.
   A subject receiving treatment for latent TB discontinues this treatment prematurely or is noncompliant with the therapy.

All subjects who discontinue study agent infusions during the study will be followed for approximately 8 weeks after the last infusion is administered.

Note: The visit that is approximately 8 weeks after the last study agent infusions is referred to as the “final safety follow-up visit,” which may occur at a scheduled or an unscheduled visit.

Subjects who discontinue study agent infusions but do not terminate study participation will have the following assessments performed at the final safety follow-up visit:
Safety evaluations (vital signs, review of systems, AE review, TB evaluation, uveitis evaluation, and the collection of a blood sample for routine laboratory analyses and determination of the presence of ANA/anti-double-stranded deoxyribonucleic acid (dsDNA) antibodies and antibodies to golimumab).
Concomitant medication review.
Efficacy evaluations (joint assessments, JIA assessments, and collection of blood sample for CRP).
Blood samples drawn for measurement of golimumab concentration for all subjects at the final safety follow-up visit.

If a subject discontinues study treatment before the end of the study, assessments should be obtained approximately 8 weeks after the last infusion of study agent.

10.3. Withdrawal from the Study

A subject will be withdrawn from the study for any of the following reasons:
Lost to follow-up
Withdrawal of consent
Death

If a subject discontinues study treatment before the end of the study, end-of-treatment assessments should be obtained approximately 8 weeks after the last infusion of study agent at the final safety follow-up visit.

If a subject is lost to follow-up, every reasonable effort must be made by the study site personnel to contact the subject and determine the reason for discontinuation/withdrawal. The measures taken to follow-up must be documented.

When a subject withdraws before completing the study, the reason for withdrawal is to be documented in the CRF and in the source document. Study drug assigned to the withdrawn subject may not be assigned to another subject. Subjects who withdraw will not be replaced.

If a subject withdraws from the study before the end of the study, end-of-treatment assessments should be obtained prior to the withdrawal of consent.

### 11. Statistical Methods

Statistical analysis will be done by the Sponsor or under the authority of the Sponsor. A general description of the statistical methods to be used to analyze the efficacy and safety data is outlined below. Specific details will be provided in the Statistical Analysis Plan.

In general, descriptive statistics, such as mean, median, standard deviation, interquartile range, minimum and maximum for continuous variables, and counts and percentages for categorical variables will be used to summarize data.

### 11.1. Subject Information

All subjects who are enrolled in the study will have baseline descriptive statistics provided.

Subject baseline data, demographic and baseline disease characteristics will be summarized. The baseline measurement is defined as the closest measurement taken before the time of the Week 0 study agent administration.

Demographics and subject baseline disease characteristics and prior medication data will be summarized for all subjects who have been enrolled in the study, whether or not they have received study agent administration. Pharmacokinetic data will be summarized for all subjects who had received at least 1 administration of study agent. Efficacy analyses will be summarized for all subjects enrolled in the study unless otherwise specified. Safety assessments will be summarized for all treated subjects.

### 11.2. Sample Size Determination

The sample size determination is not based on statistical considerations. For the purpose of determining sample size of this study, the variability of PK in pediatric populations was considered. The goal is to have a sample size that will be sufficient to build a population PK and, if feasible, an exposure-response model. Additionally, a sample size that will provide reasonable safety assessments was also taken into consideration. With these considerations, a sample size of approximately 120 subjects has been chosen assuming that if 20 subjects were to drop out or if they do not provide PK samples, a sample size of approximately 100 subjects is thought to be sufficient to build a population PK model, given the sparse sampling of PK time points, as well as provide 1 year of safety data from approximately 100 subjects.

### 11.3. Efficacy Analyses

### Primary Endpoint Analysis

No primary efficacy endpoint analysis is planned.

### Major Secondary Endpoints Analyses

No major secondary efficacy endpoints analyses are planned.

### Other Efficacy Endpoints

The following will be summarized for all subjects enrolled in the study:
The proportion of subjects who are JIA ACR 30, 50, 70, and 90 responders over time
The proportion of subjects who have inactive disease over time
The proportion of subjects in clinical remission on medication for pJIA (ACR criteria) over time
The improvement from baseline in the pJIA core set over time
The proportions of subjects who are JIA ACR 30, 50, 70, and 90 responders by disease subtype, and/or age over time through Week 52
The change from baseline in CHAQ over time
CRP concentrations over time
The change from baseline in JADAS 10, 27, and 71 scores over time
The proportion of subjects who achieve JADAS 10, 27, and 71 minimal disease activity over time

### 11.4. Pharmacokinetic Analyses

The primary objective of this study is to characterize golimumab PK exposure (the trough concentrations at Weeks 28 and the Bayesian AUC_{SS} over a dosing interval of 8 weeks from population PK modeling and simulation) in the pJIA population.

Serum golimumab concentrations will be summarized over time. In addition, a population PK analysis on data through Week 28 will be performed to characterize the PK of golimumab as well as to identify and quantify important covariates of PK in the pediatric population with pJIA. Clearance and volume of distribution will be estimated using a NONMEM approach. Details will be provided in a population PK analysis plan and the results of the analysis will be presented in a separate report.

Measures of PK exposure will be graphically evaluated in the pediatric populations after administration of IV golimumab (including but not limited to steady-state Cₘₐₓ, Cₘᵢₙ and AUC) and compared to PK exposure from adults in CNTO148ART3001. Similarity between pediatric and adult subjects will be assessed by the generation of box plots from the population PK modeling via visual inspection in addition to the descriptive statistics of the observed concentrations.

Summary golimumab concentrations will be summarized and PK exposure will be evaluated through Week 52 and through the LTE.

### 11.5. Immunogenicity Analyses

The occurrence and titers of antibodies to golimumab during the study will be summarized over time for all subjects who receive an administration of golimumab and have appropriate samples collected for detection of antibodies to golimumab (ie, subjects with at least 1 sample obtained after their first golimumab administration).

### 11.6. Pharmacokinetic/Pharmacodynamic Analyses

The relationships between serum golimumab concentration and efficacy will be explored. A suitable PK/PD model will be explored and developed to describe the exposure-response relationship.

### 11.7. Safety Analyses

### Adverse Events

The verbatim terms used in the CRF by investigators to identify adverse events will be coded using the Medical Dictionary for Regulatory Activities (MedDRA). All reported adverse events with onset during the treatment phase (ie, treatment-emergent adverse events, and adverse events that have worsened since baseline) will be included in the analysis. For each adverse event, the percentage of subjects who experience at least 1 occurrence of the given event will be summarized by treatment group.

Summaries, listings, datasets, or subject narratives may be provided, as appropriate, for those subjects who die, who discontinue treatment due to an adverse event, or who experience a severe or a serious adverse event.

The following analyses will be used to assess the safety of subjects in this trial:
The occurrence and type of AEs
The occurrence and type of SAEs
The occurrence and type of reasonably related AEs
The occurrence of infusion reactions
The occurrence of ANA and anti-dsDNA antibodies
The occurrence of antibodies to golimumab
The occurrence of markedly abnormal laboratory (hematology and chemistry) parameters

### Clinical Laboratory Tests

Laboratory data will be summarized by type of laboratory test. Reference ranges and markedly abnormal results (specified in the Statistical Analysis Plan) will be used in the summary of laboratory data. Changes from baseline results will be presented in pre-versus posttreatment cross-tabulations (with classes for below, within, and above normal ranges). Frequency tabulations of the abnormalities will be made. A listing of subjects with any markedly abnormal laboratory results will also be provided.

### Vital Signs

Descriptive statistics of pulse/heart rate, respiratory rate, temperature, and blood pressure (systolic and diastolic) values and changes from baseline will be summarized at each scheduled time point in the Schedule of Events.

### 11.8. Interim Analysis

No interim analysis is planned.

### 11.9. Data Monitoring Committee

This is an open-label study, with all subjects receiving the same dosage of IV golimumab. Therefore, an external Data Monitoring Committee will not be utilized. Safety data will be routinely evaluated by the study's medical monitor and an internal Data Review Committee as defined in the DRC charter. In addition, the data may be reviewed by the Steering Committee.

### 12. Adverse Event Reporting

Timely, accurate, and complete reporting and analysis of safety information from clinical studies are crucial for the protection of subjects, investigators, and the Sponsor, and are mandated by regulatory agencies worldwide. The Sponsor has established Standard Operating Procedures in conformity with regulatory requirements worldwide to ensure appropriate reporting of safety information; all clinical studies conducted by the Sponsor or its affiliates will be conducted in accordance with those procedures.

### 12.1. Definitions

### 12.1.1. Adverse Event Definitions and Classifications

### Adverse Event

An adverse event is any untoward medical occurrence in a clinical study subject administered a medicinal (investigational or non-investigational) product. An adverse event does not necessarily have a causal relationship with the treatment. An adverse event can therefore be any unfavorable and unintended sign (including an abnormal finding), symptom, or disease temporally associated with the use of a medicinal (investigational or non-investigational) product, whether or not related to that medicinal (investigational or non-investigational) product. (Definition per International Conference on Harmonisation [ICH])

This includes any occurrence that is new in onset or aggravated in severity or frequency from the baseline condition, or abnormal results of diagnostic procedures, including laboratory test abnormalities.

Note: The Sponsor collects adverse events starting with the signing of the ICF (refer to Section 12.3.1, All Adverse Events, for time of last adverse event recording).

### Serious Adverse Event

A serious adverse event based on ICH and European Union Guidelines on Pharmacovigilance for Medicinal Products for Human Use is any untoward medical occurrence that at any dose:
Results in death
Is life threatening (The subject was at risk of death at the time of the event. It does not refer to an event that hypothetically might have caused death if it were more severe)
Requires inpatient hospitalization or prolongation of existing hospitalization
Results in persistent or significant disability/incapacity
Is a congenital anomaly/birth defect
Is a suspected transmission of any infectious agent via a medicinal product
Is Medically Important*

*Medical and scientific judgment should be exercised in deciding whether expedited reporting is also appropriate in other situations, such as important medical events that may not be immediately life threatening or result in death or hospitalization but may jeopardize the subject or may require intervention to prevent one of the other outcomes listed in the definition above. These should usually be considered serious.

If a serious and unexpected adverse event occurs for which there is evidence suggesting a causal relationship between the study drug and the event (eg, death from anaphylaxis), the event must be reported as a serious and unexpected suspected adverse reaction even if it is a component of the study endpoint (eg, all-cause mortality).

### Unlisted (Unexpected) Adverse Event/Reference Safety Information

An adverse event is considered unlisted if the nature or severity is not consistent with the applicable product reference safety information.

For MTX, which has a marketing authorization, the expectedness of an adverse event will be determined by whether or not it is listed in the package label supplied by the drug's manufacturer in that country.

Adverse Event Associated with the Use of the Drug

An adverse event is considered associated with the use of the drug if the attribution is possible, probable, or very likely by the definitions listed in Section 12.1.2.

### 12.1.2. Attribution Definitions

### Not Related

An adverse event that is not related to the use of the drug.

### Doubtful

An adverse event for which an alternative explanation is more likely, eg, concomitant drug(s), concomitant disease(s), or the relationship in time suggests that a causal relationship is unlikely.

### Possible

An adverse event that might be due to the use of the drug. An alternative explanation, eg, concomitant drug(s), concomitant disease(s), is inconclusive. The relationship in time is reasonable; therefore, the causal relationship cannot be excluded.

### Probable

An adverse event that might be due to the use of the drug. The relationship in time is suggestive (eg, confirmed by dechallenge). An alternative explanation is less likely, eg, concomitant drug(s), concomitant disease(s).

### Very Likely

An adverse event that is listed as a possible adverse reaction and cannot be reasonably explained by an alternative explanation, eg, concomitant drug(s), concomitant disease(s). The relationship in time is very suggestive (eg, it is confirmed by dechallenge and rechallenge).

### 12.1.3. Severity Criteria

An assessment of severity grade will be made using the following general categorical descriptors:

Mild: Awareness of symptoms that are easily tolerated, causing minimal discomfort and not interfering with everyday activities.

Moderate: Sufficient discomfort is present to cause interference with normal activity.

Severe: Extreme distress, causing significant impairment of functioning or incapacitation. Prevents normal everyday activities.

The investigator should use clinical judgment in assessing the severity of events not directly experienced by the subject (eg, laboratory abnormalities).

### 12.2. Special Reporting Situations

Safety events of interest on a Sponsor study drug that may require expedited reporting and/or safety evaluation include, but are not limited to:
Overdose of a Sponsor study drug
Suspected abuse/misuse of a Sponsor study drug
Inadvertent or accidental exposure to a Sponsor study drug
Any failure of expected pharmacologic action (ie, lack of effect) of a Sponsor study drug
Unexpected therapeutic or clinical benefit from use of a Sponsor study drug
Medication error involving a Sponsor product (with or without subject/patient exposure to the Sponsor study drug, eg, name confusion)

Special reporting situations should be recorded in the CRF. Any special reporting situation that meets the criteria of a serious adverse event should be recorded on the serious adverse event page of the CRF.

### 12.3. Procedures

### 12.3.1. All Adverse Events

All adverse events and special reporting situations, whether serious or non-serious, will be reported from the time a signed and dated ICF is obtained until completion of the subject's last study-related procedure (which may include contact for follow-up of safety). Serious adverse events, including those spontaneously reported to the investigator within 30 days of the end of the study, must be reported using the Serious Adverse Event Form. The Sponsor will evaluate any safety information that is spontaneously reported by an investigator beyond the time frame specified in the protocol.

All events that meet the definition of a serious adverse event will be reported as serious adverse events, regardless of whether they are protocol-specific assessments.

All adverse events, regardless of seriousness, severity, or presumed relationship to study drug, must be recorded using medical terminology in the source document and the CRF. Whenever possible, diagnoses should be given when signs and symptoms are due to a common etiology (eg, cough, runny nose, sneezing, sore throat, and head congestion should be reported as “upper respiratory infection”). Investigators must record in the CRF their opinion concerning the relationship of the adverse event to study therapy. All measures required for adverse event management must be recorded in the source document and reported according to Sponsor instructions.

The Sponsor assumes responsibility for appropriate reporting of adverse events to the regulatory authorities. The Sponsor will also report to the investigator (and the head of the investigational institute where required) all serious adverse events that are unlisted (unexpected) and associated with the use of the study drug. The investigator (or Sponsor where required) must report these events to the appropriate Independent Ethics Committee/Institutional Review Board (IEC/IRB) that approved the protocol unless otherwise required and documented by the IEC/IRB.

For all studies with an outpatient phase, including open-label studies, the subject must be provided with a “wallet (study) card” and instructed to carry this card with them for the duration of the study indicating the following:
Study number
Statement, in the local language(s), that the subject is participating in a clinical study
Investigator's name and 24-hour contact telephone number
Local Sponsor's name and 24-hour contact telephone number (for medical staff only)
Site number
Subject number
Any other information that is required to do an emergency breaking of the blind

### 12.3.2. Serious Adverse Events

All serious adverse events occurring during the study must be reported to the appropriate Sponsor contact person by study site personnel within 24 hours of their knowledge of the event.

Information regarding serious adverse events will be transmitted to the Sponsor using the Serious Adverse Event Form, which must be completed and signed by a physician from the study site, and transmitted to the Sponsor within 24 hours. The initial and follow-up reports of a serious adverse event should be made by facsimile (fax).

All serious adverse events that have not resolved by the end of the study, or that have not resolved upon discontinuation of the subject's participation in the study, must be followed until any of the following occurs:
The event resolves
The event stabilizes
The event returns to baseline, if a baseline value/status is available
The event can be attributed to agents other than the study drug or to factors unrelated to study conduct
It becomes unlikely that any additional information can be obtained (subject or health care practitioner refusal to provide additional information, lost to follow-up after demonstration of due diligence with follow-up efforts)

Suspected transmission of an infectious agent by a medicinal product will be reported as a serious adverse event. Any event requiring hospitalization (or prolongation of hospitalization) that occurs during the course of a subject's participation in a study must be reported as a serious adverse event, except hospitalizations for the following:
Hospitalizations not intended to treat an acute illness or adverse event (eg, social reasons such as pending placement in long-term care facility)
Surgery or procedure planned before entry into the study (must be documented in the CRF). Note: Hospitalizations that were planned before the signing of the ICF, and where the underlying condition for which the hospitalization was planned has not worsened, will not be considered serious adverse events. Any adverse event that results in a prolongation of the originally planned hospitalization is to be reported as a new serious adverse event.

The cause of death of a subject in a study within 2 months of the last dose of study drug, whether or not the event is expected or associated with the study drug, is considered a serious adverse event.

### 12.3.3. Pregnancy

All initial reports of pregnancy must be reported to the Sponsor by the study site personnel within 24 hours of their knowledge of the event using the appropriate pregnancy notification form. Abnormal pregnancy outcomes (eg, spontaneous abortion, stillbirth, and congenital anomaly) are considered serious adverse events and must be reported using the Serious Adverse Event Form. Any subject who becomes pregnant during the study must discontinue further study treatment.

Because the effect of the study drug on sperm is unknown, pregnancies in partners of male subjects included in the study will be reported by the study site personnel within 24 hours of their knowledge of the event using the appropriate pregnancy notification form.

Follow-up information regarding the outcome of the pregnancy and any postnatal sequelae in the infant will be required.
12.4. Events of Special Interest

Any newly identified malignancy or case of active TB occurring after the first administration of study agent(s) in subjects participating in this clinical study must be reported by the investigator according to the procedures in Section 12.3. Investigators are also advised that active TB is considered a reportable disease in most countries. These events are to be considered serious only if they meet the definition of a serious adverse event.

### 13. Study Drug Information

### 13.1. Physical Description of Study Drug

The test product, golimumab, will be supplied as a sterile liquid for IV infusion at a volume of 4 mL (50 mg, 12.5 mg/mL) in single-use vials. Each vial will contain golimumab in an aqueous medium of histidine, sorbitol and polysorbate 80 at pH 5.5. No preservatives are present. It will be manufactured and provided under the responsibility of the Sponsor.

MTX (oral or injectable) will not be supplied by the Sponsor but rather must be acquired from a commercial pharmacy.

### 13.2. Preparation, Handling, and Storage

Liquid study agent in glass vials will be supplied ready to use. At the study site, vials of golimumab solution must be stored in a secured refrigerator at controlled temperatures ranging from 2° C. to 8° C. (35.6° F. to 46.4° F.).

Pharmacokinetics, Efficacy, and Safety of Intravenous Golimumab in Patients with Juvenile Idiopathic Arthritis: Results from an Open-Label Phase 3 Study

### Patients and Methods

### Objectives

To assess pharmacokinetics (PK), efficacy, and safety of intravenous (IV) golimumab in pediatric patients with active polyarticular course juvenile idiopathic arthritis (poly-JIA) despite current methotrexate (MTX) therapy through 28 weeks of treatment and 52 weeks of treatment.

### Patients and Study Design

This was a Phase 3, open-label, single-arm, international study conducted in 33 centers in 9 countries. Eligible patients were 2 to <18 years of age weighing >15 kg at the time of screening and enrollment, with at least a 3-month history of poly-JIA and active arthritis (≥5 active joints) despite treatment with methotrexate (MTX; ≥10 mg/m²) for ≥2 months before screening, and onset of disease before their 16th birthday. Poly-JIA could include one of the following categories classified per JIA International League of Associations for Rheumatology (ILAR) classification criteria: extended oligoarticular JIA, rheumatoid factor (RF)-positive or RF-negative poly-JIA, systemic JIA with no systemic symptoms for ≥3 months, enthesitis-related arthritis, or polyarticular juvenile psoriatic arthritis.

All eligible patients received 80 mg/m² golimumab (maximum single dose of 240 mg) IV (over 30±10 minutes) at Weeks 0 and 4 and then every 8 weeks (q8w; ±3 days) through Week 28 and q8w (±1 week) through Week 52 (FIG. 18). Body surface area (BSA) was calculated at each visit, and the IV golimumab dose was adjusted as needed to maintain a dose of 80 mg/m². Commercial MTX was administered weekly at least through Week 28 at the same BSA-based dosage as at the time of study entry (10 to 30 mg/m² in patients with BSA<1.67 m² or a minimum dose of 15 mg per week in patients with BSA≥1.67 m²). After Week 28, patients were permitted to change or add MTX, other DMARDs, glucocorticoids, and NSAIDs. Patients who completed the study at Week 52 had the option to enter the long-term extension phase of the study that is now ongoing.

Patients had to be medically stable, could not have had active uveitis within 3 months prior to screening, and could not have a major concurrent medical condition. Patients were screened for tuberculosis, and those with evidence of active tuberculosis were excluded. Patients with latent tuberculosis were eligible if they were currently receiving treatment for latent tuberculosis. Patients of childbearing potential had to have a negative pregnancy test at screening and at each study visit, and sexually active patients had to agree to use a highly effective method of birth control.

If the patient was using glucocorticoids (≤10 mg/day or 0.20 mg/kg/day [whichever was less] for prednisone equivalent) or NSAIDs, the dose must have been stable for ≥2 weeks before the first administration of IV golimumab or screening, respectively. Up to 30% of patients could have prior exposure to ≤2 anti-TNF agents. Patients treated with a b-DMARD or a small molecule therapeutic prior to first administration of IV golimumab needed to observe specific washout periods. The washout period with respect to the first study agent administration was 1 week for interleukin (IL)-1ra; 2 weeks for Janus kinase inhibitors; 4 weeks for intra-articular, intramuscular, or IV glucocorticoids, leflunomide, etanercept, or initiation of DMARDs; 6 weeks for adalimumab or certolizumab pegol; 8 weeks for abatacept, infliximab, or tocilizumab; 3 months for IL-12/23 inhibitors, alefacept, live viral or bacterial vaccinations, investigational drugs, or medical devices; 4 months for canakinumab; and 12 months for Bacille Calmette-Guérin vaccination or agents that deplete B or T cells. Cytotoxic agents were prohibited.

An independent ethics committee or an institutional review board approved the study protocol for each site, and the study was conducted in accordance with the ethical principles that have their origin in the Declaration of Helsinki and that are consistent with Good Clinical Practice and applicable regulatory requirements. This study was registered at ClinicalTrials.gov (NCT02277444). Patients who were 7 years of age or older gave assent, and parents, a legal guardian, or a legally acceptable representative gave written informed consent for study participation.

### Study Assessments

Serum golimumab concentrations were measured at Weeks 0, 4, 8, 12, 20, 28, and 52. Pre-infusion and post-infusion samples were drawn at Weeks 0, 4, and 12, and an additional random population PK sample was drawn any time between Weeks 0 and 8 other than at the Week 0, 4, and 8 visits and collected at least 24 hours prior to or after study agent administration. Pre-infusion samples only were drawn at Weeks 8, 20, 28, and 52. Serum samples were analyzed using a validated, specific, and sensitive method.

Efficacy assessments included the JIA core set of measures (physician global assessment of overall disease activity [medical doctor (MD) global of disease activity; 0- to 10-cm visual analogue scale (VAS) from “no arthritis activity” to “extremely active arthritis” ], number of joints with active arthritis [swelling or, if no swelling is present, joints with limited range of motion and pain simultaneously], number of joints with limited range of motion, the cross-culturally adapted and validated version of the Childhood Health Assessment Questionnaire [CHAQ; including parent assessment of overall well-being and pain using VAS (0 to 10 cm)], and C-reactive protein [CRP; normal≤0.287 mg/dL for patients without underlying inflammatory disease]), and morning stiffness duration.

Safety assessments were performed at every visit and included routine laboratory evaluations. Any adverse events (AEs) were reported as a verbatim term and coded as per the Medical Dictionary for Regulatory Activities (MedDRA) Version 21.1. Antibodies to golimumab were evaluated in serum samples collected at Weeks 0, 4, 8, 12, 28, and 52 using a validated, highly sensitive drug-tolerant enzyme immunoassay method. Samples with antibodies specific to golimumab were classified as anti-drug antibody (ADA) positive. Patients with samples classified as ADA positive (treatment boosted [increased titer if baseline sample was ADA positive] or treatment induced) at any time after their first golimumab administration through Week 52 were classified as positive for antibodies to golimumab. Patients with baseline samples classified as ADA positive and without increased titer after treatment were classified as negative for antibodies to golimumab. The presence of anti-nuclear antibodies (ANA)/anti-double stranded DNA (dsDNA) antibodies was evaluated in serum samples collected at baseline, Week 24, and Week 52.

### Study Endpoints

The primary endpoints of this study were PK exposure at Week 28 (trough concentrations at Week 28) and model-predicted steady-state area under the curve (AUCₛₛ) over 1 dosing interval of 8 weeks (from population PK modeling and simulation) at Week 28. The major secondary endpoints were PK exposure at Week 52 (trough concentrations at Week 52) and model-predicted AUCₛₛ at Week 52.

Efficacy endpoints included the JIA American College of Rheumatology (ACR) 30, 50, 70, and 90 responses (defined as 30%, 50%, 70%, or 90% improvement from baseline in ≥3 without worsening of ≥30% in >1 of the remaining JIA core measures) calculated against the closest evaluation performed prior to the first IV golimumab administration (Week 0); a modified version of JIA ACR inactive disease (defined as no joints with active arthritis and no active uveitis; no fever, rash, serositis, splenomegaly, hepatomegaly, or generalized lymphadenopathy attributable to JIA; normal CRP; MD global ≤5 mm [no active disease]; and duration of morning stiffness of <15 minutes); clinical remission on medication for poly-JIA (defined as inactive disease at each visit for ≥6 months while on medication); and Juvenile Arthritis Disease Activity Score counting 71 joints (JADAS 71; cutoff values were >10.5 for high disease activity [HDA], 3.9 to 10.5 for moderate disease activity [MDA], 1.1 to 3.8 for low disease activity [LDA], and ≤1 for inactive disease [ID]).

### Statistical Analyses

This study followed the recommendation of the Consolidated Standards of Reporting Trials (CONSORT) statement, with results reported for the full analysis set. All patients who received at least 1 dose of study agent were included in the PK (if they had sufficient PK samples for analysis), efficacy, and safety analyses. A population PK analysis with data through Week 28 was performed to characterize the PK of golimumab and identify important covariates of PK in pediatric patients with poly-JIA. Population PK modeling was used to assess the similarity of the PK in pediatrics with adults. The clearance and volume of distribution were estimated using a nonlinear mixed-effects modeling (NONMEM) approach. Exposure-response analysis was also performed to explore and characterize the relationship between exposure and efficacy. Measures of PK exposure in the pediatric population were compared with PK exposure from a previous study in adults with RA who received IV golimumab 2 mg/kg at Weeks 0, 4, and q8w thereafter and were used as a reference population.

For the analysis of binary composite efficacy endpoints, imputation rules (non-responder imputation [NRI] for completely missing data and last observation carried forward [LOCF] for missing components) were used for imputing missing data as per the intention-to-treat (ITT) principle. There was no imputation for continuous endpoints or for missing concentration data. No formal hypothesis testing was conducted. In general, descriptive statistics such as mean, median, standard deviation, interquartile range, and minimum and maximum were used to summarize data for continuous variables, and counts and percentages were used to summarize data for categorical variables.

### Results

### Patient Disposition and Disease Characteristics

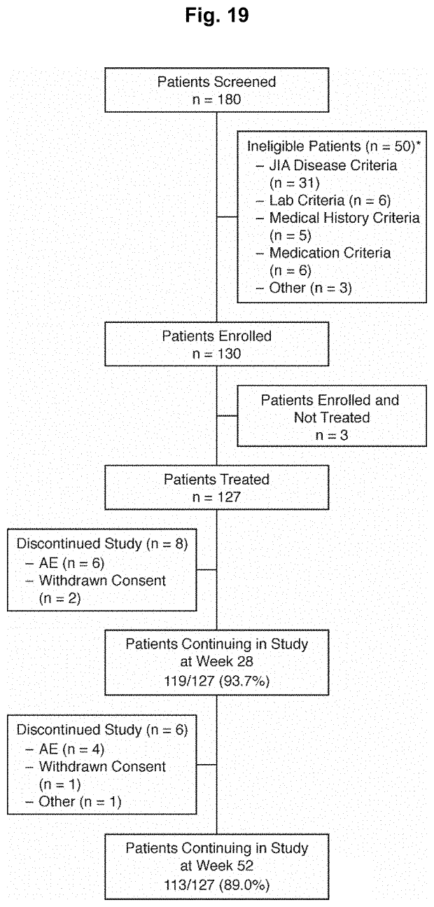

Of the 180 patients screened, 127 (71%) were enrolled and received at least 1 dose of IV golimumab and were included in the full analysis data set (FIG. 19). Of the 127 treated patients, 113 (89%) remained in the study through Week 52. AEs were the primary reason for study discontinuation.

Baseline demographics, disease characteristics, and prior treatment for poly-JIA are summarized in Table 9. Median age at baseline was 13 years, the majority of patients were female (73%) and white (67%), and median weight was 42.4 kg. The majority of patients were classified as RF-negative (43%) and RF-positive (35%) poly-JIA. The most common prior medications were NSAIDs (94%) and systemic glucocorticoids (57%). Of the 25 patients who had received prior anti-TNF therapy, most (80%) had received etanercept. At baseline, 72% of patients were taking NSAIDs, 37% were taking oral glucocorticoids, and 10% were taking an s-DMARD other than MTX. At baseline, 121 (99%) of patients had high disease activity as measured by JADAS 71 (Table 10).

TABLE 9
Baseline demographics, disease characteristics,
and prior arthritis treatment

| Characteristic | Golimumab (N = 127) | |
|---|---|---|
| Age, years | 13.0 | (8.0, 15.0) |
| Female, n (%) | 93 | (73.2) |
| Race, n (%) | | |
| White | 85 | (66.9) |
| Other | 28 | (22.0) |
| Hispanic or Latino, n (%) | 63 | (49.6) |
| Weight, kg | 42.4 | (29.2, 57.0) |
| BSA, m² | 1.3 | (1.0, 1.6) |
| Duration of disease, years | 1.4 | (0.5, 4.0) |
| ILAR classification, n (%) | | |
| Polyarticular rheumatoid factor-negative | 54 | (42.5) |
| Polyarticular rheumatoid factor-positive | 44 | (34.6) |
| Enthesitis-related arthritis | 12 | (9.4) |
| Oligoarticular extended | 8 | (6.3) |
| Juvenile psoriatic arthritis | 5 | (3.9) |
| Systemic with no systemic symptoms but with | 4 | (3.1) |
| polyarticular course | | |
| ANA positive, n (%) | 64 | (50.4) |
| Prior joint procedure or injection, n (%) | 26 | (20.5) |
| Steroid joint injection | 25 | (96.2) |
| Other^{a} | 10 | (38.5) |
| Baseline JIA medications^{b} | | |
| Methotrexate, n (%) | 127 | (100) |
| Methotrexate dose, mg/m²/wk | 13.6 | (4.5) |
| s-DMARDs other than methotrexate, n (%) | 13 | (10.2) |
| Oral glucocorticoids, n (%) | 47 | (37.0) |
| Prednisone or equivalent dose, mg/kg/day | 0.16 | (0.1) |
| NSAIDs, n (%) | 92 | (72.4) |
| Prior JIA medications,^{c} n (%) | | |
| Methotrexate | 127 | (100) |
| s-DMARDs other than methotrexate^{d} | 25 | (19.7) |
| Anti-TNF therapy | 25 | (19.7) |
| b-DMARDs other than anti-TNF therapy | 3 | (2.4) |
| Systemic glucocorticoids | 72 | (56.7) |
| NSAIDs | 119 | (93.7) |

Values are median (IQ range) unless otherwise noted.
^{a}Arthrocentesis, arthroscopy (surgical or diagnostic), osteotomy, tendon surgery
^{b}Baseline JIA medication is any medication used both prior to and after the first study agent administration
^{c}Prior JIA medication is any medication with a start date before the day of the first study agent administration
^{d}Included immunosuppressive agents cyclosporine (n = 2) and azathioprine (n = 1)
ANA, antinuclear antibody;
b-DMARD, biologic disease-modifying anti-rheumatic drug;
BSA, body surface area;
DMARD, disease-modifying antirheumatic drug;
ILAR, International League of Associations for Rheumatology;
IQ, interquartile;
JIA, juvenile idiopathic arthritis;
N, all treated patients;
n, number of patients;
NSAID, nonsteroidal anti-inflammatory drug;
s-DMARD, synthetic disease-modifying antirheumatic drug;
TNF, tumor necrosis factor

TABLE 10
Summary of JIA core set measures and other disease activity parameters (N = 127)

| Characteristic | Baseline | | Week 4 | | Week 28 | | Week 52 | |
|---|---|---|---|---|---|---|---|---|
| JIA core set measures | | | | | | | | |
| MD global of disease | 5.5 | (4.5, 6.8)^{a} | 2.2 | (1.0, 3.8) | 0.5 | (0.1, 1.2) | 0.3 | (0.0, 1.4) |
| activity, 0-10 cm VAS | | | | | | | | |
| Parent assessment | 5.4 | (3.3, 6.9) | 2.6 | (1.1, 5.0) | 1.7 | (0.3, 4.8) | 1.1 | (0.2, 4.2) |
| of overall well- | | | | | | | | |
| being, 0-10 cm VAS | | | | | | | | |
| Number of active joints | 14.0 | (9.0, 22.0) | 6.0 | (2.0, 11.0) | 1.0 | (0.0, 4.0) | 0.0 | (0.0, 3.0) |
| Number of joints with | 10.0 | (4.0, 18.0) | 3.0 | (0.0, 9.0) | 1.0 | (0.0, 4.0) | 1.0 | (0.0, 5.0) |
| limited range of motion | | | | | | | | |
| CHAQ, 0-3 score | 1.25 | (0.8, 1.9) | 0.9 | (0.4, 1.4) | 0.4 | (0.0, 1.1) | 0.4 | (0.0, 1.1) |
| CRP, mg/dL^{b} | 0.5 | (0.1, 1.1) | 0.1 | (0.0, 0.3) | 0.1 | (0.0, 0.7) | 0.1 | (0.0, 0.6) |
| Duration of morning | 40 | (20, 60) | 5 | (0, 30) | 0 | (0, 15) | 0 | (0, 15) |
| stiffness, minutes | | | | | | | | |
| JADAS 71, mean | 28.4 | (26.1, 30.7) | 14.6 | (12.4, 16.8) | 6.8 | (5.2, 8.3) | 5.4 | (3.9, 6.9) |
| (95% confidence interval) | | | | | | | | |
| JADAS 71 high disease | 121 | (99.2) | 73 | (58.9) | 23 | (20.2) | 16 | (14.5) |
| activity >10.5, n (%) | | | | | | | | |
| JADAS 71 moderate disease | 1 | (0.8) | 32 | (25.8) | 37 | (32.5) | 32 | (29.1) |
| activity 3.9-10.5, n (%) | | | | | | | | |

| | | | | | | | |
|---|---|---|---|---|---|---|---|
| JADAS 71 low disease | 0 | 15 | (12.1) | 27 | (23.7) | 23 | (20.9) |
| activity 1.1-3.8, n (%) | | | | | | | |
| JADAS 71 inactive | 0 | 4 | (3.2) | 27 | (23.7) | 39 | (35.5) |
| disease ≤1, n (%) | | | | | | | |

All values are median (IQ range) unless otherwise noted.
^{a}n = 122
^{b}Normal is ≤0.287 mg/dL
95% confidence interval is based on normal approximation: mean ± 1.96 × SD/√N
CHAQ, Childhood Health Assessment Questionnaire;
CRP, C-reactive protein;
IQ, interquartile;
JADAS 71, Juvenile Arthritis Disease Activity Score 71 joints evaluated;
JIA, juvenile idiopathic arthritis;
MD, medical doctor;
VAS, visual analogue scale

### Pharmacokinetics and Immunogenicity

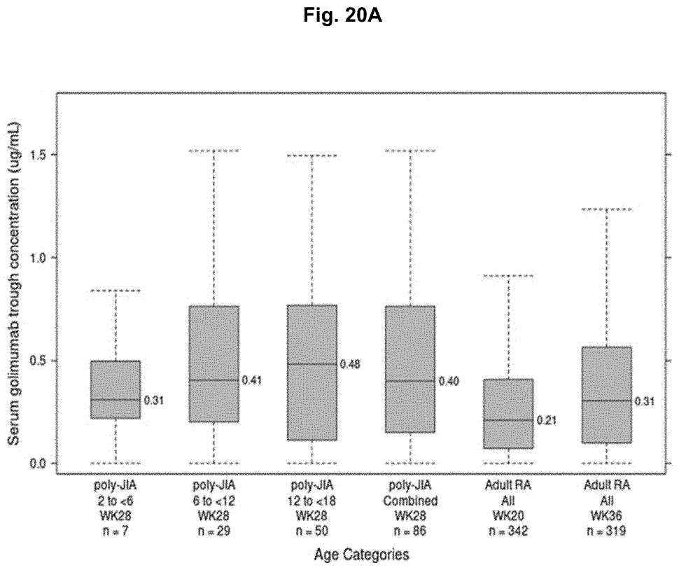

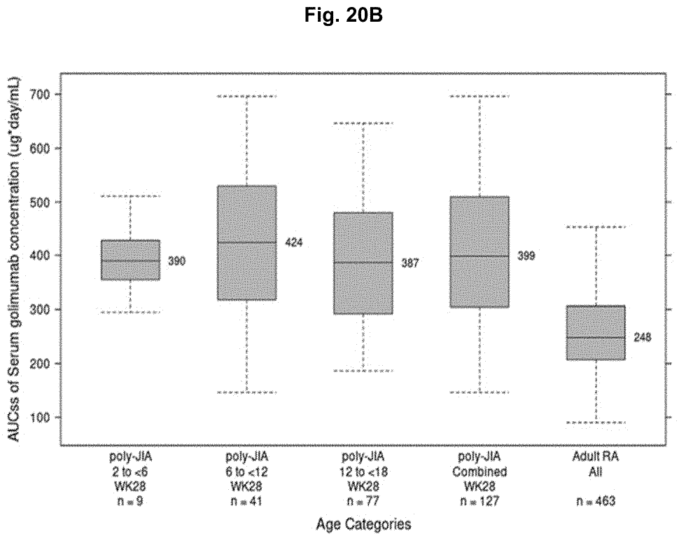

Overall, PK exposure in poly-JIA patients after administration of IV golimumab 80 mg/m² at Week 0, 4, and q8w thereafter was similar to that in the adult RA reference population (FIG. 20A and FIG. 20B). The overall median steady-state trough golimumab concentration in poly-JIA patients was 0.40 μg/mL (mean±SD: 0.50±0.43 μg/mL) at Week 28 and 0.45 μg/mL (mean±SD: 0.52±0.48 μg/mL) at Week 52. Overall median steady-state trough golimumab concentrations were similar across pediatric age categories at Week 28 and similar to the median trough golimumab concentrations observed at Week 36 (0.31 μg/mL; mean±SD 0.41±0.52 μg/mL) in the adult RA reference population (FIG. 20A). The observed median trough golimumab concentrations were also similar across body weight quartiles at Week 28.

The population PK model-predicted median overall AUC for patients with poly-JIA over an 8-week dosing interval was 399 μg·day/mL at Week 28 and 421 μg·day/mL at Week 52. These values were consistent across pediatric age categories (FIG. 20B). The model-predicted AUC values in poly-JIA patients were slightly higher than the AUC (248 μg·day/mL) observed in the adult RA reference population (FIG. 20B); however, monoclonal antibodies have been shown to have moderate to high variability in PK.

Through Week 52, 39 of 125 (31%) treated patients who had appropriate samples were positive for antibodies to golimumab and 24 (19%) were positive for neutralizing antibodies. Peak titer for antibodies to golimumab was <10 in 5 patients, ≥10 to <100 in 17 patients, ≥100 to <1000 in 13 patients, and ≥1000 in 4 patients. Median trough golimumab concentration tended to be lower in antibody-positive patients than in antibody-negative patients (0.00 [n=32] versus 0.61 μg/mL [n=63] at Week 52).

### Effectiveness

As shown in Table 10, improvement from baseline in the JIA ACR component scores was observed as early as Week 4 and maintained from Week 28 through Week 52. At Weeks 28 and 52, respectively, median percent improvement was 92% and 96% for MD global of disease activity, 63% and 70% for parent assessment of overall well-being, 94% and 100% for number of active joints, 89% and 85% for number of joints with limited range of motion, 57% and 63% for physical function by CHAQ, and 53% and 48% for CRP.

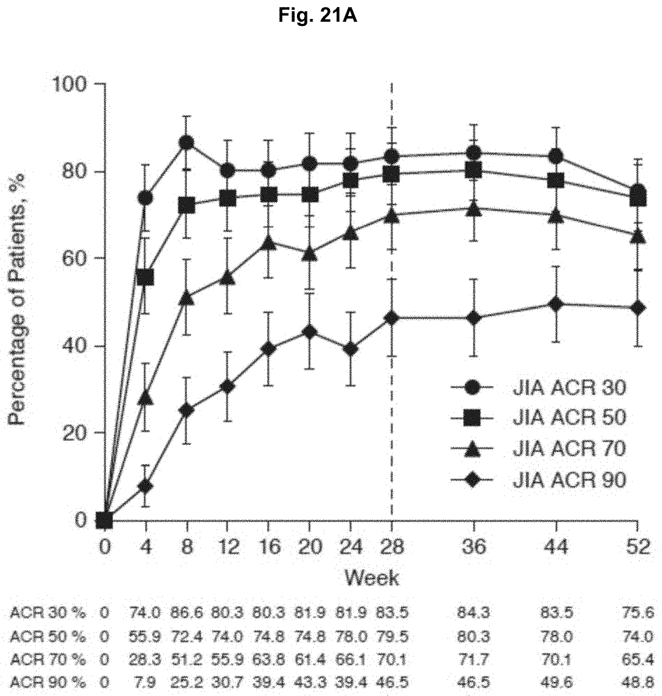

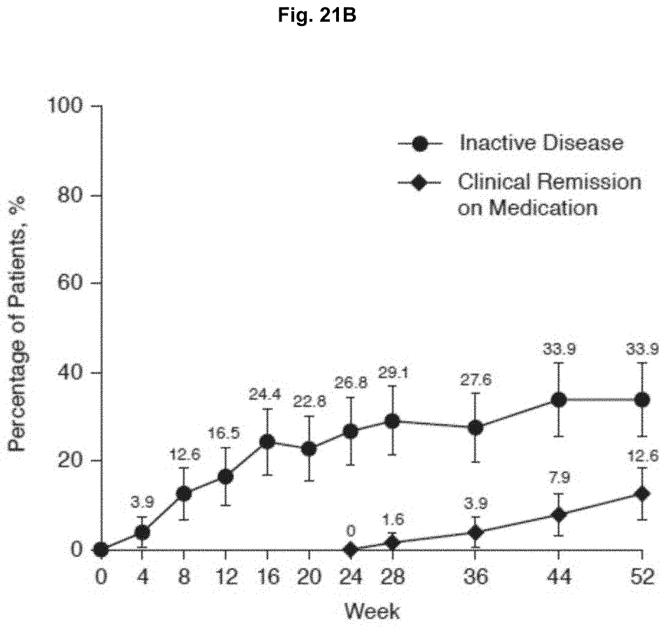

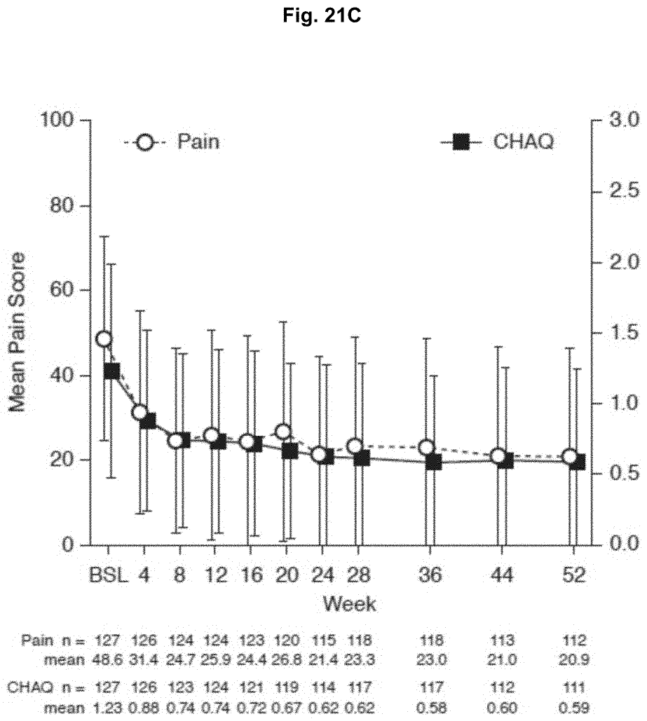

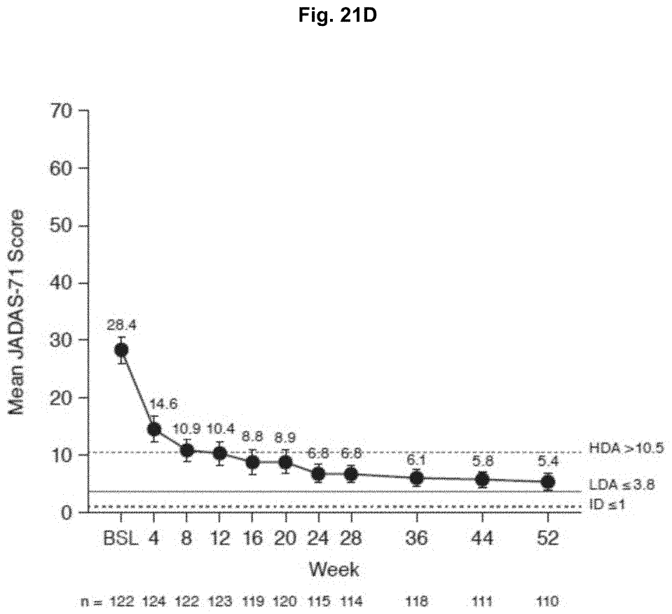

Similarly, JIA ACR 30, 50, 70, and 90 responses were observed as early as Week 4, with more than 50% of patients achieving at least JIA ACR 50 (FIG. 21A). At Week 28, 70% of patients achieved at least a JIA ACR 70 response and nearly half (47%) achieved a JIA ACR 90 response. These response rates were maintained through Week 52. Through Weeks 28 and 52, consistently high JIA ACR response rates were observed across serum trough golimumab concentration quartiles for JIA ACR 30, 50, 70, and 90 (data not shown). At Week 52, the median serum trough golimumab concentration was higher in JIA ACR 30 responders (0.47 μg/mL, n=83) than in non-responders (0.04 μg/mL, n=12); 6 out of the 12 nonresponders were positive for golimumab antibodies.

JIA ACR 30, 50, 70, and 90 response rates among the different poly-JIA subtypes were generally similar to those observed in the overall population; however, response rates were generally lower in patients with systemic poly-JIA with no systemic symptoms but with polyarticular course (at Week 52, 25% had achieved at least a JIA ACR 70 response) and were generally higher in patients with oligoarticular extended or juvenile psoriatic arthritis (at Week 52, 88% and 80%, respectively, had achieved at least a JIA ACR 70 response). It should be noted that these subtypes also had fewer patients than the other subtypes (Table 9). The JIA ACR 30 response rate at Week 52 was also similar among patients with and without prior anti-TNF therapy (76% in both groups).

JIA ACR inactive disease was achieved by 4% of patients as early as Week 4 and increased to 29% at Week 28 and to 34% at Week 52 (FIG. 21B). Clinical remission while on medication was achieved as early as Week 28 by 2% of patients and by 13% of patients at Week 52 (FIG. 21B). Mean improvement from baseline in CHAQ score was observed as early as Week 4 (0.34), increased to 0.62 at Week 28, and remained stable through Week 52 (FIG. 21C). The pattern of improvement was similar for parent assessment of patient pain (FIG. 21C).

A decrease in mean JADAS 71 score was observed as early as Week 4 and continued through Week 52 (FIG. 21D). Mean decrease from baseline in JADAS 71 was −21.28 at Week 28 and −22.18 at Week 52. As early as Week 4, LDA was achieved by 12% of patients and ID was achieved by 3% of patients (Table 10). By Week 52, 21% of patients achieved LDA and 36% achieved ID.

### Safety

Through Week 52, most patients (85%) experienced at least 1 AE; 7% experienced at least 1 serious AE (SAE) and 9% experienced at least 1 AE that lead to discontinuation (Table 11). More than half of treated patients (65%) experienced 1 or more infections, 6% of patients experienced 1 or more serious infections, and 1 patient experienced an opportunistic infection. The proportion of patients with infusion reactions was low, none of the reactions was severe or led to treatment discontinuation, and there was no impact of antibodies to golimumab on infusion reactions. No active tuberculosis, demyelinating event, or anaphylactic or serum sickness reactions were reported. Systemic lupus erythematosus was reported in 1 patient; the event was considered to be nonserious and not related to golimumab. There were no deaths reported through Week 52, but 1 death due to septic shock (likely due to constipation leading to bacterial translocation through the gut wall) was reported after Week 52. The patient received their last dose of IV golimumab at Week 76 and died at Week 78. The event was considered serious, severe in intensity, and probably related to golimumab.

AEs were generally consistent with the established safety profiles for golimumab and other anti-TNF therapies. The MedDRA system organ class (SOC) with the highest incidence of AEs at Week 52 was Infections and infestations (67%) (Table 11). The most commonly reported AEs were upper respiratory tract infection (21.3%) and nasopharyngitis (18%). Nine (7%) patients experienced SAEs through Week 52; 1 (1%) patient each experienced herpes zoster disseminated, infective exacerbation of bronchiectasis, sepsis, Varicella, mycosis fungoides, suicidal ideation, cellulitis, pneumonia, pneumonia streptococcal, and pleural effusion (pneumonia streptococcal and plural effusion were reported in the same patient). All of these events, except varicella, cellulitis, and pneumonia, resulted in permanent discontinuation of IV golimumab. New-onset, anterior uveitis in both eyes was reported in 1 patient through Week 52. The incident was considered incipient/very mild and did not require treatment.

Of 115 patients evaluated at Week 24, 57 were ANA negative at baseline and 13 (23%) were newly ANA positive at Week 24. Of 110 patients evaluated at Week 52, 51 were ANA negative at baseline and 13 (26%) of those patients were newly ANA positive at Week 52. Of these newly positive patients, 7 had been ANA negative at Week 24 and 6 had been ANA positive. Six of the patients who were ANA positive at Week 24 became ANA negative at Week 52 and 1 had discontinued the study. At Week 24, titers were 1:40 in 11 patients and 1:160 in 2 patients. At Week 52, titers were 1:40 in 8 patients, 1:80 in 3 patients, and 1:160 in 2 patients. The assay was kept stable throughout the study. None of the newly positive patients at Week 24 and 52 had a history of ANA positivity and none were positive for anti-dsDNA antibodies at baseline, Week 28, or Week 52.

TABLE 11
Summary of adverse events through Week 52

| | Golimumab (N = 127) | |
|---|---|---|
| Average duration of follow-up, weeks | 49.8 | |
| Average exposure, number of administrations | 6.6 | |
| Patients who discontinued study agent due | 11 | (8.7) |
| to ≥1 AE | | |
| Patients with ≥1 AE | 108 | (85.0) |
| Patients with ≥1 severe AE | 5 | (3.9) |
| Patients with ≥1 SAE | 9 | (7.1) |
| AEs per 100 patient-years exposure, | 359.6 | (326.7, 394.9) |
| n (95% CI) | | |
| SAEs per 100 patient-years exposure, | 8.2 | (4.0, 15.1) |
| n (95% CI) | | |
| Deaths^{a} | 0 | |
| Patients with ≥1 infection | 83 | (65.4) |
| ≥1 serious infection | 7 | (5.5) |
| ≥1 opportunistic infection | 1 | (0.8) |
| Infections per 100 patient-years exposure, | 151.4 | (130.3, 174.9) |
| n (95% CI) | | |
| Serious infection per 100 patient-years | 6.6 | (2.8, 13.0) |
| exposure, n (95% CI) | | |
| Patients with ≥1 infusion-related reaction | 3 | (2.4) |
| Patients with ≥1 malignancy^{b} | 1 | (0.8) |
| Patients with active tuberculosis | 0 | |
| ANA/anti-dsDNA antibodies^{c} | 13 | (25.5) |
| Common AEs (occurring in ≥5% of patients) | | |
| by SOC and related PTs | | |
| Infections and infestations | 85 | (66.9) |
| Upper respiratory tract infection | 27 | (21.3) |
| Nasopharyngitis | 23 | (18.1) |
| Gastrointestinal disorders | 30 | (23.6) |
| Nausea | 11 | (8.7) |
| Vomiting | 10 | (7.9) |
| Abdominal pain | 8 | (6.3) |
| Musculoskeletal and connective tissue | 24 | (18.9) |
| disorders | | |
| Juvenile idiopathic arthritis | 14 | (11.0) |
| Nervous system disorders | 20 | (15.7) |
| Headache | 14 | (11.0) |
| Investigations | 13 | (10.2) |
| Alanine aminotransferase increased | 7 | (5.5) |

All values are n (%) unless otherwise noted.
^{a}One death due to septic shock was reported at Week 78
^{b}Mycosis fungoides
^{c}Newly developed; out of 51 patients who were ANA negative at baseline
AE, adverse event;
ANA, antinuclear antibody;
anti-dsDNA, anti-double-stranded deoxyribonucleic acid;
CI, confidence interval;
N, all treated patients;
n, number of patients;
PT, preferred term;
SAE, serious adverse event;
SOC, system organ class

### Discussion

In this open-label Phase 3 study in pediatric patients with poly-JIA, IV golimumab plus MTX provided PK exposure similar to that found to be effective in adults with RA. Median trough serum golimumab concentrations and AUC_{SS} were generally maintained over time and were similar across age groups and body weight quartiles, indicating that BSA-based dosing was appropriate to achieve similar PK exposure across the entire poly-JIA age and body-weight range.

It has been well recognized that cross-study comparisons of steady-state trough levels can be challenging, particularly when the trough levels are relatively low and, thus, highly variable from study to study. To put the interstudy variability into the context of cross-study comparisons, the steady-state trough concentrations observed in poly-JIA were also compared with all 3 adult Phase 3 studies with IV golimumab, including RA, psoriatic arthritis, and ankylosing spondylitis. The median (mean±SD) steady-state trough serum golimumab concentration in poly-JIA patients at Week 28 0.40 μg/mL (0.50±0.43 μg/mL) was within the range of those observed for adults with RA, psoriatic arthritis, or ankylosing spondylitis at Week 36 receiving IV administration of golimumab (0.31 [0.41±0.52], 0.61 [0.69±0.58], and 0.71 [0.74±0.51 ]μg/mL, respectively). Taking interstudy variability into consideration, these PK data support the conclusion that the steady-state golimumab concentrations observed in children in this study were generally similar to those observed in the adult RA reference population.

Notably, patients in the highest weight quartile group in this pediatric study had a mean body weight of 73 kg (range: 57.00 to 142.70 kg), which was similar to the mean body weight of the adult RA reference population (72 kg; range: 39.00 to 125.00 kg). In addition, the calculated total dose difference for the 2 mg/kg dose used in the adult RA study versus the 80 mg/m² dose used in this pediatric study yielded a small dose difference (mean 2%; range: −13% to 16%) for the highest body weight quartile group, demonstrating that the poly-JIA patients in this group received golimumab doses comparable to those in the adult RA reference population. Therefore, the PK exposure from the highest body weight quartile group provides an internal reference for PK comparison across different age and weight subgroups to demonstrate that PK exposure in all the poly-JIA subgroups was similar to that in the adult RA reference population.

IV golimumab led to a reduction in clinical signs and symptoms of poly-JIA that was generally maintained through Week 52. Consistently high JIA ACR 30, 50, 70, and 90 response rates were observed overall, across the trough serum golimumab concentration quartiles for JIA ACR response and poly-JIA subtypes, and in patients with and without prior anti-TNF exposure. The JIA ACR response rates and the other clinical responses we observed with IV golimumab in this study are consistent with those reported for SC golimumab and other b-DMARDs in similar Phase 3 poly-JIA studies.

Median trough golimumab concentration was lower in ADA-positive patients compared with ADA-negative patients and in JIA ACR 30 nonresponders compared with responders. The low median golimumab concentration in JIA ACR 30 non-responders overall was because 6 of 12 JIA ACR 30 non-responders were ADA positive and had median golimumab concentrations below the lower limit of quantitation. However, it does not appear that ADA status had an effect on the efficacy profile because 6 of the 12 non-responders at Week 52 were also ADA negative. In addition, JIA ACR 30 response rates were similar in ADA-positive and ADA-negative patients (79% versus 74%, respectively).

The overall safety profile of IV golimumab in patients with poly-JIA through Week 52 was consistent with that of IV golimumab in adult patients with rheumatic disease and SC golimumab in patients with poly-JIA. Although there were no deaths through Week 52, 1 death, which was considered to be probably related to IV golimumab, was reported at Week 78. No deaths have been reported with SC golimumab and other b-DMARDs in similar Phase 3 poly-JIA studies. Thirteen patients had newly developed ANA antibodies at Week 52 and none of those patients had anti-dsDNA antibodies.

It is an ethical requirement of the PRINTO and PRCSG networks that companies involved in trials for registration purposes should continue to provide the drug to children enrolled in a clinical trial until an alternative method of drug provision is identified. As previously reported, this requirement is of particular importance for countries with less resources where children might not have public or private insurance to cover the high cost of b-DMARDs. For this trial, drug provision was stopped after 252 weeks for the children enrolled in the trial who have reached the age of 18 years. IV golimumab is currently marketed for RA, PsA, or AS in many of the countries (7 out of 9) participating in the trial.

A limitation of this study is its open-label, nonrandomized, and uncontrolled design. The study was designed this way with the intent to extrapolate the results from efficacy trials in adults.

In conclusion, IV golimumab 80 mg/m² at Weeks 0 and 4 and then q8w through Week 52 with weekly MTX provided adequate PK exposure for clinical efficacy in patients with active poly-JIA, including a subset of patients with prior exposure to anti-TNF therapy. This IV golimumab regimen was also well tolerated in this patient population.

What is claimed is:

1. A method of treating juvenile idiopathic arthritis (JIA) in pediatric patients, the method comprising administering an intravenous (IV) dose of an anti-TNF antibody to the pediatric patient, wherein the anti-TNF antibody comprises a heavy chain (HC) comprising an amino acid sequence of SEQ ID NO:36 and a light chain (LC) comprising an amino acid sequence of SEQ ID NO:37, and wherein
a. >50% of the pediatric patients meet the criteria for JIA American College of Rheumatology (JIA ACR) 30, JIA ACR 50, and JIA ACR 70 after 52 weeks of treatment;
b. >20% of the pediatric patients have a Juvenile Arthritis Disease Activity Score counting 71 joints (JADAS 71) for low disease activity after 52 weeks of treatment;
c. the pediatric patients meet the criteria for JIA American College of Rheumatology (JIA ACR) inactive disease after 52 weeks of treatment; or
d. the pediatric patients meet the criteria for JIA American College of Rheumatology (JIA ACR) clinical remission after 52 weeks of treatment.

2. The method of claim 1, wherein said pediatric patients are 2 to <18 years old.

3. The method of claim 1, wherein said juvenile idiopathic arthritis (JIA) is polyarticular juvenile idiopathic arthritis (pJIA).

4. The method of claim 1, wherein the IV dose is 80 mg/m² of the anti-TNF antibody, at weeks 0, 4, and then every 8 weeks thereafter.

5. The method of claim 1, wherein the method further comprises administering methotrexate (MTX) to the pediatric patients.

6. The method of claim 1, wherein >30% of the pediatric patients meet the criteria for JIA ACR inactive disease after 52 weeks of treatment.

7. The method of claim 1, wherein >10% of the pediatric patients meet the criteria for JIA ACR clinical remission after 52 weeks of treatment.


SEQUENCE LISTING (45 sequences)

SEQ ID NO: 1 - protein, 5 aa, Homo sapiens
  MISC_FEATURE (1)..(5): Heavy Chain complementarity determining region 1 (CDR1).
SYAMH

SEQ ID NO: 2 - protein, 17 aa, Homo sapiens
  MISC_FEATURE (1)..(17): Heavy Chain complementarity determining region 2 (CDR2).
  MISC_FEATURE (1)..(1): Xaa at position 1 is selected from Ile, Phe or Val.
  MISC_FEATURE (2)..(2): Xaa at position 2 is selected from Ile or Met.
  MISC_FEATURE (3)..(3): Xaa at position 3 is selected from Ser or Leu.
  MISC_FEATURE (4)..(4): Xaa at position 4 is selected from Tyr or Phe.
  MISC_FEATURE (10)..(10): Xaa at position 10 is selected from Lys or Tyr.
  MISC_FEATURE (11)..(11): Xaa at position 11 is selected from Ser or Tyr.
  MISC_FEATURE (17)..(17): Xaa at position 17 is selected from Asp or Gly.
XXXXDGSNKXXADSVKX

SEQ ID NO: 3 - protein, 17 aa, Homo sapiens
  MISC_FEATURE (1)..(17): Heavy Chain complementarity determining region 3 (CDR3).
  MISC_FEATURE (4)..(4): Xaa at position 4 is selected from Ile or Val.
  MISC_FEATURE (5)..(5): Xaa at position 5 is selected from Ser, Ala or Gly.
  MISC_FEATURE (9)..(9): Xaa at position 9 is selected from Asn or Tyr.
DRGXXAGGXYYYYGMDV

SEQ ID NO: 4 - protein, 11 aa, Homo sapiens
  MISC_FEATURE (1)..(11): Light Chain complementarity determining region 1 (CDR1).
  MISC_FEATURE (7)..(7): Xaa at position 7 is selected from Ser or Tyr.
RASQSVXSYLA

SEQ ID NO: 5 - protein, 7 aa, Homo sapiens
  MISC_FEATURE (1)..(7): Light Chain complementarity determining region 2 (CDR2).
DASNRAT

SEQ ID NO: 6 - protein, 10 aa, Homo sapiens
  MISC_FEATURE (1)..(10): Light Chain complementarity determining region 3 (CDR3).
QQRSNWPPFT

SEQ ID NO: 7 - protein, 126 aa, Homo sapiens
  MISC_FEATURE (1)..(126): heavy chain variable region sequences as presented in original Figure 4
  MISC_FEATURE (1)..(30): framework 1
  MISC_FEATURE (28)..(28): Xaa at position 28 is selected from Ile or Thr.
  MISC_FEATURE (31)..(35): complementarity determining region 1 (CDR1).
  MISC_FEATURE (36)..(49): framework 2
  MISC_FEATURE (43)..(43): Xaa at position 43 is selected from Lys or Asn.
  MISC_FEATURE (50)..(66): complementarity determining region 2 (CDR2).
  MISC_FEATURE (50)..(50): Xaa at position 50 is selected from Ile, Phe or Val.
  MISC_FEATURE (51)..(51): Xaa at position 51 is selected from Ile or Met.
  MISC_FEATURE (52)..(52): Xaa at position 52 is selected from Ser or Leu.
  MISC_FEATURE (53)..(53): Xaa at position 53 is selected from Tyr or Phe.
  MISC_FEATURE (59)..(59): Xaa at position 59 is selected from Lys or Tyr.
  MISC_FEATURE (60)..(60): Xaa at position 60 is selected from Ser or Tyr.
  MISC_FEATURE (66)..(66): Xaa at position 66 is selected from Asp or Gly.
  MISC_FEATURE (67)..(98): framework 3
  MISC_FEATURE (70)..(70): Xaa at position 70 is selected from Val or Ile.
  MISC_FEATURE (75)..(75): Xaa at position 75 is selected from Ser or Pro.
  MISC_FEATURE (78)..(78): Xaa at position 78 is selected from Thr or Ala.
  MISC_FEATURE (80)..(80): Xaa at position 80 is selected from Tyr or Phe.
  MISC_FEATURE (94)..(94): Xaa at position 94 is selected from Tyr or Phe.
  MISC_FEATURE (99)..(115): complementarity determining region 3 (CDR3).
  MISC_FEATURE (102)..(102): Xaa at position 102 is selected from Ile or Val.
  MISC_FEATURE (116)..(126): J6 region
QVQLVESGGGVVQPGRSLRLSCAASGFXFSSYAMHWVRQAPGXGLEWVAXXXXDGSNKXXADSVKXRFTXSRDNXKNXLXLQMNSLRAEDTAVXYCARDRGXAAGGNYYYYGMDVWGQGTTVTVSS

SEQ ID NO: 8 - protein, 108 aa, Homo sapiens
  MISC_FEATURE (1)..(108): light chain variable region sequences as presented in original Figure 5
  MISC_FEATURE (1)..(23): framework 1
  MISC_FEATURE (24)..(34): complementarity determining region 1 (CDR1).
  MISC_FEATURE (35)..(49): framework 2
  MISC_FEATURE (50)..(56): complementarity determining region 2 (CDR2).
  MISC_FEATURE (57)..(88): framework 3
  MISC_FEATURE (89)..(98): complementarity determining region 3 (CDR3).
  MISC_FEATURE (99)..(108): J3 region
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIK

SEQ ID NO: 9 - protein, 157 aa, Homo sapiens [sequence omitted]

SEQ ID NO: 10 - DNA, 18 bp, Homo sapiens
ttggtccagtcggactgg

SEQ ID NO: 11 - DNA, 18 bp, Homo sapiens
cacctgcactcggtgctt

SEQ ID NO: 12 - DNA, 30 bp, Homo sapiens
cactgttttgagtgtgtacgggcttaagtt

SEQ ID NO: 13 - DNA, 18 bp, Homo sapiens
gccgcacgtgtggaaggg

SEQ ID NO: 14 - DNA, 25 bp, Homo sapiens
agtcaaggtcggactggcttaagtt

SEQ ID NO: 15 - DNA, 28 bp, Homo sapiens
gttgtcccctctcacaatcttcgaattt

SEQ ID NO: 16 - DNA, 18 bp, Homo sapiens
ggcggtagactactcgtc

SEQ ID NO: 17 - protein, 7 aa, Homo sapiens
MDWTWSI

SEQ ID NO: 18 - DNA, 35 bp, Homo sapiens
tttcgtacgccaccatggactggacctggagcatc

SEQ ID NO: 19 - DNA, 34 bp, Homo sapiens
tttcgtacgccaccatggggtttgggctgagctg

SEQ ID NO: 20 - DNA, 35 bp, Homo sapiens
tttcgtacgccaccatggagtttgggctgagcatg

SEQ ID NO: 21 - DNA, 35 bp, Homo sapiens
tttcgtacgccaccatgaaacacctgtggttcttc

SEQ ID NO: 22 - DNA, 35 bp, Homo sapiens
tttcgtacgccaccatggggtcaaccgccatcctc

SEQ ID NO: 23 - protein, 6 aa, Homo sapiens
TVTVSS

SEQ ID NO: 24 - DNA, 36 bp, Homo sapiens
gtgccagtggcagaggagtccattcaagcttaagtt

SEQ ID NO: 25 - protein, 5 aa, Homo sapiens
MDMRV

SEQ ID NO: 26 - DNA, 31 bp, Homo sapiens
tttgtcgacaccatggacatgagggtcctcc

SEQ ID NO: 27 - DNA, 28 bp, Homo sapiens
tttgtcgacaccatggaagccccagctc

SEQ ID NO: 28 - protein, 6 aa, Homo sapiens
TKVDIK

SEQ ID NO: 29 - DNA, 41 bp, Homo sapiens
ctggtttcacctatagtttgcattcagaattcggcgccttt

SEQ ID NO: 30 - DNA, 35 bp, Homo sapiens
catctccagagacaattccaagaacacgctgtatc

SEQ ID NO: 31 - DNA, 35 bp, Homo sapiens
gtagaggtctctgttaaggttcttgtgcgacatag

SEQ ID NO: 32 - protein, 19 aa, Homo sapiens
  MISC_FEATURE (1)..(19): Signal sequence for heavy chain variable region sequences as presented in original Figure 4
MGFGLSWVFLVALLRGVQC

SEQ ID NO: 33 - protein, 20 aa, Homo sapiens
  MISC_FEATURE (1)..(20): Signal sequence for light chain variable region sequences as presented in original Figure 5
MEAPAQLLFLLLLWLPDTTG

SEQ ID NO: 34 - DNA, 428 bp, Homo sapiens
atggggtttgggctgagctgggttttcctcgttgctcttttaagaggtgtccagtgtcaggtgcagctggtggagtctgggggaggcgtggtccagcctgggaggtccctgagactctcctgtgcagcctctggttcaccttcagtagctatgctatgcactgggtccgccaggctccggcaaggggctggagtgggtggcagttatatcatatgatggaaaataaatactacgcagactccgtgaagggccgattcaccatctagagacaattccaagaacacgctgtatctgcaaatgaacagccagagctgaggacacggctgtgtattactgtgcgagagatcgaggtatatcagcaggtggaatactactactactacggtatggacgtctgggggcaagggaccacggtcaccgtctcctca

SEQ ID NO: 35 - DNA, 387 bp, Homo sapiens
atggaagccccagctcagcttctcttcctcctgctactctggctcccagataccaccggagaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccattcactttcggccctgggaccaaagtggatatcaaacgt

SEQ ID NO: 36 - protein, 456 aa, Homo sapiens
  MISC_FEATURE (1)..(456): golimumab heavy chain (HC)
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 215 aa, Homo sapiens
  MISC_FEATURE (1)..(215): golimumab light chain (LC)
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 126 aa, Homo sapiens
  MISC_FEATURE (1)..(126): golimumab variable heavy chain region
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 39 - protein, 111 aa, Homo sapiens
  MISC_FEATURE (1)..(111): golimumab variable light chain region
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTV

SEQ ID NO: 40 - protein, 5 aa, Homo sapiens
  MISC_FEATURE (1)..(5): golimumab complementarity determining region heavy chain 1 (CDRH1)
SYAMH

SEQ ID NO: 41 - protein, 17 aa, Homo sapiens
  MISC_FEATURE (1)..(17): golimumab antibody complementarity determining region heavy chain 2 (CDRH2)
FMSYDGSNKKYADSVKG

SEQ ID NO: 42 - protein, 17 aa, Homo sapiens
  MISC_FEATURE (1)..(17): golimumab complementarity determining region heavy chain 3 (CDRH3)
DRGIAAGGNYYYYGMDV

SEQ ID NO: 43 - protein, 11 aa, Homo sapiens
  MISC_FEATURE (1)..(11): golimumab complementarity determining region light chain 1 (CDRL1)
RASQSVYSYLA

SEQ ID NO: 44 - protein, 7 aa, Homo sapiens
  MISC_FEATURE (1)..(7): golimumab complementarity determining region light chain 2 (CDRL2)
DASNRAT

SEQ ID NO: 45 - protein, 10 aa, Homo sapiens
  MISC_FEATURE (1)..(10): golimumab complementarity determining region light chain 3 (CDRL3)
QQRSNWPPFT